(19) United States
(12) Patent Application Publication
(10) Pub. No.: US 2024/0316099 A1
(43) Pub. Date: Sep. 26, 2024

(54) CYTOTOXIC AND COSTIMULATORY CHIMERIC ANTIGEN RECEPTORS

(71) Applicant: Baylor College of Medicine, Houston, TX (US)

(72) Inventors: Robin Parihar, Pearland, TX (US); Stephanie Fetzko, Houston, TX (US); Tim Sauer, Mannheim (DE)

(21) Appl. No.: 18/245,281

(22) PCT Filed: Sep. 27, 2021
(86) PCT No.: PCT/US2021/071608

§ 371 Date: Mar. 14, 2023

Related U.S. Application Data
(60) Provisional application No. 63/085,931, filed on Sep. 30, 2020.

(51) Int. Cl. A61K 35/17; A61K 39/00; C07K 14/705; C07K 16/30; C12N 15/86

(52) U.S. Cl. CPC A61K 35/17; A61K 39/4613; A61K 39/4631; A61K 39/46447; A61K 39/464471; C07K 14/70503; C07K 16/3084; C07K 16/3092; C12N 15/86; A61K 2239/13; A61K 2239/21; C07K 2317/622; C07K 2319/03

(57) ABSTRACT

Disclosed herein are methods and compositions for cell-based immunotherapies that simultaneously target the tumor microenvironment (TME) via NKG2D ligands and tumor cells via tumor-associated antigens, specifically using immune effector cells as the platform due to their reduced toxicity against normal tissue. In some embodiments, immune effector cells co-express an NKG2D cytotoxic CAR and a CAR directed against a tumor-associated antigen that provides costimulatory signals to the immune effector cell, thus killing only in the presence of both antigens specifically within the TME. In contrast, within normal tissue that might express the tumor-associated antigen, but where self-HLA is also expressed, the costimulatory signal by itself is insufficient for immune effector cell activation, thereby preventing off-tumor toxicity.

### CROSS-REFERENCE TO RELATED APPLICATIONS

This application is a national phase application under 35 U.S.C. § 371 that claims priority to International Application No. PCT/US2021/071608 filed Sep. 27, 2021, which claims priority to U.S. Provisional Application No. 63/085,931 filed Sep. 30, 2020, all of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Oct. 12, 2021, is named BAYM_P0295WO-1001182723_SL.txt and is 138,587 bytes in size.

### TECHNICAL FIELD

This disclosure relates at least to the fields of cancer biology, cell biology, immunology, and medicine.

### BACKGROUND

Solid tumors are refractory to cellular immunotherapies in part because they contain suppressive immune effectors such as myeloid-derived suppressor cells (MDSCs) that inhibit cytotoxic lymphocytes.

The tumor microenvironment (TME) comprises cancer-associated fibroblasts, neo-vasculature, and immune infiltrates partially consisting of immunosuppressive cells such as myeloid-derived suppressor cells (MDSCs), inhibitory macrophages (M2s) and regulatory T cells (Tregs) (14, 15). MDSCs exert multiple immunosuppressive effects including inhibition of T-cell cytotoxicity and proliferation, decreased antigen presentation, and secretion of immune toxic metabolites (16). Solid tumors recruit inhibitory cells such as MDSCs (6). These immature myeloid cells are a component of innate immunity and strengthen the suppressive tumor microenvironment (TME; 7, 8). The frequency of circulating or intratumoral MDSCs correlates with cancer stage, disease progression, and resistance to standard chemotherapy and radiotherapy (9). MDSCs also correlate with poor prognosis and decreased effectiveness of immunotherapy (17). Given their impact on promoting an inhibitory TME with clinical consequences, MDSCs represent an important target for weakening the solid tumor TME.

Immunotherapy with T lymphocytes engineered to express chimeric antigen receptors (CARs) that target antigen-expressing tumors have shown promise in preclinical models. T lymphocytes can be engineered to target tumor-associated antigens by forced expression of chimeric antigen receptors (CAR; 1). Although successful when directed against leukemia-associated antigens such as CD19 (2, 3), CAR-T cell therapy for solid tumors has been less effective, with best responses in patients with minimal disease (4, 5).

Thus, key obstacles exist to designing efficacious cell therapies for solid tumors, including combating the suppressive TME while also limiting off-tumor toxicity. One such challenge in designing immunotherapies for solid tumors is identification of ideal tumor-associated antigens (TAAs) as targets. Few tumor-specific antigens exist for sarcomas, and therefore, identification of tumor targets that ideally maximize tumor killing while minimizing toxicity has been challenging. Thus, there is a critical need to develop approaches that safely target both the TME and tumor-associated antigens in solid tumors.

The present disclosure provides a solution to the need for a cell therapy for solid tumors. More specifically, disclosed herein are strategies to reverse the suppressive tumor microenvironment (TME) and also attract and activate immune effectors with antitumor activity by using genetically enhanced natural killer (NK) cells to counter the suppressive TME of solid tumors and provide tumor-specific killing in a manner that limits off-tumor toxicity.

### BRIEF SUMMARY

Given their potent antigen non-restricted cytotoxicity and extensive immune-stimulatory properties, NK cells are an attractive platform for targeted cellular immunotherapy, and embodiments of the disclosure concern compositions and methods which utilize NK cells in treating cancer. Melanoma cellular adhesion molecule (MCAM), also known as MUC18 or CD146, is a highly differentially-expressed molecule identified in several solid tumors, and it has been studied extensively in malignant melanoma and its expression correlates with cancer growth and metastases (13). Additionally, several studies have reported NKG2D ligand expression on solid tumors including pediatric sarcomas (19, 20).

Accordingly, provided herein, in some aspects, are compositions and methods of using the compositions which comprise CARs to target antigen-expressing tumors, and in some embodiments, a first chimeric antigen receptor polypeptide which comprises one or more antibodies or fragments thereof that binds to one or more cancer-associated antigens such as MUC18 or a binding region thereof is provided. Also provided herein, in some aspects, are compositions and methods of using the compositions which comprise NKG2D CARs (including to target the TME), and in some embodiments, a second chimeric antigen receptor polypeptide which comprises an NKG2D receptor or fragment thereof which binds NKG2D is provided. Further provided herein, in some aspects, are compositions and methods of using the compositions which comprise a single CAR to target antigen-expressing tumors, and in some embodiments, a chimeric antigen receptor polypeptide which comprises one or more antibodies or fragments thereof that binds to one or more cancer-associated antigens such as MUC18 or a binding region thereof is provided.

Particular embodiments of the disclosure utilize NK cells genetically engineered to express the first CAR and the second CAR such that the NK expresses both an anti-tumor antigen antibody, for example, a MUC18 antibody to recognize MUC18 expressed in the TME of solid tumors, and a chimeric NKG2D receptor which recognizes NKG2D ligand+ tumors or cells in the TME and MDSCs of the TME. In some embodiments, recognition of MUC18 and expression of a chimeric NKG2D receptor increases the likelihood of NK activation at tumor sites, thereby increasing specificity and safety of a composition for treating cancer.

Disclosed herein, in some aspects, is a composition comprising one or two chimeric antigen receptor (CAR) polypeptides, the first or only CAR polypeptide comprising one or more antibodies or fragments thereof that binds to one or more cancer-associated antigens or a binding region thereof, and the second CAR polypeptide, when present, comprising an NKG2D receptor or fragment thereof.

Disclosed herein, in some aspects, is a composition comprising an immune effector cell expressing one or two chimeric antigen receptor (CAR) polypeptides, the first or only CAR polypeptide comprising one or more antibodies or fragments thereof that binds to one or more cancer-associated antigens or a binding region thereof, and the second CAR polypeptide, when present, comprising an NKG2D receptor or fragment thereof.

Disclosed herein, in some aspects, is a method for stimulating an immune cell-mediated immune response to a target cell population and/or tissue in a mammal, wherein the target cell population and/or tissue express MUC18 and/or one or more NKG2D ligands, the method comprising administering to a mammal in need thereof a therapeutically effective amount of a composition comprising immune effector cells expressing one or two chimeric antigen receptor (CAR) polypeptides, the first or only CAR polypeptide comprising one or more antibodies or fragments thereof that binds to one or more cancer-associated antigens or a binding region thereof, and the second CAR polypeptide, when present, comprising an NKG2D receptor or fragment thereof.

Disclosed herein, in some aspects, is a method of generating a persisting population of genetically engineered immune cells in a mammal, said method comprising administering to the mammal a therapeutically effective amount of a composition comprising immune effector cells genetically modified to express one or two chimeric antigen receptor (CAR) polypeptides, the first or only CAR polypeptide comprising one or more antibodies or fragments thereof that binds to one or more cancer-associated antigens or a binding region thereof, and the second CAR polypeptide, when present, comprising an NKG2D receptor or fragment thereof, wherein the persisting population of genetically engineered immune cells persists in the mammal for at least about one month up to at least about one year after administration.

Disclosed herein, in some aspects, is a method of expanding a population of genetically engineered immune cells in a mammal, the method comprising administering to the mammal a therapeutically effective amount of a composition comprising immune effector cells genetically modified to express one or two chimeric antigen receptor (CAR) polypeptides, the first or only CAR polypeptide comprising one or more antibodies or fragments thereof that binds to one or more cancer-associated antigens or a binding region thereof, and the second CAR polypeptide, when present, comprising an NKG2D receptor or fragment thereof, wherein the administered genetically engineered immune cell produces a population of progeny immune cells in the mammal.

Disclosed herein, in some aspects, is a method of treating a mammal with a cancer comprising cells that express MUC18 and/or one or more NKG2D ligands, the method comprising administering to a mammal in need thereof a therapeutically effective amount of a composition comprising immune effector cells genetically modified to express one or two chimeric antigen receptor (CAR) polypeptides, the first or only CAR polypeptide comprising one or more antibodies or fragments thereof that binds to one or more cancer-associated antigens or a binding region thereof, and the second CAR polypeptide, when present, comprising an NKG2D receptor or fragment thereof.

Disclosed herein, in some aspects, is a method for treatment of cancer comprising the steps of contacting an immune effector cell with a cancer cell or MDSC, M2-TAM, or Treg within the cancer microenvironment of a mammal and inducing apoptosis of the cancer cell, wherein the immune effector cell is genetically modified to express one or two chimeric antigen receptor (CAR) polypeptides, the first or only CAR polypeptide comprising one or more antibodies or fragments thereof that binds to one or more cancer-associated antigens or a binding region thereof, and the second CAR polypeptide, when present, comprising an NKG2D receptor or fragment thereof.

In some embodiments, the first or only CAR polypeptide comprises: the one or more antibodies or fragments thereof; a transmembrane domain; at least one costimulatory signaling region; and optionally, a detection molecule.

In some embodiments, the one or more cancer-associated antigens or binding regions thereof bound by the one or more antibodies or fragments thereof comprise CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, MUC18, NKG2D, NY-ESO-1, PSCA, WT-1, or any combination thereof. In some embodiments, the one or more cancer-associated antigens or binding regions thereof bound by the one or more antibodies or fragments thereof is MUC18. In some embodiments, the one or more antibodies are scFv monoclonal antibodies. In some embodiments, the one or more antibodies are anti-MUC18 scFv monoclonal antibodies. In some embodiments of the methods and compositions disclosed herein, the anti-MUC18 antibody comprises a variable heavy chain amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO:2 or a fragment thereof. In some embodiments of the methods and compositions disclosed herein, the anti-MUC18 antibody comprises a variable light chain amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO:4 or a fragment thereof. In some embodiments of the methods and compositions disclosed herein, the variable heavy chain and the variable light chain of the anti-MUC18 antibody are linked by an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO:3 or a fragment thereof. In some embodiments, the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof. In some embodiments, the anti-MUC18 antibody comprises a variable heavy chain amino acid sequence comprising SEQ ID NO:2 or a fragment thereof. In some embodiments, the anti-MUC18 antibody comprises a variable light chain amino acid sequence comprising SEQ ID NO:4 or a fragment thereof. In some embodiments, the variable heavy chain and the variable light chain of the anti-MUC18 antibody are linked by an amino acid sequence comprising SEQ ID NO:3 or a fragment thereof. In some embodiments, the anti-MUC18 antibody binds to MUC18 or a binding region thereof expressed by sarcoma cells, carcinoma cells, or a combination thereof. In some embodiments, said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells. In some embodiments, said anti-MUC18 antibody binds to MUC18 or a binding region thereof expressed by carcinoma cells comprising melanoma cells or prostatic adenocarcinoma cells.

In some embodiments, the first or only CAR polypeptide does not comprise a CD3 zeta signaling domain. In some embodiments, the at least one costimulatory signaling region comprises the intracellular domain of a costimulatory molecule selected from the group consisting of CD28. 2B4, DNAM-1, 4-1BB, OX40, CD30, CD40, ICOS, lymphocyte function-associated antigen-1 (LFA-I), CD2, CD7, LIGHT, NKG2C, and any combination thereof. In some embodiments of the methods and compositions disclosed herein, the at least one costimulatory signaling region comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO:8, SEQ ID NO:9, SEQ ID NO: 10, SEQ ID NO:11, or a fragment thereof. In some embodiments, the at least one costimulatory signaling region comprises SEQ ID NO:8 or a fragment thereof. In some embodiments, the at least one costimulatory signaling region comprises SEQ ID NO:9 or a fragment thereof. In some embodiments, the at least one costimulatory signaling region comprises SEQ ID NO:10 or a fragment thereof. In some embodiments, the at least one costimulatory signaling region comprises SEQ ID NO:11 or a fragment thereof. In some embodiments, the one or more extracellular spacers comprise an IgG1 sequence, an IgG4, sequence, or a combination thereof. In some embodiments of the methods and compositions disclosed herein, the transmembrane domain comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO:7 or a fragment thereof. In some embodiments, the transmembrane domain comprises SEQ ID NO:7 or a fragment thereof.

In some embodiments of the methods and compositions disclosed herein, the first or only CAR polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 12, SEQ ID NO:13, SEQ ID NO:14, SEQ ID NO:15, or a fragment thereof. In some embodiments, the first or only CAR polypeptide comprises an amino acid sequence having at least about 80% sequence identity to SEQ ID NO:12 or a fragment thereof. In some embodiments, the first or only CAR polypeptide comprises an amino acid sequence having at least about 80% sequence identity to SEQ ID NO:13 or a fragment thereof. In some embodiments, the first or only CAR polypeptide comprises an amino acid sequence having at least about 80% sequence identity to SEQ ID NO: 14 or a fragment thereof. In some embodiments, the first or only CAR polypeptide comprises an amino acid sequence having at least about 80% sequence identity to SEQ ID NO:15 or a fragment thereof. In some embodiments, the first or only CAR polypeptide comprises SEQ ID NO: 12 or a fragment thereof. In some embodiments, the first or only CAR polypeptide comprises SEQ ID NO:13 or a fragment thereof. In some embodiments, the first or only CAR polypeptide comprises SEQ ID NO:14 or a fragment thereof. In some embodiments, the first or only CAR polypeptide comprises SEQ ID NO: 15 or a fragment thereof.

In some embodiments comprising the first or only CAR polypeptide, the composition further comprises a second CAR polypeptide comprising an NKG2D receptor or fragment thereof.

In some embodiments, the second CAR polypeptide comprises: the NKG2D receptor; a CD3 zeta signaling domain; and optionally, a detection molecule. In some embodiments, the extracellular domain of the NKG2D receptor binds to one or more NKG2D ligands comprising MICA, MICB, ULBP1, ULBP2, ULBP3, ULBP4, ULBP5, ULBP6, or a combination thereof. In some embodiments of the methods and compositions disclosed herein, the NKG2D receptor comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO:17 or a fragment thereof. In some embodiments, the NKG2D receptor comprises an amino acid sequence having at least about 80% sequence identity to SEQ ID NO:17 or a fragment thereof. In some embodiments, the NKG2D receptor comprises SEQ ID NO: 17 or a fragment thereof. In some embodiments of the methods and compositions disclosed herein, the CD3 zeta signaling domain comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 16 or a fragment thereof. In some embodiments, the CD3 zeta signaling domain comprises an amino acid sequence having at least about 80% sequence identity to SEQ ID NO:16 or a fragment thereof. In some embodiments, the CD3 zeta signaling domain comprises SEQ ID NO:16 or a fragment thereof. In some embodiments of the methods and compositions disclosed herein, the detection molecule comprises amino acid sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 18 and SEQ ID NO: 19 or fragments thereof. In some embodiments, the detection molecule comprises amino acid sequences having at least about 80% sequence identity to SEQ ID NO:18 and SEQ ID NO: 19 or fragments thereof. In some embodiments, the detection molecule comprises SEQ ID NO: 18 and SEQ ID NO:19 or fragments thereof. In some embodiments of the methods and compositions disclosed herein, the second CAR polypeptide comprises amino acid sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO:20 or fragments thereof. In some embodiments, the second CAR polypeptide comprises an amino acid sequence having at least about 80% sequence identity to SEQ ID NO:20 or a fragment thereof. In some embodiments, the second CAR polypeptide comprises SEQ ID NO:20 or a fragment thereof.

In some embodiments, the first or only and/or second CAR polypeptides are encoded by one or more isolated nucleic acid sequences. In some embodiments, the first or only and second CAR are encoded by the same isolated nucleic acid molecule. In some embodiments, the one or more isolated nucleic acid sequences are comprised in one or more expression vectors. In some embodiments, the one or more expression vectors are lentiviral vectors, gamma-retroviral vectors, adenoviral vectors, adeno-associated viral vectors, or a combination thereof. In some embodiments, the expression vector is comprised in a cell transfected with the expression vector, and the cell expresses a polypeptide encoded by the one or more isolated nucleic acid sequences comprised in the one or more expression vectors. In some embodiments, said cell is an immune effector cell. In some embodiments, the immune effector cell is selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof. In some embodiments, the immune effector cell is a Natural Killer (NK) cell. In some embodiments, the immune effector cells are transfected with one or more expression vectors comprising one or more isolated nucleic acid sequences encoding the first and/or second CAR polypeptides.

In some embodiments, expression of the first and second CAR polypeptides by an immune effector cell increases the likelihood of NK activation at tumor sites. In some embodiments, the immune effector cell promotes an immune response against a cancer when the antibody of the first CAR polypeptide binds MUC18 or a binding region thereof on a target cell and/or the extracellular domain of the NKG2D receptor of the second CAR polypeptide binds one or more NKG2D ligand binding targets on the target cell.

In some embodiments, the composition disclosed herein comprise a pharmaceutically acceptable carrier. In some embodiments, the amount of cells in the composition ranges from about 10⁴ up to about 10⁸ cells per kg body weight of the mammal.

In some embodiments, the cancer is a solid tumor cancer. In some embodiments, the solid tumor cancer is sarcoma, and wherein the sarcoma comprises rhabdomyosarcoma. Ewing sarcoma, clear cell sarcoma, or leiomyosarcoma. In some embodiments, the sarcoma is rhabdomyosarcoma. In some embodiments, the sarcoma is Ewing sarcoma. In some embodiments, the solid tumor cancer is carcinoma, and wherein the carcinoma comprises melanoma or prostatic adenocarcinoma. In some embodiments, the target cell population and/or tissue comprises solid tumor cancer cells and/or tissue. In some embodiments, the solid tumor cancer cells and/or tissue comprises sarcoma cells and/or tissue, carcinoma cells and/or tissue, or a combination thereof. In some embodiments, the sarcoma cells and/or tissue comprises rhabdomyosarcoma, Ewing sarcoma, clear cell sarcoma, or leiomyosarcoma cells and/or tissue. In some embodiments, the carcinoma cells and/or tissue comprises melanoma or prostatic adenocarcinoma cells and/or tissue.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise.” “consist essentially of.” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure.

It is contemplated that any embodiment discussed in this specification can be implemented with respect to any method or composition of the disclosure, and vice versa. Furthermore, compositions of the disclosure can be used to achieve methods of the disclosure.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1F show that NKG2D.ζ-NK cells expand and kill ligand-expressing targets. FIG. 1A, Schematic of SFG-based retroviral vector constructs for transduction of human NK cells. FIG. 1B, Human NK cells were expanded as described in Materials and Methods, and the percentage of CD56⁺/CD3⁻ NK cells at the time of retroviral transduction (day 4) is shown. Expanded NK cells (red circle) purified via depletion of CD3+ cells were transduced with NKG2D.ζ retroviral vector or empty vector control (referred to as “unmodified”), and transduction efficiencies are shown in the inset. FIG. 1C, NKG2D expression on NK cells (MFI, inset) was assessed with isotype antibody as control. Nontransduced NK cells exhibited similar NKG2D expression to empty vector-transduced NK cells. *, P=0.003 versus unmodified condition. FIG. 1D. Expression of NKG2D (absolute MFI on the y-axis) on NK cells from each donor (n=25) transduced with either empty vector or NKG2D.ζ construct was determined by flow cytometry. Each pair of data points connected by a line represent cells from a single donor, to confirm surface expression of the inventors' chimeric molecule after transduction. Black lines with gray block next to each group are mean MFI±SEM. FIG. 1E, NKG2D.ζ-NK cell cytotoxicity against K562 and LAN-1 tumor targets in a 4-hour 51Cr-release assay. Given that K562 and LAN-1 are both NK-sensitive targets, low E:T ratios were utilized to observe differences. Experiment is representative of at least three separate determinations from n=10 donors. *, P<0.01 versus unmodified NK cells at same E:T ratio. FIG. 1F, NKG2D.ζ-NK cells were expanded after transduction culture (as shown in schema), and fold expansion and cytotoxicity both pre- (day 7) and post- (day 17) secondary expansion were determined.

FIGS. 2A-2D show that transgenic NKG2D.ζ is unaffected by TGFβ or soluble NKG2D ligands. NKG2D.ζ or unmodified NK cells (n=5 donors) were cultured in the presence of TGFβ (5 ng/mL; FIGS. 2A, 2B) or the soluble NKG2D ligands MICA and MICB (FIGS. 2C, 2D) for 24, 48, and 72 hours. NKG2D receptor expression was determined by flow cytometry, and NK cytotoxicity against K562 targets was assessed in a 4-hour Cr-release assay at an 5:1 E:T ratio using 48-hour exposed NK cells. Viability of transduced NK cells after exposure to TGFβ for 24, 48, and 72 hours, as assessed by 7-AAD vital staining, was >90%. *, P=0.001 versus non-TGFβ/MICA-treated NK groups at same time points.

FIGS. 3A-3I show that human MDSCs express ligands for NKG2D and are killed by NKG2D.ζ-NK cells. FIG. 3A, NKG2D ligand expression on human MDSCs by flow cytometry. Immature dendritic cells (iDC) and mature DCs (mDC) were used as myeloid controls. T cells activated with CD3 and CD28 mAbs plus 100 IU/mL IL2 for 24 hours were used as lymphocyte control. LAN-1 and K562 cells were used as negative and positive controls, respectively. MFI of NKG2D ligand expression in parenthesis. Representative data from single donor (of n=25 normal donors). Isotype control for NKG2D staining routinely fell within the first log. FIG. 3B, NKG2D.ζ-NK cell cytotoxicity against autologous MDSCs as targets in a 4-hour 51Cr-release assay. In some wells of the cytotoxicity assay, a blocking mAb to NKG2D was added. Representative data from triplicate samples per data point from a single donor (of n=25 normal donors) are shown. *, P<0.01 versus unmodified NK cells at same E:T ratio. FIG. 3C, In the same experiment as FIG. 3B, the same batch of NKG2D.ζ-NK cells were analyzed for cytotoxicity against autologous B cells, monocytes, monocyte-derived iDC and mDC, and activated T cells (n=10 donors examined). FIG. 3D. M-MDSC frequency by flow cytometry from neuroblastoma tumor samples obtained from high-risk patients, as described in Materials and Methods. FIG. 3E, Cytotoxicity by NKG2D.ζ-NK cells derived from patient PBMC (harvested and frozen at the time of tumor sampling) against autologous tumor-derived MDSCs in a 4-hour 51Cr-release assay. Data shown are from triplicate samples per data point at a 10:1 E:T ratio. *, P<0.001 versus unmodified NK cells from the same donor. FIG. 3F, NKG2D.ζ-NK cells were cocultured with autologous MDSCs at 1:1 ratio plus low-dose 50 IU/mL IL2 to maintain NK survival, and fold change in the number of each cell type from the start of coculture was determined by flow cytometry at indicated time points. *, P<0.001 versus NK/MDSC fold change in unmodified NK cell cocultures. FIG. 3G, Cell-free supernatants were harvested from cocultures at day 3 and analyzed for IFNγ, TNFα, IL6, and IL10 by ELISA. #, P<0.01 versus corresponding cytokine in cocultures with unmodified NK cells. FIG. 3H, NKG2D ligand expression was determined for activated T cells (ATC) expressing NKG2D.ζ and NKG2D.ζ-NK cells. Expression of NKG2D ligands on nontransduced ATCs as control for T-cell activation. FIG. 3I, NKG2D.ζ-NK cells or NKG2D.ζ T cells were cocultured with autologous ATCs at 1:1 ratio and fold change in the number of each cell type from the start of coculture was determined by flow cytometry at indicated time points. *, P<0.001 versus ATC fold change at day 0 and 3 cocultures.

FIGS. 4A-4H show that NKG2D.ζ-NK cells eliminate intratumoral immunosuppressive cells and reduce tumor burden. (FIG. 4A) provides an overview of the experimental timeline. LAN-1 tumor cells, either alone (FIG. 4B) or admixed with human MDSCs (FIG. 4C), were injected s.c. in the flanks of NSG mice. When tumors reached a volume of approximately 100 mm³ (day 14, gray block arrow inset), no NK cells (PBS control), 1×10⁷ unmodified or NKG2D.ζ-NK cells were injected i.v., and tumor growth was measured over time via calipers. *, P<0.03 versus other conditions shown at the same time point. FIG. 4D, On day 26, intratumoral human MDSCs (CD33⁺, HLA-DR^{low}) were enumerated by flow cytometry and are presented as mean % MDSCs remaining per treatment group. **, P<0.005 versus unmodified NK treatment. FIG. 4E, Survival of groups by Kaplan-Meier analysis. #. P=0.024. Representative experiment of three performed. FIGS. 4F-4H, NKG2D.ζ-NK deplete intra-tumoral MDSC, M2, and Treg in vivo.

FIGS. 5A-5D show that NKG2D.ζ-NK cells secrete chemokines that recruit GD2.CAR-T cells. FIG. 5A. NKG2D.ζ or unmodified NK cells were cocultured with autologous MDSCs and cell-free culture supernatants harvested at 48 hours were analyzed for chemokines CXCL8, CCL5, CCL3, and CCL22 by ELISA. Shown are mean chemokine concentration±SEM for n=5 cocultures/donor (data from one of five representative donors are shown). *, P<0.005 versus unmodified NK cocultures. FIG. 5B, GD2.CAR-T cells were assayed for chemotaxis in Transwells (described in Materials and Methods) in response to supernatants derived from unmodified or NKG2D.ζ-NK cells cocultured with autologous MDSCs. Supernatants derived from monocyte (nonsuppressive myeloid cell control)-stimulated NK cells were also used. #, P<0.001 versus medium; **, P=0.009 versus “unmodified NK plus MDSCs” condition. FIG. 5C, LAN-1 tumor cells, alone or admixed with human MDSCs, were injected s.c. into the flank of NSG mice. When tumors reached a volume ˜100 mm³, 5×10⁶ GD2.CAR-T cells were injected i.v. alone on day 13 (GD2.CAR-T), or preceded by 5× 10⁶ NKG2D.ζ-NK cells i.v. injected on day 10 (chNK+GD2.CAR-T). GD2.CAR-T signal at tumor site was measured over time via live-animal bioluminescence imaging. FIG. 5D, Shown is mean±SEM (n=5 mice/group) bioluminescent signal of GD2.CAR-T cells expressed as radiance. *, P=0.01 versus all other groups.

FIGS. 6A-6D show that elimination of MDSCs by NKG2D.ζ-NK cells increases antitumor activity of GD2.CAR-T cells. Luciferase gene-transduced LAN-1 tumor cells, alone or admixed with human MDSCs, were injected s.c. into NSG mice. FIG. 6A, When tumors reached a volume ˜100 mm³, no treatment (No Tx; PBS control) or 5×10⁶ NKG2D.ζ-NK cells alone (chNK) were injected i.v. on day 10, and tumor growth was measured over time via live-animal bioluminescence imaging. Shown is mean±SEM (n=5 mice/group) bioluminescent signal expressed as radiance. #ns, P=0.18 versus no treatment (+MDSC) group. FIG. 6B, Survival of groups in A was determined by Kaplan-Meier analysis. #ns, P=0.059. FIG. 6C. In other groups of mice within the same experiment, 5×10⁶ GD2.CAR-T cells were injected i.v. alone on day 13 (GD2.CAR-T), or preceded by 5× 10⁶ NKG2D.ζ-NK cells injected on day 10 (chNK+GD2.CAR-T). *, P=0.001; #, ns; P=0.59 versus each other. FIG. 6D, Survival of groups in C by Kaplan-Meier analysis. Representative experiment of 5 separate experiments. *, P=0.002; **, P=0.001.

FIGS. 7A-7C provide a characterization of GD2.CAR-T cells. (FIG. 7A) The GD2 CAR construct is a 2nd generation CAR composed of a 14g2a scFv, a short IgG4 extracellular spacer, a CD28 transmembrane domain, and 4-1BB and CD3z endodomains, followed by IRES and a truncated NGFR sequence for detection. (FIG. 7B) Transduction efficiency (left histograms), CD4/CD8 composition (middle contour plots), and phenotype subsets of CD4 or CD8 T cells (right contour plots) of the GD2.CAR-T cell product generated for in vivo experiments was analyzed by flow cytometry. (FIG. 7C) Chemokine receptor expression on the GD2.CAR-T cell product generated for in vivo experiments by flow cytometry.

FIGS. 8A-8G illustrate the generation and characterization of human MDSCs. Human MDSCs were generated by culture of CD3⁻/CD25^{low} PBMC with IL-6 plus GM-CSF for 7 days, as per Methods. (FIG. 8A) Resultant population was analyzed for MDSC phenotypes of each subset of MDSCs as shown by flow cytometry. (FIG. 8B) MDSCs, LAN-1 tumor cells, and peripheral blood monocytes were cultured for 24 hours and cell-culture supernatants were assayed for the suppressive cytokines TGF-β, IL-6 and IL-10 by ELISA. *p<0.001 vs. both LAN-1 and Monocyte conditions. #p<0.03 vs. Monocyte condition. (FIG. 8C) PD-1 expression on ex vivo-generated mature dendritic cells, peripheral blood monocytes, and MDSCs was assessed by flow cytometry. (FIG. 8D) T-cell proliferation in the presence of MDSC or control myeloid cells was assessed using Cell Trace Violet membrane dye dilution analysis. Briefly, 1×10⁵ Cell Trace Violet-labeled autologous T cells (harvested at the time of MDSC generation) were plated in the presence of platebound CD3 and CD28 mAbs with 50 IU/mL IL-2 in the absence or presence of MDSCs or peripheral blood monocytes (as a myeloid cell control) at 1:1, 4:1 and 8:1 T cell:MDSC ratios. After 4 days of coculture, T cells were labeled with CD3 flow antibody and assessed for Cell Trace Violet dye dilution by flow cytometry. *p<0.01 vs. T cell alone condition. (FIG. 8E) Cell-free culture supernatants from (FIG. 8D) were assayed for IFN-g by ELISA. *p<0.01 vs. T cell alone condition. (FIG. 8F) GD2 antigen-directed CAR-T cells were co-cultured with GD2+ LAN-1 tumor cells at a 1:4 ratio with or without autologous MDSCs at 1 CART:1 MDSC ratio. Tumor was added again every 3 days as CAR antigen re-stimulation, and fold change in the number of GD2.CAR-T cells from the start of each co-culture was determined by flow cytometry at indicated time-points *p<0.001 vs. CAR-T cells with LAN-1 only condition. (FIG. 8G) Cell-free culture supernatants at day 3 from (FIG. 8F) were assayed for IFN-g. *p=0.02 vs. LAN-1+CAR-T+MDSC condition; #=0.005 vs. LAN-1+CAR-T condition.

FIGS. 9A-9D provide a characterization of an in vivo TME xenograft model. NSG mice were implanted subcutaneously in the dorsal left flank with 1×10⁶ Firefly luciferase (FfLuc)-expressing LAN-1 neuroblastoma cells admixed with 3×10⁵ ex vivo-generated MDSCs suspended in 100 μL of basement membrane Matrigel. (FIG. 9A) Tumor volumes were measured serially by caliper measurement and presented as tumor volume over time. (FIG. 9B) Peripheral blood was sampled on days 16 and 21 and assayed for suppressive cytokines human IL-10 and TGF-β by ELISA. Shown are results for day 16 (n=5 mice/group). #, *p<0.001 vs. same cytokine in “tumor only” mice. (FIG. 9C) On day 16, tumors were harvested, sectioned, and n=5 samples/section were analyzed for presence of microvasculature by H&E and human CD31 immunostaining (brownish red) and human MDSCs by S100A9 immunostaining (black). Shown are representative sections of tumors inoculated with or without MDSCs. (FIG. 9D) Number of S100A9+MDSCs within areas of each tumor section containing CD31+ vessels (CD31 pos.) were enumerated and compared to MDSC numbers in areas devoid of CD31+ vessels (CD31 neg.). *p=0.002 vs. tumors without MDSCs.

FIGS. 10A and 10B show NKG2D.ζ NK cells rescue defective GD2.CAR-T cell homing and infiltration into MDSC-containing tumors. (FIG. 10A) On day 32 of in vivo experiments reported in FIGS. 6A-6D, tumors were harvested, sectioned ex vivo to separate tumor periphery (outer ⅓ of tumor volume) vs. core (non-necrotic inner ⅔ of tumor volume), and n=5 samples/section were analyzed for presence of GD2.CAR-T cells by H&E and human CD3 immunostaining. Shown are representative sections of tumors from periphery vs. core of the treatment groups indicated showing CD3 staining (brown cells). (FIG. 10B) Number of CD3+ cells were enumerated and percent of the total number of GD2.CAR-T cells enumerated within tumors found in the periphery vs. core (y-axis) in each treatment group indicated (x-axis) from tumors with and without MDSCs is shown as Mean±SEM of n=5 sections/periphery or core, n=5 tumors/group. *p=0.02 vs. GD2.CAR-T infiltration into core of tumors without MDSC.

FIG. 11 shows infiltration of NKG2D.ζNK and GD2.CAR-T cells by IHC. GD2.CAR-T cells were injected three days after administration of NKG2D.ζ NK cells into mice bearing MDSC-containing tumors, as detailed in FIGS. 6A-6D and Example 1. On day 32, tumors from these mice were harvested, separated into sections containing tumor periphery from tumor core by blunt dissection, and sections from each tumor portion were analyzed for presence of human CD3⁺ (for GD2.CAR-T) and CD57⁺ (for human NK) cells by immunohistochemistry (IHC). Lack of CD57 expression on infused GD2.CAR-T was confirmed by flow cytometry prior to administration (not shown). Shown are absolute numbers of NKG2D.ζ-NK or GD2.CAR-T cells per high-powered field (HPF) at 40× magnification for each treatment condition: mice treated with NKG2D.ζ NK cells alone (NKG2D.ζ) GD2.CAR-T cell alone (GD2.CART) or the sequential combination. Shown is representative in vivo experiment of three performed with n=5 mice/group, as detailed in FIGS. 6A-6D legend.

FIGS. 12A and 12B show that sarcoma cell lines demonstrate expression of MUC18 and NKG2D ligand. (FIG. 12A) Rh4 (primary alveolar RMS), Rh41 (metastatic alveolar RMS), RD (embryonal RMS), and COG-E-352 (EWS) were stained for MUC18 expression with isotype shown in blue. (FIG. 12B) Rh4, Rh41, and RD cell lines were stained for pan-NKG2D ligand expression with recombinant human NKG2D Fc chimera protein.

FIGS. 13A and 13B show that NK cells are effectively transduced with a MUC18.CAR construct. (FIG. 13A) MUC18 cytotoxic CAR construct contains an IgG4 hinge and IgG1 short spacer in the extracellular portion. The intracellular portion contains a 4-1BB costimulatory domain and CD3z cytotoxic domain. NGFR serves as a detection molecule for transduction. (FIG. 13B) Transduction efficiency was assessed via detection of NGFR by flow cytometry within NK cells from two separate donors (shown), with transduction rates around 80%.

FIGS. 14A and 14B show that MUC18.CAR NK cells exhibit superior cytotoxicity against Rh4 compared to MUC18.CAR T cells. (FIG. 14A) MUC18.CAR NK cells and non-transduced NK cells were plated with chromium labeled Rh4 cells in a 4-hour cytotoxicity assay. (FIG. 14B) MUC18.CAR T cells and non-transduced T cells against Rh4 cells in similar cytotoxicity assay. MUC18.CAR T cells demonstrated low cytotoxic activity against Rh4. Non-transduced T cells did not kill Rh4.

FIGS. 15A-15D provide confirmation of specificity and function of the MUC18 scFV-based CAR molecule in primary human NK cells. (FIG. 15A) 293T cells transfected with full-length MUC18 (red) demonstrated MUC18 expression similar to Rh4 WT (blue). (FIG. 15B) MUC18.CAR NK cells demonstrated enhanced cytotoxicity against 293T-MUC18 transfected cells compared to their activity against 293T-WT cells that had low-level MUC18 expression. Unmodified NK cells had no killing against either the 293T-MUC18 or the 293T-WT cells. (FIG. 15C) MUC18 was genetically deleted from the Rh4 cell line using CRISPR-Cas9 with MUC18 expression shown (red) as compared to the Rh4 WT (blue). (FIG. 15D) MUC18.CAR NK cells demonstrated cytotoxicity at all E:T ratios against Rh4 WT, while MUC18.CAR NK and unmodified NK cells exhibited similarly low killing against Rh4-MUC18 KO.

FIGS. 16A-16D show that MUC18 co-stim.CAR NK cells proliferate but don't kill within suppressive sarcoma TME. (FIG. 16A) 4-1BB, OX40, 2B4, and DNAM1 co-stim. constructs. Novel co-culture assay as illustrated in (FIG. 16B) allows simultaneous interrogation by flow cytometry (FIG. 16C) of three TME cell types: M2 macrophages, Rh4 RMS, and NK cells. (FIG. 16D) Fold change in Rh4 (upper row) and in NK cells (lower row) over 6-day culture. White area in each graph is cell expansion; grey area is cell death. While MUC18 cytotoxic CAR NK (2nd column) killing and proliferation are muted in presence of M2s (red arrows), OX40- and DNAM1-costim.CAR NK cells proliferate in presence of M2 (green arrows) without additional killing compared to unmodified NK cells.

FIGS. 17A and 17B show that Rh4 xenografts containing MDSCs exhibit enhanced growth velocity and retain expression of MUC18 and NKG2D ligands. (FIG. 17A) Subcutaneous flank injections of Rh4 with (red box) or without (blue box) MDSCs was performed in NSG mice and tumor volumes were measured over time. (FIG. 17B) Tumors were harvested on day 43, digested en bloc ex vivo, and MUC18 and NKG2D ligand expression were assessed by flow cytometry. Representative mouse tumor samples from mouse M1 (Rh4 only) and mouse M4 (Rh4+MDSCs) are shown.

FIG. 18 shows that human NK cells co-expressing NKG2D.ζ and a MUC18 co-stim.CAR expand and kill M2-TAMs and rhabdomyosarcoma tumor. Primary human NK cells co-expressing NKG2D. and a MUC18 co-stim.CAR with OX40 endodomain were co-cultured with human rhabdomyosarcoma cells (Rh4) and M2-TAMs and percentage of all three cell types were enumerated by flow cytometry after 3 days of co-culture. Dual targeting NK cells simultaneously eliminated M2-TAMs as well as regressed Rh4 tumor (black arrows; bottom right), while NK cells expressing either CAR alone did not (blue arrows; bottom middle).

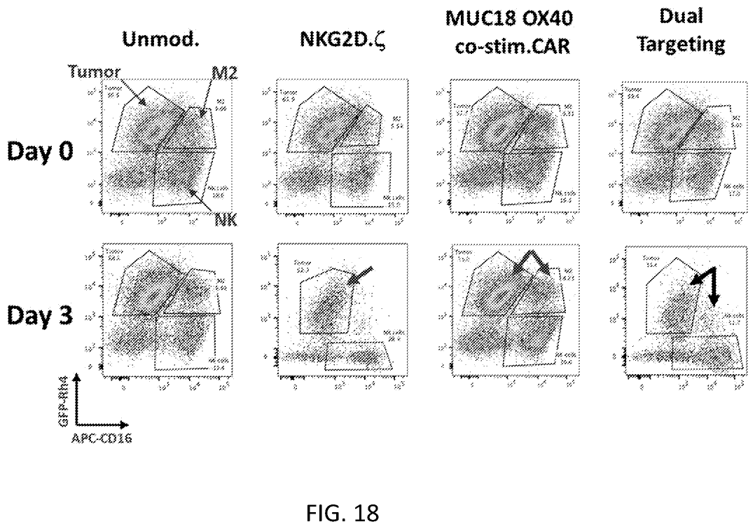

FIG. 19 illustrates a schema for a NK cell co-stim. approach. In the TME, where stress ligands for NKG2D are expressed on MDCSs and sarcoma cells, and MUC18 is expressed on tumor cells, NK cells co-expressing NKG2D.ζ and the MUC18.co-stim CAR will exhibit enhanced cytotoxicity and proliferation. In normal tissue, such as lung endothelium, which may express low levels of stress ligands and MUC18, but where self-MHC is highly expressed, modified NK cells may proliferate, however, no cytotoxic activity is expected.

### DETAILED DESCRIPTION

### I. Examples of Definitions

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the disclosure, for example. Some embodiments of the disclosure may consist of or consist essentially of one or more elements, method steps, and/or methods of the disclosure. It is contemplated that any method or composition described herein can be implemented with respect to any other method or composition described herein.

The term “allogeneic” refers to a cell or graft derived from a different animal of the same species. The term “xenogeneic” refers to a cell or graft derived from an animal of a different species.

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term“antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability.

Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen” refers to any substance that causes an immune system to produce antibodies against it, or to which a T cell responds. In some embodiments, an antigen is a peptide that is 5-50 amino acids in length or is at least, at most, or exactly 5, 10, 15, 20, 25, 30, 35, 40, 45, 50, 55, 60, 65, 70, 75, 80, 85, 90, 95, 100, 125, 150, 175, 200, 250, or 300 amino acids, or any derivable range therein. The term“antigen binding site” refers to a region of an antibody that specifically binds an epitope on an antigen.

The term “autologous” is meant to refer to any material derived from the same individual to which it is later to be re-introduced into the individual.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.).

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present disclosure, multiple epitopes can be recognized by a multispecific antibody.

The term “exogenous,” when used in relation to a protein, gene, nucleic acid, or polynucleotide in a cell or organism refers to a protein, gene, nucleic acid, or polynucleotide which has been introduced into the cell or organism by artificial means, or in relation a cell refers to a cell which was isolated and subsequently introduced to other cells or to an organism by artificial means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “fragment” refers to the fragment of an antibody comprising the constant domain of its heavy chain. The term“Fv fragment” refers to the fragment of an antibody comprising the variable domains of its heavy chain and light chain.

A “gene,” “polynucleotide,” “coding region,” “sequence.” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code. The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

As used herein, “isolated” for example, with respect to cells and/or nucleic acids means altered or removed from the natural state through human intervention.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide is particularly chimeric, i.e., composed of heterologous molecules.

The terms “peptide.” “protein,” and “polypeptide” are used interchangeably to refer to a natural or synthetic molecule comprising two or more amino acids linked by the carboxyl group of one amino acid to the alpha amino group of another. The term“pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or“fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologies. Thus, under designated conditions (e.g. immunoassay conditions in the case of an antibody), a specified ligand or antibody “specifically binds” to its particular“target” (e.g. an antibody specifically binds to an endothelial antigen) when it does not bind in a significant amount to other proteins present in the sample or to other proteins to which the ligand or antibody may come in contact in an organism. Generally, a first molecule that“specifically binds” a second molecule has an affinity constant (Ka) greater than about 10⁵ M-¹ (e.g., 10⁶ M-\10⁷ M-\10⁸ M-\10⁹ M-\10¹⁰ M-¹, 10¹¹ M-¹, and 10¹² M-¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician; treatment may or may not include correspondence through the internet. The subject may be of any gender, age, or race.

As used herein, the term “therapeutically effective amount” is synonymous with “effective amount”, “therapeutically effective dose”, and/or “effective dose” refers to an amount of an agent sufficient to produce a desired result or exert a desired influence on the particular condition being treated. In some embodiments, a therapeutically effective amount is an amount sufficient to inhibit or ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. Effective amount can also mean the amount of a compound, material, or composition comprising a compound of the present disclosure that is effective for producing some desired effect, e.g., treating or preventing cancer. The effective amount may vary depending on the organism or individual treated.

The appropriate effective amount to be administered for a particular application of the disclosed methods can be determined by those skilled in the art, using the guidance provided herein. For example, an effective amount can be determined experimentally using various techniques and/or extrapolated from in vitro and in vivo assays including dose escalation studies. Various concentrations of an agent may be used in preparing compositions incorporating the agent to provide for variations in the age of the patient to be treated, the severity of the condition, and/or the duration of the treatment and the mode of administration. One skilled in the art will recognize that the condition of the individual can be monitored throughout the course of therapy and that the effective amount of a compound or composition disclosed herein that is administered can be adjusted accordingly. Further, one of skill in the art recognizes that an amount may be considered effective even if the medical condition is not totally eradicated but improved partially. For example, the medical condition may be halted or reduced or its onset delayed, a side effect from the medical condition may be inhibited or partially reduced or completed eliminated, and so forth.

The terms “transformation” and “transfection” mean the introduction of a nucleic acid, e.g., an expression vector, into a recipient cell including introduction of a nucleic acid to the chromosomal DNA of said cell.

As used herein, the terms “treatment,” “treat.” or “treating” refers to intervention in an attempt to alter the natural course of the individual or cell being treated, and may be performed either for prophylaxis or during the course of pathology of a disease or condition, such as for example solid tumor cancers. Treatment may serve to accomplish one or more of various desired outcomes, including, for example, preventing occurrence or recurrence of disease, alleviation of symptoms, and diminishment of any direct or indirect pathological consequences of the disease, preventing disease spread, lowering the rate of disease progression, amelioration or palliation of the disease state, and remission or improved prognosis, and/or producing some desired effect.

The term“variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

A “vector” or “construct” (sometimes referred to as gene delivery or gene transfer “vehicle”) refers to a macromolecule, complex of molecules, or viral particle, comprising a polynucleotide to be delivered to a host cell, either in vitro or in vivo. The polynucleotide can be a linear or a circular molecule. A “plasmid”, a common type of a vector, is an extra-chromosomal DNA molecule separate from the chromosomal DNA which is capable of replicating independently of the chromosomal DNA. In certain cases, it is circular and double-stranded. By “expression construct” or “expression cassette” or “expression vector” is meant a nucleic acid molecule that is capable of directing transcription. An expression construct includes, at the least, a promoter or a structure functionally equivalent to a promoter. Additional elements, such as an enhancer, and/or a transcription termination signal, may also be included.

### II. Tumor Microenvironment

The TME comprises cancer-associated fibroblasts, microvasculature, and immune infiltrates partially consisting of immunosuppressive cells such as myeloid-derived suppressor cells (MDSCs), M2-TAMS, and regulatory T cells (Tregs).

MDSCs are a heterogenous population of cells of myeloid lineage progenitors that exert their immunosuppressive effects by multiple mechanisms including inhibition of T cell activation and cytotoxic activity, as well as induction of Tregs [16-17]. These cells are immature myeloid cells that fail to complete their maturation to macrophages, granulocytes, or DCs under chronic inflammatory conditions that are typical for the tumor microenvironment and that support tumor progression. Similar to other myeloid cells, MDSCs interact with other immune cell types including T cells, dendritic cells, macrophages and natural killer cells to regulate their functions. MDSCs are discriminated from other myeloid cell types in which they possess strong immunosuppressive activities rather than immunostimulatory properties.

MDSCs have been shown to derive from bone marrow hematopoietic precursors due to the altering of myelopoiesis by chronic inflammatory mediators such as granulocytemacrophage colony-stimulating factor (GM-CSF), granulocyte colony-stimulating factor (G-CSF), macrophage colonystimulating factor (M-CSF), stem cell factor (SCF), VEGF, IL-6, and IL-1β. These mediators are known to be produced both by tumor and stroma cells. Importantly, tumor cells have been reported to induce the production of inflammatory factors by tumor-infiltrating fibroblasts and immune cells, whereas stroma cells could further potentiate this production in tumor cells. These interactions form strong autocrine and paracrine loops supporting tumor growth and metastasis. A long-term secretion and accumulation of inflammatory mediators during chronic inflammation or tumor progression can result in the MDSC generation, expansion, and activation, leading to a profound immunosuppression.

Importantly, MDSCs acquire strong immunosuppressive capacities permitting them to inhibit antitumor reactivity of T and NK cells mediated by multiple mechanisms. Moreover, MDSCs are generated, recruited to the tumor site, and activated not only under the influence of soluble inflammatory mediators but also due to extracellular vesicles (EVs) secreted by tumor cells. EVs play a key role in the formation of MDSCs via the conversion of normal myeloid cells and altering the normal myelopoiesis. In addition, EVs help create a suitable microenvironment for the metastatic process.

MDSCs have been associated with disease progression, poor prognosis, and decreased effectiveness of immunotherapy [18-20]. Given their significant impact on promoting an inhibitory TME with clinical consequences, MDSCs represent an important target for weakening the TME of solid tumors.

MDSCs include some tumor associated macrophages (TAMs). TAMs are a central component in the strong link between chronic inflammation and cancer. TAMs are recruited to the tumor as a response to cancer-associated inflammation. Under the guidance of different microenvironmental signals, macrophages would polarize into two functional phenotypes, named as classically activated macrophages (M1) and alternatively activated macrophages (M2). Induced by lipopolysaccharide (LPS) or interferon C in vitro, M1 macrophages secrete nitric oxide, inflammatory factors, and chemokines, like IL-12, IL-23, MHC-II and B7 family members like B7-1 (CD80) and B7-2 (CD86), whose primary function is activating Th1 immune response to exert anti-tumor effect. The M2 pathway requires two types of signal molecules: the Th2 cytokine and the inducer of endogenous or exogenous tumor necrosis factor. Cytokines (IL-4, IL-13), vitamin D3, TGF-β, PGE2, and glucocorticoids are the main factors to induce M2 type activation41. Then M2 macrophages will secrete IL-1 chemokine receptor antagonists, matrix metalloproteinase (MMP), and upregulate the expression of MHC, restraining immune response and stimulating tumor invasion, growth and metastasis by secreting IL-10, TGF-β, etc., preventing T cells from effectively exerting anti-tumor effects. Thus, contrary to the anti-tumor effect of M1. M2 exerts anti-inflammatory and tumorigenic characters. In progressive tumor, M2 tumor-associated macrophages (TAMs) are in the majority, being vital regulators reacting upon TME.

Unlike normal macrophages, TAMs lack cytotoxic activity. TAMs have been induced in vitro by exposing macrophage progenitors to different immune regulatory cytokines, such as interleukin 4 (IL-4) and interleukin 13 (IL-13). TAMs gather in necrotic regions of tumors where they are associated with hiding cancer cells from normal immune cells by secreting interleukin 10 (IL-10), aiding angiogenesis by secreting vascular endothelial growth factor (VEGF) and nitric oxide synthase (NOS), supporting tumor growth by secreting epidermal growth factor (EGF) and remodeling the ECM. TAMs show sluggish NF-κB activation, which allows for the smoldering inflammation seen in cancer. TAMs secrete manifold growth factors to help oncogenesis; produce several proteolytic enzymes and motor-related proteins to support the invasion and metastasis of tumors; encode multiple gene products to promote angiogenesis; and are powerful manufacturers of numerous immunosuppressive molecules. An increased amount of TAMs is associated with worse prognosis and low survival rates in many human malignant neoplasms.

Regulatory T cells (Tregs) comprise diverse subsets of immunosuppressive cells that play critical roles in maintaining immune homeostasis and self-tolerance. They are also involved in controlling autoimmunity, infection, graft-versus-host disease, inflammation, fetal-maternal tolerance, and tumor immunity.

Tregs are broadly divided by lineage into thymic-derived tTregs, autoreactive T cells selected by high avidity interaction with self-antigens in the thymus, and peripheral pTregs, induced from naïve CD4⁺ T cells by sub-optimal antigen presentation in the periphery. tTregs are crucial for preserving self-tolerance and preventing autoimmunity, while pTregs maintain peripheral tolerance at mucosal interfaces and in response to external antigens. Both subsets of Tregs have traditionally been defined by expression of the Forkhead Box P3 (FoxP3) transcription factor—a “master regulator” of the suppressive lineage—and the IL-2 receptor a chain (CD25). pTregs additionally comprise two FoxP3″ subsets with important roles in oral tolerance: Tr1 and Th3 cells.

Tregs may exert their suppressive activity via a number of contact-dependent and independent mechanisms, including suppressive cytokines (TGF-β, IL-10, IL-35); immune checkpoints and inhibitory receptors (CTLA-4, PD-1, LAG-3, TIM-3, ICOS, TIGIT, IDO); direct cytotoxicity (perforin/granzyme-mediated); metabolic disruption of T effector cell activity (IL-2 consumption); and induction of tolerogenic DCs, which promote T cell exhaustion and expansion.

In cancers, Tregs are able to suppress anti-tumor immune responses and contribute to the development of an immunosuppressive tumor microenvironment (TME), thus promoting immune evasion and cancer progression. Tregs have been extensively characterized in the peripheral blood and immune infiltrates of different cancers. An accumulation of FoxP3⁺ Tregs and, in particular, a higher Treg:T effector cell (Teff) ratio within tumor tissue is associated with worse prognoses in many cancers, including ovarian cancer, pancreatic ductal adenocarcinoma, lung cancer, glioblastoma, non-Hodgkin's lymphoma, melanoma and other malignancies. The role of Tregs in immune escape is supported by clinical studies, and numerous in vitro studies, where Treg depletion released anti-tumor immunity. For example, transient Treg depletion induced regression of metastatic lesions in advanced stage melanoma patients. In breast cancer patients undergoing tumor resection and radiotherapy, Treg depletion prior to treatment is associated with an anti-tumor immune response and improved clinical outcomes. Additionally, Treg depletion followed by cancer antigen vaccination generated effective anti-tumor CD4⁺ and CD8⁺ T-cell responses in metastatic breast cancer patients.

Tregs are able to accumulate within the TME via several mechanisms. Tregs are recruited into tumors in response to chemokines secreted by tumor cells and innate immune cells; key chemokine-chemokine receptor combinations include CCL17/22-CCR4, CCL5-CCR5, CCL28-CCR10 and CXCL9/10/11-CXCR3. Tregs can be expanded in situ, and proliferate efficiently in response to tumor-derived factors (TGF-β, IL-10) within the TME. Generation of suppressive Tregs from non-suppressive CD25⁻ conventional T cells (Tconv) driven by tumor-derived transforming growth factor-beta (TGF-β) and adenosine; this has mainly been studied in murine models and the contribution of Treg induction to Treg accumulation within the TME in human cancer remains to be confirmed.

Tumor-infiltrating (TI) Tregs play direct roles in promoting immune evasion and the development of a pro-tumorigenic TME. They exhibit distinct phenotypic and functional profiles, upregulating markers associated with activation and enhanced suppressive activity. These include immune checkpoint molecules, cytotoxic T-lymphocyte associated protein 4 (CTLA-4), T-cell immunoglobulin and mucin-domain containing-3 (TIM-3/HAVCR2), lymphocyte activation gene-3 (LAG-3), programmed-death 1 (PD-1), inducible T-cell co-stimulator (ICOS), and glucocorticoid-induced TNFR family related gene (GITR); and T cell activation markers, CD25 and CD69. A number of the markers expressed on tumor-infiltrating Treg subsets are directly involved in suppressive function. Inhibitory immune checkpoint molecules, such as CTLA-4, PD-1, LAG-3 and TIM-3, act to dampen immune responses and prevent excessive T cell activation during physiological immune responses. CTLA-4 promotes T cell suppression by preferentially binding with CD80/86 signaling molecules over CD28, effectively blocking CD28 costimulatory signals required for T cell activation. Similarly, LAG-3, TIM-3 and PD-1 are inhibitory receptors that negatively regulate Teff and CD8+ cytotoxic lymphocyte (CTL) function, as well as potentially promoting Treg generation and function.

Neutrophils are polymorphonuclear immune cells that are critical components of the innate immune system. Neutrophils can accumulate in tumors and in some cancers, such as lung adenocarcinoma, their abundance at the tumor site is associated with worsened disease prognosis. When compared among 22 different tumor infiltrating leukocyte (TIL) subsets, neutrophils are especially important in diverse cancers, as illustrated by a meta-analysis of thousands of human tumors from various histologies (termed PRECOG). Neutrophil numbers (and myeloid cell precursors) in the blood can be increased in some patients with solid tumors. Experiments in mice have mainly shown that tumor-associated neutrophils exhibit tumor-promoting functions, but a smaller number of studies show that neutrophils can also inhibit tumor growth. Neutrophil phenotypes are diverse and distinct neutrophil phenotypes in tumors have been identified.

Tumor infiltrating lymphocytes (TILs) are lymphocytes that penetrate a tumor. TILs have a common origin with myelogenous cells at the hematopoietic stem cell, but diverge in development. Concentration is generally positively correlated. Cancer cells induce apoptosis of activated T cells (a class of lymphocyte) by secreting exosomes containing death ligands such as FasL and TRAIL, and via the same method, turn off the normal cytotoxic response of NK cells. This suggests that cancer cells actively work to restrain TILs.

### III. Immune Effector Cells

Disclosed herein are methods and compositions which utilize immune effector cells. In some embodiments, the immune effector cells are αβ-T cells, γδ-T cell, Natural Killer (NK) cells, Natural Killer T (NKT) cells, B cells, innate lymphoid cells (ILC), cytokine induced killer (CIK) cells, cytotoxic T lymphocytes (CTL), lymphokine activated killer (LAK) cells, and/or regulatory T cells.

The immune effector cells described herein may be engineered to express the disclosed CARs. These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells are human natural killer (NK) cells, and the NK cells are used in a manner that harnesses the power of their unique biology. NK cells, a lymphoid component of the innate immune system, produce MHC-unrestricted cytotoxicity and secrete proinflammatory cytokines and chemokines (10).

NK cells are CD56⁺/CD³⁻ large granular lymphocytes of the innate immune system which are involved in immune responses against viral infection or cells undergoing malignant transformation (8). Unlike T lymphocytes, NK cells do not require antigen sensitization or presentation by major histocompatibility complex (MHC) class I/II molecules to recognize their targets (9). Instead, the activation and cytotoxicity of NK cells is dependent on interactions between a multitude of cell-surface activating and inhibitory receptors (termed killer cell immunoglobulin-like receptors or KIR, and natural cytotoxicity receptors or NCR) and the ligands on the surface of target cells [2-3]. In other words, NK cell activation and cytotoxicity depends on the balance of activating versus inhibitory signals mediated by a multitude of cell-surface receptors engaging their ligands on target cells (9, 10). Of note, NK cells are potently inhibited or negatively regulated by receptors that bind self-HLA as a means of preventing autoimmunity (11). Overall, activation of NK cells upon encountering a target is governed by the proportion of engaged activating versus inhibitory receptors at the immunologic synapse [5]. Resting NK cells circulate in the peripheral blood and upon cytokine activation they are capable of extravasation and infiltration of most tissues which contain infected or malignant cells [6]. In addition, the constitutive surface expression of an activating, low-affinity receptor for antibody molecules (FcγRIIIa or CD16) enables NK cells to interact with antibody-coated cells and mediate antibody-dependent cellular cytotoxicity (ADCC).

In addition to direct cell cytotoxicity, NK cells secrete several immune-stimulatory cytokines such as interferon-gamma (IFN-γ) and tumor necrosis factor-alpha (TNF-α), as well as immune cell-recruiting chemokines such as RANTES, MIP1-α, MCP-1, and IL-8 that help coordinate and expand adaptive immune responses [7].

NK cells also modulate the activity of antigen-presenting myeloid cells within lymphoid organs, and recruit and activate effector T cells at sites of inflammation (11, 12). NK cells express NKG2D, a cytotoxicity receptor that is activated by nonclassic MHC molecules expressed on cells stressed by events such as DNA damage, hypoxia, or viral infection (13). NKG2D ligands are overexpressed on several solid tumors and on tumor-infiltrating MDSCs (14). NK cells, therefore, can alter the TME in favor of an antitumor response by eliminating suppressive elements such as MDSCs. However, the NKG2D cytotoxic adapter molecule DAP10 is downregulated by suppressive molecules of the TME, such as TGFβ (15), limiting the antitumor functions of NK cells.

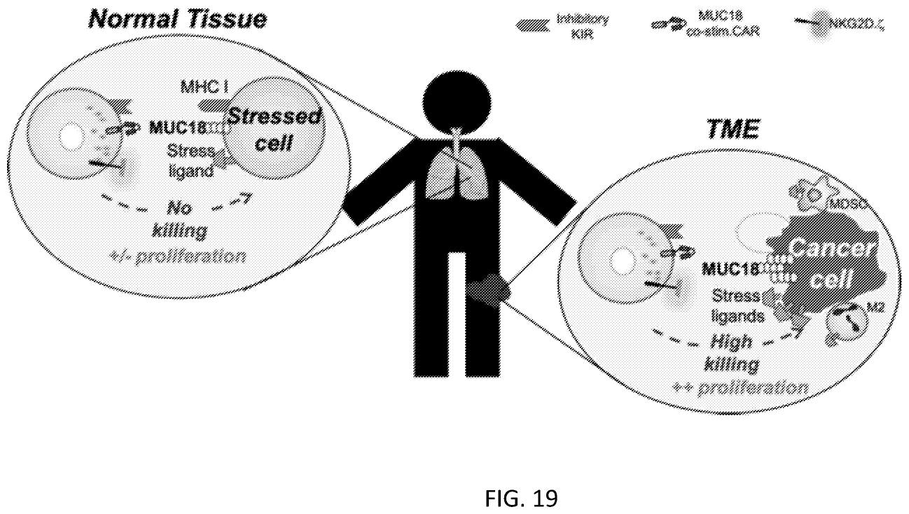

In some embodiments, provided is a cell-based immunotherapy that simultaneously targets the TME via NKG2D ligands expressed on immunosuppressive cells and MUC18, a sarcoma-associated antigen, specifically using NK cells as the platform because NK cells express inhibitory receptors for self-HLA and thus have endogenous “brakes” that inhibit toxicity against normal tissue. Using this unique receptor biology of NK cells, in some embodiments, NK cells are genetically engineered to co-express a NKG2D cytotoxic CAR and a CAR directed against MUC18 that provides only costimulatory signals to the NK cell, thus killing only in the presence of both antigens specifically within the TME (FIG. 19). In contrast, within normal tissue that might express the tumor-associated antigen, but where self-HLA is also expressed, the costimulatory signal by itself would be insufficient for NK activation, thereby preventing off-tumor toxicity (FIG. 19).

Using NK cells as a platform for NKG2D overexpression utilizes their cytotoxic and immune-modulatory potential while limiting toxicity. Recent studies have attempted to harness the unique biology of the NKG2D receptor against NKG2D ligand expressing tumors by overexpressing it in T lymphocytes (referred to as NKG2D-CAR T cells) [8, 9]. These T cell-based NKG2D CAR constructs contain costimulatory endodomains for enhanced T cell activation and co-express DAP10, an adaptor molecule that enhances the surface expression of NKG2D. Although these NKG2D-CAR T cells have shown activity against ligand-overexpressing tumors, lethal toxicity mediated by these NKG2D-CAR T cells has been recently reported, likely because NKG2D ligands are upregulated on stressed healthy tissues, including lung, liver, and GI [10]. In NK cells expressing an NKG2D CAR, positive signals from these ligands would be counteracted by inhibitory NK cell ligands on healthy tissues. However, since T cells do not express receptors for these inhibitory ligands, their NKG2D CAR can function unabated, thus leading to the severe toxicity observed. Thus, in some embodiments, NK cells which do not express the DAP10 molecule are used as the platform for an NKG2D-based CAR to limit toxicity while still allowing the full potential of NKG2D receptor function.

### IV. Chimeric Antigen Receptor Embodiments

Disclosed herein are methods and compositions which utilize chimeric antigen receptors (CARs) comprising CAR polypeptides, at least one CAR polypeptide comprising one or more antibodies or fragments thereof that binds to one or more cancer-associated antigens or a binding region thereof, and at least one CAR polypeptide comprising an NKG2D receptor or fragment thereof. These CARs can specifically recognize induced-self proteins which are completely absent or present only at low levels on surface of normal cells, but that are overexpressed by infected, transformed, senescent, and stressed cells.

Thus, some embodiments comprise methods and compositions for cell-based immunotherapies that simultaneously target the tumor microenvironment (TME) via NKG2D ligands and tumor cells via tumor-associated antigens, specifically using immune effector cells as the platform due to their reduced toxicity against normal tissue. In some embodiments, immune effector cells co-express an NKG2D cytotoxic CAR and a CAR directed against a tumor-associated antigen that provides costimulatory signals to the immune effector cell, thus killing only in the presence of both antigens specifically within the TME. In contrast, within normal tissue that might express the tumor-associated antigen, but where self-HLA is also expressed, the costimulatory signal by itself is insufficient for immune effector cell activation, thereby preventing off-tumor toxicity.

Immune effector cells, such Natural Killer (NK) cells, may be engineered to express these CARs. Therefore, also disclosed are methods for providing an immunotherapy in a subject using the disclosed immune effector cells. In some embodiments, expression of first and second CAR polypeptides by an immune effector cell increases the likelihood of immune effector cell activation at tumor sites, thereby increasing specificity and safety of the composition. In some embodiments, the immune effector cell promotes an immune response against a cancer when the antibody of a first CAR polypeptide binds MUC18 or a binding region thereof on a target cell and/or the extracellular domain of the NKG2D receptor of the second CAR polypeptide binds one or more NKG2D ligand binding targets on the target cell.

The term “chimeric antigen receptor” or “CAR” refers to engineered receptors, which graft an arbitrary specificity onto an immune effector cell. These receptors are used to graft the specificity of a monoclonal antibody onto a T cell; with transfer of their coding sequence facilitated by retroviral or lentiviral vectors. The receptors are called chimeric because they are composed of parts from different sources. The most common form of these molecules are fusions of single-chain variable fragments (scFv) derived from monoclonal antibodies, fused to CD3-zeta transmembrane and endodomain, CD28 or 41BB intracellular domains, or combinations thereof. Such molecules result in the transmission of a signal in response to recognition by the scFv of its target. An example of such a construct is 14g2a-Zeta, which is a fusion of a scFv derived from hybridoma 14g2a (which recognizes disialoganglioside GD2). When T cells express this molecule (as an example achieved by oncoretroviral vector transduction), they recognize and kill target cells that express GD2 (e.g. neuroblastoma cells). To target malignant B cells, investigators have redirected the specificity of T cells using a chimeric immunoreceptor specific for the B-lineage molecule, CD19. The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows the scFv to orient in different directions to enable antigen binding. The transmembrane domain is a typical hydrophobic alpha helix usually derived from the original molecule of the signaling endodomain which protrudes into the cell and transmits the desired signal.

In certain embodiments the chimeric antigen receptor (CAR) comprises at least one extracellular and at least one intracellular domain. An extracellular domain can comprise a target-specific binding element otherwise referred to as an antigen binding moiety that specifically binds to any particular antigen of interest. In specific embodiments, the extracellular domain comprises an antigen binding moiety that binds MUC18 or a fragment thereof. In various embodiments the target specific binding element comprise an anti-MUC18 antibody that binds MUC18 or a fragment thereof. The extracellular domain can comprise a target-specific binding element otherwise referred to as a ligand binding moiety that specifically binds to NKG2D or a fragment thereof bound by the NKG2D receptor or fragment thereof. In various embodiments the target specific binding element comprise an anti-MUC18 antibody, an NKG2D receptor, or fragments thereof.

In various embodiments the intracellular domain or otherwise the cytoplasmic domain comprises, one or more costimulatory signaling region(s), and in various embodiments, a zeta chain portion. The costimulatory signaling region refers to a portion of the CAR comprising the intracellular domain of a costimulatory molecule. In various embodiments costimulatory molecules are cell surface molecules other than antigen receptors or their ligands that are required for an efficient response of lymphocytes to antigen.

Between the extracellular domain and the transmembrane domain of the CAR, and/or between the cytoplasmic domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. In various embodiments the spacer domain may comprise up to 300 amino acids, or in various embodiments about 10 to about 100 amino acids, and in certain embodiments about 25 to about 50 amino acids.

In various embodiments a DNA construct comprising sequences of the one or more CARs disclosed herein are provided. In some embodiments, the one or more CARs are comprised in or encoded by the same construct, or isolated nucleic acid, while in others, the one or more CARs are comprises in separate constructs or isolated nucleic acids.

In some embodiments, the at least one CAR polypeptide comprising one or more antibodies or fragments thereof that bind to one or more cancer-associated antigens or a binding region thereof comprises an anti-MUC18 antibody or fragments thereof that bind to MUC18 or a binding region thereof, one or more hinges or spacers, a transmembrane domain, one or more cytoplasmic signaling domains, and one or more detection domains and/or molecules. In some embodiments, the at least one CAR polypeptide comprising one or more antibodies or fragments thereof that binds to one or more cancer-associated antigens or a binding region thereof comprises an anti-MUC18 antibody or fragments thereof that bind to MUC18 or a binding region thereof, an IgG4 hinge, an IgG1 spacer, a CD28 transmembrane domain, a CD35 cytoplasmic signaling domain, an IRES domain, and an NGFR detection molecule, or a combination of the foregoing.

In some embodiments, the at least one CAR polypeptide comprising one or more antibodies or fragments thereof that binds to one or more cancer-associated antigens or a binding region thereof comprises one or more of the following sequences:

CD8 leader sequence:
(SEQ ID NO: 1)
MALPVTALLLPLALLLHAARP
MUC18 scFv heavy chain sequence:
(SEQ ID NO: 2)
QVQLQESGPGLVKPSETLSLTCTVSGGSISSYYWSWIRQPPGKGL
EWIGYIYYTWTSNYNPSLKSRVTISVDTSKNQFSLRLSSVTAADT
AVYYCARDQGQWLLPDAFDIWGQGTMVTVSS
Linker sequence:
(SEQ ID NO: 3)
GGGGSGGGGGGGGS
MUC18 scFv light chain sequence:
(SEQ ID NO: 4)
DIVMTQSPLSLPVTPGEPASISCRSSQSLLRSNGYNYLDWYLQKP
GQSPHLLIYLGSNRASGVPDRESGSGSGTDFTLKISRVEAEDVGV
YYCMQAQQSPITFGQGTRLEIK
IgG4 short hinge sequence:
(SEQ ID NO: 5)
ESKYGPPCPPCP
IgG1 CH3 domain sequence:
(SEQ ID NO: 6)
GQPREPQVYTLPPSRDELTKNQVSLTCLVKGFYPSDIAVEWESNG
QPENNYKTTPPVLDSDGSFFLYSKLTVDKSRWQQGNVFSCSVMHE
ALHNHYTQKSLSLSPGK
CD28 transmembrane domain sequence:
(SEQ ID NO: 7)
FWVLVVVGGVLACYSLLVTVAFIIFWVRS
2B4 endodomain sequence:
(SEQ ID NO: 8)
WRRKRKEKQSETSPKEFLTIYEDVKDLKTRRNHEQEQTFPGGGST
IYSMIQSQSSAPTSQEPAYTLYSLIQPSRKSGSRKRNHSPSFNST
IYEVIGKSQPKAQNPARLSRKELENFDVYS
41BB-L endodomain sequence:
(SEQ ID NO: 9)
KRGRKKLLYIFKQPFMRPVQTTQEEDGCSCRFPEEEEGGCEL
DNAM-1 endodomain sequence:
(SEQ ID NO: 10)
NRRRRRERRDLFTESWDTQKAPNNYRSPISTSQPTNQSMDDTRED
IYVNYPTFSRRPKTRV
OX40 endodomain:
(SEQ ID NO: 11)
RDQRLPPDAHKPPGGGSFRTPIQEEQADAHSTLAKI

In some embodiments, the at least one CAR polypeptide comprising one or more antibodies or fragments thereof that binds to one or more cancer-associated antigens or a binding region thereof comprises one or more of the following sequence:

(SEQ ID NO: 12)
MALPVTALLLPLALLLHAARPQVQLQESGPGLVKPSETLSLTCTV
SGGSISSYYWSWIRQPPGKGLEWIGYIYYTWTSNYNPSLKSRVTI
SVDTSKNQFSLRLSSVTAADTAVYYCARDQGQWLLPDAFDIWGQG
TMVTVSSGGGGSGGGGSGGGGSDIVMTQSPLSLPVTPGEPASISC
RSSQSLLRSNGYNYLDWYLQKPGQSPHLLIYLGSNRASGVPDRFS
GSGSGTDFTLKISRVEAEDVGVYYCMQAQQSPITFGQGTRLEIKE
SKYGPPCPPCPGQPREPQVYTLPPSRDELTKNQVSLTCLVKGFYP
SDIAVEWESNGQPENNYKTTPPVLDSDGSFFLYSKLTVDKSRWQ
QGNVFSCSVMHEALHNHYTQKSLSLSPGKFWVLVVVGGVLACYSL
LVTVAFIIFWVRSWRRKRKEKQSETSPKEFLTIYEDVKDLKTRRN
HEQEQTFPGGGSTIYSMIQSQSSAPTSQEPAYTLYSLIQPSRKSG
SRKRNHSPSFNSTIYEVIGKSQPKAQNPARLSRKELENFDVYS

In some embodiments, the at least one CAR polypeptide comprising one or more antibodies or fragments thereof that binds to one or more cancer-associated antigens or a binding region thereof comprises one or more of the following sequence:

(SEQ ID NO: 13)
MALPVTALLLPLALLLHAARPQVQLQESGPGLVKPSETLSLTCTV
SGGSISSYYWSWIRQPPGKGLEWIGYIYYTWTSNYNPSLKSRVTI
SVDTSKNQFSLRLSSVTAADTAVYYCARDQGQWLLPDAFDIWGQG
TMVTVSSGGGGSGGGGSGGGGSDIVMTQSPLSLPVTPGEPASISC
RSSQSLLRSNGYNYLDWYLQKPGQSPHLLIYLGSNRASGVPDRES
GSGSGTDFTLKISRVEAEDVGVYYCMQAQQSPITFGQGTRLEIKE
SKYGPPCPPCPGQPREPQVYTLPPSRDELTKNQVSLTCLVKGFYP
SDIAVEWESNGQPENNYKTTPPVLDSDGSFFLYSKLTVDKSRWQ
QGNVFSCSVMHEALHNHYTQKSLSLSPGKFWVLVVVGGVLACYSL
LVTVAFIIFWVRSKRGRKKLLYIFKQPFMRPVQTTQEEDGCSCRF
PEEEEGGCEL

In some embodiments, the at least one CAR polypeptide comprising one or more antibodies or fragments thereof that binds to one or more cancer-associated antigens or a binding region thereof comprises one or more of the following sequence:

(SEQ ID NO: 14)
MALPVTALLLPLALLLHAARPQVQLQESGPGLVKPSETLSLTCTV
SGGSISSYYWSWIRQPPGKGLEWIGYIYYTWTSNYNPSLKSRVTI
SVDTSKNQFSLRLSSVTAADTAVYYCARDQGQWLLPDAFDIWGQG
TMVTVSSGGGGSGGGGSGGGGSDIVMTQSPLSLPVTPGEPASISC
RSSQSLLRSNGYNYLDWYLQKPGQSPHLLIYLGSNRASGVPDRES
GSGSGTDFTLKISRVEAEDVGVYYCMQAQQSPITFGQGTRLEIKE
SKYGPPCPPCPGQPREPQVYTLPPSRDELTKNQVSLTCLVKGFYP
SDIAVEWESNGQPENNYKTTPPVLDSDGSFFLYSKLTVDKSRWQQ
GNVFSCSVMHEALHNHYTQKSLSLSPGKFWVLVVVGGVLACYSLL
VTVAFIIFWVRSNRRRRRERRDLFTESWDTQKAPNNYRSPISTSQ
PTNQSMDDTREDIYVNYPTFSRRPKTRV

In some embodiments, the at least one CAR polypeptide comprising one or more antibodies or fragments thereof that binds to one or more cancer-associated antigens or a binding region thereof comprises one or more of the following sequence:

(SEQ ID NO: 15)
MALPVTALLLPLALLLHAARPQVQLQESGPGLVKPSETLSLTCTV
SGGSISSYYWSWIRQPPGKGLEWIGYIYYTWTSNYNPSLKSRVTI
SVDTSKNQFSLRLSSVTAADTAVYYCARDQGQWLLPDAFDIWGQG
TMVTVSSGGGGSGGGGSGGGGSDIVMTQSPLSLPVTPGEPASISC
RSSQSLLRSNGYNYLDWYLQKPGQSPHLLIYLGSNRASGVPDRES
GSGSGTDFTLKISRVEAEDVGVYYCMQAQQSPITFGQGTRLEIKE
SKYGPPCPPCPGQPREPQVYTLPPSRDELTKNQVSLTCLVKGFYP
SDIAVEWESNGQPENNYKTTPPVLDSDGSFFLYSKLTVDKSRWQQ
GNVFSCSVMHEALHNHYTQKSLSLSPGKFWVLVVVGGVLACYSLL
VTVAFIIFWVRSRDQRLPPDAHKPPGGGSFRTPIQEEQADAHSTL
AKI

In some embodiments, the at least one CAR polypeptide comprising an NKG2D receptor or fragment thereof comprises an NKG2D receptor, one or more cytoplasmic signaling domains, and one or more detection domains and/or molecules. In some embodiments, the at least one CAR polypeptide comprising an NKG2D receptor or fragment thereof comprises an NKG2D receptor, a CD32 cytoplasmic signaling domain, an IRES-tCD19 detection molecule, or a combination of the foregoing.

In some embodiments, the at least one CAR polypeptide comprising an NKG2D receptor or fragment thereof comprises the following sequences:

CD3ζ signaling domain sequence:
(SEQ ID NO: 16)
MRVKFSRSAEPPAYQQGQNQLYNELNLGRREEYDVLDKRRGRDPE
MGGKPRRKNPQEGLYNELQKDKMAEAYSEIGMKGERRRGKGHDGL
YQGLSTATKDTYDALHMQALPPR
NKG2D intracellular, transmembrane, and
extracellular domains sequence:
(SEQ ID NO: 17)
GWIRGRRSRHSWEMSEFHNYNLDLKKSDFSTRWQKQRCPVVKSKC
RENASPFFFCCFIAVAMGIRFIIMVAIWSAVFLNSLFNQEVQIPL
TESYCGPCPKNWICYKNNCYQFFDESKNWYESQASCMSQNASLLK
VYSKEDQDLLKLVKSYHWMGLVHIPTNGSWQWEDGSILSPNLLTI
IEMQKGDCALYASSFKGYIENCSTPNTYICMQRTV
IRES sequence:
(SEQ ID NO: 18)
YVNSAPLPPPPLTLLAEAAWNKAGVRLSICYFPPYCRLLAMGPGN
LALSSRAFLGVFPLSPKECKVCMSRKQFLWKLLEDKQRLRPFAGS
GTPHLATGASAAKSHVYKIHLQRRHNPSATLVGLWKESNGSPQAY
STRGRMPRRYPIVWDLIWGLGAHALHVFSRGKTSRPPEPRGRGFP
LKNTII
tCD19 sequence:
(SEQ ID NO: 19)
MPPPRLLFFLLFLTPMEVRPEEPLVVKVEEGDNAVLQCLKGTSDG
PTQQLTWSRESPLKPFLKLSLGLPGLGIHMRPLAIWLFIFNVSQQ
MGGFYLCQPGPPSEKAWQPGWTVNVEGSGELFRWNVSDLGGLGCG
LKNRSSEGPSSPSGKLMSPKLYVWAKDRPEIWEGEPPCLPPRDSL
NQSLSQDLTMAPGSTLWLSCGVPPDSVSRGPLSWTHVHPKGPKSL
LSLELKDDRPARDMWVMETGLLLPRATAQDAGKYYCHRGNLTMSF
HLEITARPVLWHWLLRTGGWKVSAVTLAYLIFCLCSLVGILHLQR
ALVLRRKRKRMTDPTRRF

In some embodiments, the at least one CAR polypeptide comprising an NKG2D receptor or fragment thereof comprises the following sequence:

(SEQ ID NO: 20)
MRVKFSRSAEPPAYQQGQNQLYNELNLGRREEYDVLDKRRGRDPE
MGGKPRRKNPQEGLYNELQKDKMAEAYSEIGMKGERRRGKGHDGL
YQGLSTATKDTYDALHMQALPPRGWIRGRRSRHSWEMSEFHNYNL
DLKKSDFSTRWQKQRCPVVKSKCRENASPFFFCCFIAVAMGIRFI
IMVAIWSAVFLNSLFNQEVQIPLTESYCGPCPKNWICYKNNCYQF
FDESKNWYESQASCMSQNASLLKVYSKEDQDLLKLVKSYHWMGLV
HIPTNGSWQWEDGSILSPNLLTIIEMQKGDCALYASSFKGYIENC
STPNTYICMQRTVYVNSAPLPPPPLTLLAEAAWNKAGVRLSICYF
PPYCRLLAMGPGNLALSSRAFLGVFPLSPKECKVCMSRKQFLWKL
LEDKQRLRPFAGSGTPHLATGASAAKSHVYKIHLQRRHNPSATLV
GLWKESNGSPQAYSTRGRMPRRYPIVWDLIWGLGAHALHVFSRGK
TSRPPEPRGRGFPLKNTIIMPPPRLLFFLLFLTPMEVRPEEPLVV
KVEEGDNAVLQCLKGTSDGPTQQLTWSRESPLKPFLKLSLGLPGL
GIHMRPLAIWLFIFNVSQQMGGFYLCQPGPPSEKAWQPGWTVNVE
GSGELFRWNVSDLGGLGCGLKNRSSEGPSSPSGKLMSPKLYVWAK
DRPEIWEGEPPCLPPRDSLNQSLSQDLTMAPGSTLWLSCGVPPDS
VSRGPLSWTHVHPKGPKSLLSLELKDDRPARDMWVMETGLLLPRA
TAQDAGKYYCHRGNLTMSFHLEITARPVLWHWLLRTGGWKVSAVT
LAYLIFCLCSLVGILHLQRALVLRRKRKRMTDPTRRF

Also disclosed is dual CAR immune effector cell containing the disclosed NKG2D CAR, and at least one other CAR with a different ligand binding target. In these embodiments, one CAR can include only the CD33 domain and the other CAR can include only the costimulatory domain(s). In these embodiments, dual CAR immune effector cell activation would require co-expression of both targets on the target cell.

Therefore, in some embodiments, the disclosed NKG2D CAR polypeptide contains an incomplete endodomain. For example, the CAR polypeptide can contain only an intracellular signaling domain. In these embodiments, the immune effector cell is not activated unless it and a second CAR polypeptide that contains a costimulatory signaling region both bind their respective targets. The disclosed dual CAR immune effector cell can contain the disclosed NKG2D CAR and at least one other CAR with a different ligand binding target, such as MUC18 or another tumor-associated antigen.

Also disclosed are bi-specific CARs that contain NKG2D extracellular, ecto, domain and an scFv that binds at least other target antigen, such as MUC18 or other tumor-associated antigen. Also disclosed are CARs designed to work only in conjunction with another CAR that binds a different antigen, such as MUC18 or other tumor-associated antigen. For example, in these embodiments, the intracellular, or endo, domain of the disclosed CAR may contain only a signaling domain. The second CAR provides the missing signal if it is activated. For example, if the disclosed CAR contains a signaling domain, then the immune effector cell containing this CAR is only activated if another CAR containing a costimulatory signaling domain binds its respective antigen or ligand. Likewise, if the disclosed CAR contains a costimulatory signaling domain but not a signaling domain, then the immune effector cell containing this CAR is only activated if another CAR containing a signaling domain binds its respective antigen or ligand.

Tumor antigens are proteins that are produced by tumor cells that elicit an immune response. The additional antigen binding domain can be an antibody or a natural ligand of the tumor antigen. The selection of the additional antigen binding domain will depend on the particular type of cancer to be treated.

Non-limiting examples of tumor antigens other than MUC18 that may be targeted include at least the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, MUC18, LMP1, EBMA-1, BARF-1, CS1, CD319, HER1, B7H6, L1 CAM, IL6, and MET. Tumor antigens include, for example, a glioma-associated antigen, carcinoembryonic antigen (CEA), EGFRvlll, IL-IIRa, IL-13Rα, EGFR, FAP, B7H3, Kit, CA LX, CS-1, MUC1, BCMA, bcr-abl, HER2, b-human chorionic gonadotropin, alphafetoprotein (AFP), ALK, CD19, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-1a, LMP2, NCAM, p53, p53 mutant, Ras mutant, gplOO, prostein, OR51 E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1.MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, mesothelin, and any combination thereof.

### A. Anti-MUC18 Antibody

In some embodiments, the at least one CAR polypeptide comprises an anti-MUC18 antibody or fragments thereof that bind to MUC18 or a binding region thereof.

A significant challenge in designing immunotherapies for solid tumors is identification of ideal tumor-associated antigens (TAA) or targets. Given the narrow mutation landscape in sarcomas, for example, neoantigens secondary to gene mutations are predicted to be rare [4]. The Pediatric Oncology Branch of the National Cancer Institute (NCI) performed global gene expression profiling of several pediatric cancers, including alveolar rhabdomyosarcoma (ARMS), embryonal rhabdomyosarcoma (ERMS) and Ewing sarcoma (EWS), and compared these profiles to normal tissue databases in order to identify cell surface proteins that may serve as immunotherapy targets [13]. Gene expression levels from individual tumor samples were averaged and compared to average normal tissue expression levels, allowing for a statistical measure of difference of a specific transcript's expression from its expression in normal tissues. A T-statistic was generated for each gene's expression level, with higher T values representing increased difference from normal tissues; T values ranged from 5.86-27.68 for the 12 tumor types included. For ARMS and ERMS, melanoma cellular adhesion molecule (MCAM/MUC18) was the most highly expressed tumor antigen identified, with T values of 27.68 and 20.87, respectively [13]. EWS also expressed MCAM, though at a lower level, with a T value of 9.27 [13].

Melanoma cellular adhesion molecule (MCAM), also known as MUC18, CD146, cell surface glycoprotein P1H12, cell surface glycoprotein MUC18, melanoma-associated antigen A32, and S-endo 1 endothelial-associated antigen, is a 113 kDa transmembrane glycoprotein encoded by the MCAM gene that belongs to the immunoglobulin superfamily and functions as a calcium-independent adhesion molecule [14-15]. Two isoforms exist (MCAM long (MCAM-1), and MCAM short, or MCAM-s) which differ in the length of their cytoplasmic domain. Activation of these isoforms seems to produce functional differences as well. Natural killer cells transfected with MCAM-1 demonstrate decreased rolling velocity and increased cell adhesion to an endothelial cell monolayer and increased microvilli formation while cells transfected with MCAM-s showed no change in adhesion characteristics.

MUC18 is a mediator of several intracellular signaling mechanisms. MUC18 has been studied extensively in malignant melanoma and its expression in melanoma cell lines has been shown to correlate with their ability to grow and produce metastases in vivo [14]. A fully humanized anti-MCAM/MUC18 antibody (ABX-MA1) has been evaluated in pre-clinical studies targeting melanoma and osteosarcoma. Mice with melanoma treated with ABX-MA1 developed small tumors at the injection sites and fewer lung metastases than control IgG treated mice [15]. In osteosarcoma, the incidence of spontaneous lung metastases was significantly lower in mice treated with ABX-MA1 compared to IgG-treated control mice [14].

Similar to the majority of TAAs currently being studied in solid tumors, MUC18 expression is not limited to malignant tissues, as it is also expressed at low levels on several normal adult tissues including smooth muscle, endothelium, mammary ductal, lobular epithelium, and peripheral nerve tissue [14-15]. In fact, the majority of tumor-associated antigens in sarcomas are also expressed at low levels in normal tissues. Given the expression of MUC18 on endothelium, mammary ductal epithelium, and peripheral nerves, utilizing it as a target for cellular immunotherapy presents a significant risk for off-target toxicity which must be addressed. A potential approach to limit off-target toxicity includes genetically engineering immunotherapy cells with restricted activation specifically within the tumor microenvironment (TME).

In some embodiments of the disclosure, there are compositions comprising CARs which encompass a nucleotide and/or amino acid sequence of an antibody which binds to MUC18 or fragments thereof. As a reference sequence, a MUC18 polypeptide sequence is in the UniProtKB database at Accession Number P43121:

P43121
(SEQ ID NO: 21)
MGLPRLVCAFLLAACCCCPRVAGVPGEAEQPAPELVEVEVGSTAL
LKCGLSQSQGNLSHVDWFSVHKEKRTLIFRVRQGQGQSEPGEYEQ
RLSLQDRGATLALTQVTPQDERIFLCQGKRPRSQEYRIQLRVYKA
PEEPNIQVNPLGIPVNSKEPEEVATCVGRNGYPIPQVIWYKNGRP
LKEEKNRVHIQSSQTVESSGLYTLQSILKAQLVKEDKDAQFYCEL
NYRLPSGNHMKESREVTVPVFYPTEKVWLEVEPVGMLKEGDRVEI
RCLADGNPPPHFSISKQNPSTREAEEETTNDNGVLVLEPARKEHS
GRYECQGLDLDTMISLLSEPQELLVNYVSDVRVSPAAPERQEGSS
LTLTCEAESSQDLEFQWLREETGQVLERGPVLQLHDLKREAGGGY
RCVASVPSIPGLNRTQLVNVAIFGPPWMAFKERKVWVKENMVLNL
SCEASGHPRPTISWNVNGTASEQDQDPQRVLSTLNVLVTPELLET
GVECTASNDLGKNTSILFLELVNLTTLTPDSNTTTGLSTSTASPH
TRANSTSTERKLPEPESRGVVIVAVIVCILVLAVLGAVLYFLYKK
GKLPCRRSGKQEITLPPSRKSELVVEVKSDKLPEEMGLLQGSSGD
KRAPGDQGEKYIDLRH

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric.” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

In some embodiments, the anti-MUC18 antibody or fragments thereof that bind to MUC18 or a binding region thereof comprise a single-chain variable fragment (scFv) derived from a MUC18 antibody. “Single-chain Fv” or “scFv” antibody fragments comprise the V_{H} and V_{L} domains of an antibody, wherein these domains are present in a single polypeptide chain. In some embodiments, the V_{H} of the MUC18 antibody comprises SEQ ID NO:2. In some embodiments, the V_{L} of the MUC18 antibody chain comprises SEQ ID NO:4. In some embodiments, the antigen-binding domain further comprises a peptide linker between the V_{H} and V_{L} domains, which may facilitate the scFv forming the desired structure for antigen binding. In some embodiments, the peptide linker between the V_{H} and V_{L} domains of the MUC18 antibody comprises SEQ ID NO:3.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

The binding affinity of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M. In some embodiments, the K_{D} of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M (or any derivable range therein).

Binding affinity, K_{A}, or K_{D} can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some embodiments, the anti-MUC18-antibody is humanized. In some embodiments, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity or expression level in host cells, compared to a polypeptide comprising a non-humanized binding region, such as a binding region from a mouse.

In some embodiments, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some embodiments, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

### B. NKG2D Receptor

In some embodiments, the at least one CAR polypeptide comprises an NKG2D receptor or fragment thereof.

NKG2D, also known as KLRK1, CD134, natural killer group 2D, and killer cell lectin-like receptor K1, is a transmembrane protein belonging to the CD94/NKG2 family of C-type lectin-like receptors. NKG2D is encoded by KLRK1 gene, and in humans, it is expressed by NK cells, γδ T cells and CD8+αβ T cells. NKG2D recognizes induced-self proteins from MIC and RAET1/ULBP families which appear on the surface of stressed, malignant transformed, and infected cells.

Human NKG2D receptor complex assembles into a hexameric structure. NKG2D itself forms a homodimer whose ectodomains serve for ligand binding. In unmodified cells, each NKG2D monomer is associated with DAP10 dimer. This association is maintained by ionic interaction of a positively charged arginine present in a transmembrane segment of NKG2D and negatively charged aspartic acids within both transmembrane regions of DAP10 dimer. DAP10, also known as hematopoietic cell signal transducer, is encoded by the HCST gene and functions as an adaptor protein and transduces the signal after the ligand binding by recruiting the p85 subunit of PI3K and Grb2-Vav1 complex which are responsible for subsequent downstream events.

NKG2D ligands are induced-self proteins which are completely absent or present only at low levels on surface of normal cells, but they are overexpressed by infected, transformed, senescent and stressed cells. Induced-self proteins are markers of abnormal self, which can be recognized upon infected (in particular, virus-infected) and transformed cells. Therefore, the recognition of “induced self” is an important strategy for surveillance of infection or tumor transformation; it results in elimination of the affected cells by activated NK cells or other immunological mechanisms. Similarly, γδ T cells can recognize induced-self antigens expressed on cells under stress conditions.

Expression of NKG2D ligands is regulated at different stages (transcription, mRNA and protein stabilization, cleavage from the cell surface) by various stress pathways. Among them, one of the most prominent stress pathways is DNA damage response. Genotoxic stress, stalled DNA replication, poorly regulated cell proliferation in tumorigenesis, viral replication or some viral products activate the ATM and ATR kinases. These kinases initiate the DNA damage response pathway which participates in NKG2D ligand upregulation. DNA damage response thus participate in alerting the immune system to the presence of potentially dangerous cells.

All NKG2D ligands are homologous to MHC class I molecules and are divided into two families: MIC and RAET1/ULBP. Human MIC genes are located within the MHC locus and are composed of seven members (MICA-G), of which only MICA and MICB produce functional transcripts. Among ten known human RAET1/ULBP genes, six encode functional proteins: RAET1E/ULBP4, RAET1G/ULBP5, RAET1H/ULBP2, RAET1/ULBP1, RAET1L/ULBP6, RAET1N/ULBP3.

In NK cells, NKG2D serves as an activating receptor, which itself is able to trigger cytotoxicity. NKG2D ligands are overexpressed by immunosuppressive cells of the TME, including myeloid-derived suppressor cells (MDSCs), M2-TAMS, and regulatory T cells (Tregs). In order to target these cells of the TME, a chimeric NKG2D receptor (NKG2D.ζ) has been overexpressed on human NK cells that enhanced NK cell activation, cytotoxicity, and cytokine secretion against NKG2D ligand-expressing tumors and MDSCs in TME models of human neuroblastoma [21]. In addition, recent studies have demonstrated that NKG2D ligands are selectively overexpressed on several pediatric cancers, including several sarcomas [22-24], thus allowing for targeting of both the tumor and TME.

In some embodiments of the disclosure, there are compositions comprising CARs which encompass a nucleotide and/or amino acid sequence of the NKG2D receptor or fragments thereof. As a reference sequence, an NKG2D polypeptide sequence is in the UniProtKB database at Accession Number P26718:

P26718
(SEQ ID NO: 22)
MGWIRGRRSRHSWEMSEFHNYNLDLKKSDFSTRWQKQRCPVVKS
KCRENASPFFFCCFIAVAMGIRFIIMVAIWSAVELNSLFNQEVQ
IPLTESYCGPCPKNWICYKNNCYQFFDESKNWYESQASCMSQNA
SLLKVYSKEDQDLLKLVKSYHWMGLVHIPINGSWQWEDGSILSP
NLLTIIEMQKGDCALYASSFKGYIENCSTPNTYICMQRTV

### C. Extracellular Spacer

An extracellular spacer may link the antigen-binding domain to the transmembrane domain. It should be flexible enough to allow the antigen-binding domain to orient in different directions to facilitate antigen binding. In one embodiment, the spacer is the hinge region from IgG. In some embodiments, the spacer is an IgG4 hinge. In some embodiments, the spacer is an IgG1 spacer. In some embodiments, the spacer comprises both an IgG4 hinge and an IgG1 spacer.

As used herein, the term “hinge” refers to a flexible polypeptide connector region (also referred to herein as “hinge region” or “spacer”) providing structural flexibility and spacing to flanking polypeptide regions and can consist of natural or synthetic polypeptides. A “spacer” derived from an immunoglobulin (e.g., IgG1) is generally defined as stretching from Glu216 to Pro230 of human IgG1, for example (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425. The hinge region can include a complete hinge region derived from an antibody of a different class or subclass from that of the CH1 domain. The term “hinge” can also include regions derived from CD8 and other receptors that provide a similar function in providing flexibility and spacing to flanking regions. Other alternatives include the CH2CH3 region of immunoglobulin and portions of CD3.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some embodiments, the extracellular spacer consists of or comprises a hinge region from an immunoglobulin (e.g. IgG). Immunoglobulin hinge region amino acid sequences are known in the art; see, e.g., Tan et al. (1990) Proc. Natl. Acad. Sci. USA 87: 162; and Huck et al. (1986) Nucl. Acids Res.

The length of an extracellular spacer may have effects on the CAR's signaling activity and/or the CAR-T cells' expansion properties in response to antigen-stimulated CAR signaling. In some embodiments, a shorter spacer such as less than 50, 45, 40, 30, 35, 30, 25, 20, 15, 14, 13, 12, 11, or 10 amino acids is used. In some embodiments, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

In some embodiments, the hinge region comprises SEQ ID NO:5. As non-limiting examples of additional hinge regions, an immunoglobulin hinge region can also include one of the following amino acid sequences: EPKSCDKTHTCPPCP (SEQ ID NO:23-human IgG1 hinge); ERKCCVECPPCP (SEQ ID NO:24-human IgG2 hinge); ELKTPLGDTTHTCPRCP (SEQ ID NO:25-human IgG3 hinge); or ESKYGPPCPSCP (SEQ ID NO:26) (human IgG4 hinges) and the like.

The extracellular spacer can comprise an amino acid sequence of a human IgG1, IgG2, IgG3, or IgG4 hinge region. The extracellular spacer may also include one or more amino acid substitutions and/or insertions and/or deletions compared to a wild-type (naturally-occurring) hinge region.

### D. Transmembrane Domain

With respect to the transmembrane domain, the CAR can be designed to comprise a transmembrane domain that is fused to the extracellular domain of the CAR. The transmembrane domain is a hydrophobic alpha helix that spans the membrane. Different transmembrane domains may result in different receptor stability. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

In various embodiments the transmembrane domain can be derived either from a natural or from a synthetic source. Where the source is natural, the domain may be derived from any membrane-bound or transmembrane protein. Illustrative, but non-limiting, examples of transmembrane regions of particular use in the CAR constructs contemplated here can be derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86, CD134, CD137, CD154. Alternatively, the transmembrane domain can be synthetic, in which case it can comprise predominantly hydrophobic residues such as leucine and valine. In certain embodiments, aa triplet of phenylalanine, tryptophan and valine will be found at each end of a synthetic transmembrane domain. Optionally, a short oligo- or polypeptide linker, e.g., between 2 and about 10 amino acids in length may form the linkage between the transmembrane domain and the cytoplasmic signaling domain of the CAR. In certain embodiments a glycine-serine doublet provides a particularly suitable linker.

The transmembrane domain is interposed between the extracellular spacer and the cytoplasmic region. In some embodiments, the transmembrane domain is interposed between the extracellular spacer and one or more costimulatory regions. In some embodiments, a linker is between the transmembrane domain and the one or more costimulatory regions.

Any transmembrane domain that provides for insertion of a polypeptide into the cell membrane of a eukaryotic (e.g., mammalian) cell may be suitable for use. As one non-limiting example, the transmembrane sequence FWVLVVVGGVLACYSLLVTVAFIIFWVRS (SEQ ID NO:7), which is CD28-derived, can be used. In some embodiments, the transmembrane domain is CD8 beta derived: LGLLVAGVLVLLVSLGVAIHLCC (SEQ ID NO:27); CD4 derived: ALIVLGGVAGLLLFIGLGIFFCVRC (SEQ ID NO:28); CD3 zeta derived: LCYLLDGILFIYGVILTALFLRV (SEQ ID NO:29); CD28 derived: WVLVVVGGVLACYSLLVTVAFIIFWV (SEQ ID NO:30); CD134 (OX40) derived: VAAILGLGLVLGLLGPLAILLALYLL (SEQ ID NO:31); or CD7 derived: ALPAALAVISFLLGLGLGVACVLA (SEQ ID NO:32). In some embodiments, the transmembrane domain is derived from CD28, CD8, CD4, CD3-zeta, CD134, or CD7.

### E. Cytoplasmic Region

The cytoplasmic domain or otherwise the intracellular signaling domain of the CAR is responsible for activation of at least one of the normal effector functions of the immune cell in which the CAR has been placed. After antigen and/or ligand recognition, receptors cluster and a signal is transmitted to the cell through the cytoplasmic region. In some embodiments, the costimulatory domains described herein are part of the cytoplasmic region.

The term “effector function” refers to a specialized function of a cell. An effector function of a T cell, for example, may be cytolytic activity, or helper activity including the secretion of cytokines. Thus the term “intracellular signaling domain” refers to the portion of a protein that transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

Cytoplasmic regions and/or costimulatiory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the cytoplasmic region includes at least one (e.g., one, two, three, four, five, six, etc.) ITAM motif as described herein. In some embodiments, the cytoplasmic region includes CD3-zeta, DAP10, CD28, 2B4, DNAM-1, 4-1BB, OX40, CD30, CD40, ICOS, lymphocyte function-associated antigen-1 (LFA-I), CD2, CD7, LIGHT, and NKG2C type signaling chains.

Cytoplasmic regions suitable for use in the polypeptides of the disclosure include immunoreceptor tyrosine-based activation motif (ITAM)-containing intracellular signaling polypeptides. An ITAM motif is YX₁X₂(L/I), where X₁ and X₂ are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX₁X₂(L/I))(X₃)ₙ(YX₁X₂(L/I)), where n is an integer from 6 to 8, and each of the 6-8 X₃ can be any amino acid.

A suitable cytoplasmic region may be an ITAM motif-containing a portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, FCER1G (Fc epsilon receptor I gamma chain); CD3E (CD3 epsilon); CD3G (CD3 gamma); CD3-zeta; and CD79A (antigen receptor complex-associated protein alpha chain).

In some cases, the cytoplasmic region is derived from DAP12 (also known as TYROBP; TYRO protein tyrosine kinase binding protein; KARAP; PLOSL; DN AX-activation protein 12; KAR-associated protein; TYRO protein tyrosine kinase-binding protein; killer activating receptor associated protein; killer-activating receptor-associated protein; etc.). For example, a suitable endodomain polypeptide can comprise an amino acid sequence having at least 75%, at least 80%, at least 85%, at least 90%, at least 95%, at least 98%, or 100%, amino acid sequence identity to:

(SEQ ID NO: 33)
MGGLEPCSRLLLLPLLLAVSGLRPVQAQAQSDCSCSTVSPGVLAG
IVMGDLVLTVLIALAVYFLGRLVPRGRGAAEAATRKQRITETESP
YQELQGQRSDVYSDLNTQRPYYK;
(SEQ ID NO: 34)
MGGLEPCSRLLLLPLLLAVSGLRPVQAQAQSDCSCSTVSPGVLAG
IVMGDLVLTVLIALAVYFLGRLVPRGRGAAEATRKQRITETESPY
QELQGQRSDVYSDLNTQRPYYK;
(SEQ ID NO: 35)
MGGLEPCSRLLLLPLLLAVSDCSCSTVSPGVLAGIVMGDLVLTVL
IALAVYFLGRLVPRGRGAAEAATRKQRITETESPYQELQGQRSDV
YSDLNTQRPYYK;
or
(SEQ ID NO: 36)
MGGLEPCSRLLLLPLLLAVSDCSCSTVSPGVLAGIVMGDLVLTVL
IALAVYFLGRLVPRGRGAAEATRKQRITETESPYQELQGQRSDVY
SDLNTQRPYYK

In some embodiments, a suitable cytoplasmic region can comprise an ITAM motif-containing a portion of the full length DAP12 amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 37)
ESPYQELQGQRSDVYSDLNTQ.

In some embodiments, the cytoplasmic region is derived from FCER1G (also known as FCRG; Fc epsilon receptor I gamma chain; Fc receptor gamma-chain; fc-epsilon R1-gamma; fcRgamma; fceRI gamma; high affinity immunoglobulin epsilon receptor subunit gamma; immunoglobulin E receptor, high affinity, gamma chain; etc.). For example, a suitable endodomain polypeptide can comprise an amino acid sequence having at least 75%, at least 80%, at least 85%, at least 90%, at least 95%, at least 98%, or 100% amino acid sequence identity to

(SEQ ID NO: 38)
MIPAVVLLLLLLVEQAAALGEPQLCYILDAILFLYGIVLTLLYCR
LKIQVRKAAITSYEKSDGVYTGLSTRNQETYETLKHEKPPQ.

In some embodiments, a suitable cytoplasmic region can comprise an ITAM motif-containing a portion of the full length FCER1G amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 39)
DGVYTGLSTRNQETYETLKHE.

In some embodiments, the cytoplasmic region is derived from T cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T cell receptor T3 delta chain; T cell surface glycoprotein CD3 delta chain; etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 170 aa, of either of the following amino acid sequences (2 isoforms):

(SEQ ID NO: 40)
MEHSTFLSGLVLATLLSQVSPFKIPIEELEDRVFVNCNTSITWVE
GTVGTLLSDITRLDLGKRILDPRGIYRCNGTDIYKDKESTVQVHY
RMCQSCVELDPATVAGIIVTDVIATLLLALGVFCFAGHETGRLSG
AADTQALLRNDQVYQPLRDRDDAQYSHLGGNWARNK
or
(SEQ ID NO: 41)
MEHSTFLSGLVLATLLSQVSPFKIPIEELEDRVFVNCNTSITWVE
GTVGTLLSDITRLDLGKRILDPRGIYRCNGTDIYKDKESTVQVHY
RTADTQALLRNDQVYQPLRDRDDAQYSHLGGNWARNK.

In some embodiments, a suitable cytoplasmic region can comprise an ITAM motif-containing a portion of the full length CD3 delta amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 42)
DQVYQPLRDRDDAQYSHLGGN.

In some embodiments, the cytoplasmic region is derived from T cell surface glycoprotein CD3 epsilon chain (also known as CD3e, T cell surface antigen T3/Leu-4 epsilon chain, T cell surface glycoprotein CD3 epsilon chain, AI504783, CD3, CD3epsilon, T3e, etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 205 aa, of the following amino acid sequence:

(SEQ ID NO: 43)
MQSGTHWRVLGLCLLSVGVWGQDGNEEMGGITQTPYKVSISGTTV
ILTCPQYPGSEILWQHNDKNIGGDEDDKNIGSDEDHLSLKEFSEL
EQSGYYVCYPRGSKPEDANFYLYLRARVCENCMEMDVMSVATIVI
VDICITGGLLLLVYYWSKNRKAKAKPVTRGAGAGGRQRGQNKERP
PPVPNPDYEPIRKGQRDLYSGLNQRRI

In some embodiments, a suitable cytoplasmic region can comprise an ITAM motif-containing a portion of the full length CD3 epsilon amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 44)
NPDYEPIRKGQRDLYSGLNQR.

In some embodiments, the cytoplasmic region is derived from T cell surface glycoprotein CD3 gamma chain (also known as CD3G, T cell receptor T3 gamma chain, CD3-GAMMA, T3G, gamma polypeptide (TiT3 complex), etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 180 aa, of the following amino acid sequence:

(SEQ ID NO: 45)
MEQGKGLAVLILAIILLQGTLAQSIKGNHLVKVYDYQEDGSVLLT
CDAEAKNITWFKDGKMIGFLTEDKKKWNLGSNAKDPRGMYQCKGS
QNKSKPLQVYYRMCQNCIELNAATISGFLFAEIVSIFVLAVGVYF
IAGQDGVRQSRASDKQTLLPNDQLYQPLKDREDDQYSHLQGNQLR
RN.

In some embodiments, a suitable cytoplasmic region can comprise an ITAM motif-containing a portion of the full length CD3 gamma amino acid sequence. Thus, a suitable cytoplasmic region can comprise an amino acid sequence having at least 75%, at least 80%, at least 85%, at least 90%, at least 95%, at least 98%, or 100%, amino acid sequence identity to

(SEQ ID NO: 46)
DQLYQPLKDREDDQYSHLQGN.

In some embodiments, the cytoplasmic region is derived from T cell surface glycoprotein CD3 zeta chain (also known as CD3Z, T cell receptor T3 zeta chain, CD247, CD3-ZETA, CD3H, CD3Q, T3Z, TCRZ, etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences (2 isoforms):

(SEQ ID NO: 47)
MKWKALFTAAILQAQLPITEAQSFGLLDPKLCYLLDGILFIYGVI
LTALFLRVKFSRSADAPAYQQGQNQLYNELNLGRREEYDVLDKRR
GRDPEMGGKPRRKNPQEGLYNELQKDKMAEAYSEIGMKGERRRGK
GHDGLYQGLSTATKDTYDALHMQALPPR
or
(SEQ ID NO: 48)
MKWKALFTAAILQAQLPITEAQSFGLLDPKLCYLLDGILFIYGVI
LTALFLRVKFSRSADAPAYQQGQNQLYNELNLGRREEYDVLDKRR
GRDPEMGGKPQRRKNPQEGLYNELQKDKMAEAYSEIGMKGERRRG
KGHDGLYQGLSTATKDTYDALHMQALPPR.

In some embodiments, the cytoplasmic region comprises the sequence:

(SEQ ID NO: 49)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR.

In some embodiments, the cytoplasmic region comprises the sequence:

(SEQ ID NO: 16)
MRVKFSRSAEPPAYQQGQNQLYNELNLGRREEYDVLDKRRGRDPEMGGK
PRRKNPQEGLYNELQKDKMAEAYSEIGMKGERRRGKGHDGLYQGLSTAT
KDTYDALHMQALPPR.

In some embodiments, a suitable cytoplasmic region can comprise an ITAM motif-containing a portion of the full length CD3 zeta amino acid sequence. Thus, a suitable cytoplasmic region can comprise an amino acid sequence having at least 75%, at least 80%, at least 85%, at least 90%, at least 95%, at least 98%, or 100%, amino acid sequence identity to any of the following amino acid sequences:

(SEQ ID NO: 50)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR;
(SEQ ID NO: 51)
NQLYNELNLGRREEYDVLDKR;
(SEQ ID NO: 52)
EGLYNELQKDKMAEAYSEIGMK;
or
(SEQ ID NO: 53)
DGLYQGLSTATKDTYDALHMQ.

In some embodiments, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 150 aa, from about 150 aa to about 200 aa, or from about 200 aa to about 220 aa, of either of the following amino acid sequences (2 isoforms):

(SEQ ID NO: 54)
MPGGPGVLQALPATIFLLFLLSAVYLGPGCQALWMHKVPASLMVSLGED
AHFQCPHNSSNNANVTWWRVLHGNYTWPPEFLGPGEDPNGTLIIQNVNK
SHGGIYVCRVQEGNESYQQSCGTYLRVRQPPPRPFLDMGEGTKNRIITA
EGIILLFCAVVPGTLLLFRKRWQNEKLGLDAGDEYEDENLYEGLNLDDC
SMYEDISRGLQGTYQDVGSLNIGDVQLEKP;
or
(SEQ ID NO: 55)
MPGGPGVLQALPATIFLLFLLSAVYLGPGCQALWMHKVPASLMVSLGED
AHFQCPHNSSNNANVTWWRVLHGNYTWPPEFLGPGEDPNEPPPRPFLDM
GEGTKNRIITAEGIILLFCAVVPGTLLLFRKRWQNEKLGLDAGDEYEDE
NLYEGLNLDDCSMYEDISRGLQGTYQDVGSLNIGDVQLEKP.

In some embodiments, a suitable cytoplasmic region can comprise an ITAM motif-containing a portion of the full length CD79A amino acid sequence. Thus, a suitable cytoplasmic region can comprise an amino acid sequence having at least 75%, at least 80%, at least 85%, at least 90%, at least 95%, at least 98%, or 100%, amino acid sequence identity to the following amino acid sequence:

(SEQ ID NO: 56)
ENLYEGLNLDDCSMYEDISRG.

In some embodiments, suitable cytoplasmic regions can comprise a CD28 type signaling chain. An example of a CD28 signaling chain is the amino acid sequence FWVLVVVGGVLACYSLLVTVAFIIFWVRSKRSRLLHSDYMNMTPRRPGPTRKHYQPYA PPRDFAAYRS (SEQ ID NO:57). In some embodiments, a suitable endodomain comprises an amino acid sequence having at least 85%, at least 90%, at least 95%, at least 98%, or at least 99%, amino acid sequence identity to the entire length of the amino acid sequence

(SEQ ID NO: 58)
FWVLVVVGGVLACYSLLVTVAFIIFWVRSKRSRLLHSDYMNMTPRRPGP
TRKHYQPYAPPRDFAAYRS.

Further cytoplasmic regions suitable for use in the polypeptides of the disclosure include a ZAP70 polypeptide, e.g., a polypeptide comprising an amino acid sequence having at least 85%, at least 90%, at least 95%, at least 98%, at least 99%, or 100%, amino acid sequence identity to a contiguous stretch of from about 300 amino acids to about 400 amino acids, from about 400 amino acids to about 500 amino acids, or from about 500 amino acids to 619 amino acids, of the following amino acid sequence:

(SEQ ID NO: 59)
MPDPAAHLPFFYGSISRAEAEEHLKLAGMADGLFLLRQCLRSLGGYVLS
LVHDVRFHHFPIERQLNGTYAIAGGKAHCGPAELCEFYSRDPDGLPCNL
RKPCNRPSGLEPQPGVFDCLRDAMVRDYVRQTWKLEGEALEQAIISQAP
QVEKLIATTAHERMPWYHSSLTREEAERKLYSGAQTDGKFLLRPRKEQG
TYALSLIYGKTVYHYLISQDKAGKYCIPEGTKFDTLWQLVEYLKLKADG
LIYCLKEACPNSSASNASGAAAPTLPAHPSTLTHPQRRIDTLNSDGYTP
EPARITSPDKPRPMPMDTSVYESPYSDPEELKDKKLFLKRDNLLIADIE
LGCGNFGSVRQGVYRMRKKQIDVAIKVLKQGTEKADTEEMMREAQIMHQ
LDNPYIVRLIGVCQAEALMLVMEMAGGGPLHKFLVGKREEIPVSNVAEL
LHQVSMGMKYLEEKNFVHRDLAARNVLLVNRHYAKISDFGLSKALGADD
SYYTARSAGKWPLKWYAPECINFRKFSSRSDVWSYGVTMWEALSYGQKP
YKKMKGPEVMAFIEQGKRMECPPECPPELYALMSDCWIYKWEDRPDFLT
VEQRMRACYYSLASKVEGPPGSTQKAEAACA.

### 1. Costimulatory Region

The term “costimulatory ligand,” as the term is used herein, includes a molecule on an antigen presenting cell (e.g., an APC, dendritic cell, B cell, and the like) that specifically binds a cognate costimulatory molecule on an immune effector cell, thereby providing a signal which, in addition to the primary signal to mediate the immune effector cell response, including, but not limited to, proliferation, activation, differentiation, and the like. A costimulatory ligand also encompasses, inter alia, an antibody that specifically binds with a costimulatory molecule present on an immune effector cell. A “costimulatory molecule” refers to the cognate binding partner on an immune effector cell that specifically binds with a costimulatory ligand, thereby mediating a co-stimulatory response by the immune effector, such as, but not limited to, proliferation and/or activation. A “costimulatory signal”, as used herein, refers to a signal, that in combination with a primary signal, leads to immune cell activation, proliferation, and/or upregulation or downregulation of key molecules.

By the term “stimulation,” it is meant a primary response induced by binding of a stimulatory molecule with its cognate ligand, thereby mediating a signal transduction event, such as, but not limited to, signal transduction. Stimulation can mediate altered expression of certain molecules. A “stimulatory molecule,” as the term is used herein, means a molecule on an immune effector cell that specifically binds with a cognate stimulatory ligand present on an antigen presenting cell. A “stimulatory ligand,” as used herein, means a ligand that when present on an antigen presenting cell (e.g., an APC, a dendritic cell, a B-cell, and the like) can specifically bind with a cognate binding partner (referred to herein as a “stimulatory molecule”) on an immune effector cell, thereby mediating a primary response by the immune effector cell, including, but not limited to, activation, initiation of an immune response, proliferation, and the like.

Non-limiting examples of suitable costimulatory regions, such as those included in the cytoplasmic region, include, but are not limited to, polypeptides from 4-1BB (CD137), CD28, ICOS, OX-40, BTLA, CD27, CD30, CD40, GITR, 2B4, DNAM-1, lymphocyte function-associated antigen-1 (LFA-1), CD2, CD7, LIGHT, NKG2C, and HVEM.

A co-stimulatory region may have a length of at least, at most, or exactly 10, 15, 20, 25, 30, 35, 40, 50, 60, 70, 80, 90, 100, 150, 200, or 300 amino acids or any range derivable therein.

In some embodiments, the costimulatory region is derived from 2B4 (also known as CD244, NAIL, NKR2B4, Nmrk, SLAMF4, CD244 molecule, etc.). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 8)
WRRKRKEKQSETSPKEFLTIYEDVKDLKTRRNHEQEQTFPGGGSTIYSM
IQSQSSAPTSQEPAYTLYSLIQPSRKSGSRKRNHSPSFNSTIYEVIGKS
QPKAQNPARLSRKELENFDVYS;
or
(SEQ ID NO: 60)
MLGQVVTLILLLLLKVYQGKGCQGSADHVVSISGVPLQLQPNSIQTKVD
SIAWKKLLPSQNGFHHILKWENGSLPSNTSNDRFSFIVKNLSLLIKAAQ
QQDSGLYCLEVTSISGKVQTATFQVFVFESLLPDKVEKPRLQGQGKILD
RGRCQVALSCLVSRDGNVSYAWYRGSKLIQTAGNLTYLDEEVDINGTHT
YTCNVSNPVSWESHTLNLTQDCQNAHQEFRFWPFLVIIVILSALFLGTL
ACFCVWRRKRKEKQSETSPKEFLTIYEDVKDLKTRRNHEQEQTFPGGGS
TIYSMIQSQSSAPTSQEPAYTLYSLIQPSRKSGSRKRNHSPSFNSTIYE
VIGKSQPKAQNPARLSRKELENFDVYS.

In some embodiments, the costimulatory region is derived from an intracellular portion of the transmembrane protein 4-1BB (also known as 41BB, 41BB-L, Tumor necrosis factor receptor superfamily member 9, TNFRSF9; CD137; CDw137; ILA; etc.). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 9)
KRGRKKLLYIFKQPFMRPVQTTQEEDGCSCRFPEEEEGGCEL;
or
(SEQ ID NO: 61)
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTNCSLDGKSVL
VNGTKERDVVCGPSPADLSPGASSVTPPAPAREPGHSPQIISFFLALTS
TALLFLLFFLTLRFSVVKRGRKKLLYIFKQPFMRPVQTTQEEDGCSCRF
PEEEEGGCEL.

In some embodiments, the costimulatory region derived from DNAM-1 (also known as CD226, DNAM1, PTA1, TLiSA1, CD226 molecule, etc.). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 10)
NRRRRRERRDLFTESWDTQKAPNNYRSPISTSQPTNQSMDDTREDIYVN
YPTFSRRPKTRV;
or
(SEQ ID NO: 62)
MDYPTLLLALLHVYRALCEEVLWHTSVPFAENMSLECVYPSMGILTQVE
WFKIGTQQDSIAIFSPTHGMVIRKPYAERVYFLNSTMASNNMTLFFRNA
SEDDVGYYSCSLYTYPQGTWQKVIQVVQSDSFEAAVPSNSHIVSEPGKN
VTLTCQPQMTWPVQAVRWEKIQPRQIDLLTYCNLVHGRNFTSKFPRQIV
SNCSHGRWSVIVIPDVTVSDSGLYRCYLQASAGENETFVMRLTVAEGKT
DNQYTLFVAGGTVLLLLFVISITTIIVIFLNRRRRRERRDLFTESWDTQ
KAPNNYRSPISTSQPTNQSMDDTREDIYVNYPTFSRRPKTRV.

In some embodiments, the costimulatory region is derived from an intracellular portion of the transmembrane protein OX-40 (also known as tumor necrosis factor receptor superfamily member 4, TNFRSF4, RP5-902P8.3, ACT35, CD134, OX40, TXGP1L). For example, a suitable co-stimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 11)
RDQRLPPDAHKPPGGGSFRTPIQEEQADAHSTLAKI;
or
(SEQ ID NO: 63)
MVSRCSRSQNTVCRPCGPGFYNDVVSSKPCKPCTWCNLRSGSERKQLCT
ATQDTVCRCRAGTQPLDSYKPGVDCAPCPPGHFSPGDNQACKPWTNCTL
AGKHTLQPASNSSDAICEDRDPPATQPQETQGPPARPITVQPTEAWPRT
SQGPSTRPVEVPGGRAVAAILGLGLVLGLLGPLAILLALYLLRRDQRLP
PDAHKPPGGGSFRTPIQEEQADAHSTLAKI.

In some embodiments, the costimulatory region is derived from DAP10 (also known as HCST, KAP10, PIK3AP, hematopoietic cell signal transducer; etc.). For example, a suitable costimulatory region can comprise an amino acid sequence having at least 75%, at least 80%, at least 85%, at least 90%, at least 95%, at least 98%, or 100%, amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 64)
MIHLGHILFLLLLPVAAAQTTPGERSSLPAFYPGTSGSCSGCGSLSLPL
LAGLVAADAVASLLIVGAVFLCARPRRSPAQEDGKVYINMPGRG;
or
(SEQ ID NO: 65)
MIHLGHILFLLLLPVAAAQTTPGERSSLPAFYPGTSGSCSGCGSLSLPL
LAGLVAADAVASLLIVGAVFLCARPRRSPAQDGKVYINMPGRG

In some embodiments, the costimulatory region is derived from an intracellular portion of the transmembrane protein CD28 (also known as Tp44). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 66)
MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFY
LQNLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS.

In some embodiments, the costimulatory region is derived from an intracellular portion of the transmembrane protein ICOS (also known as inducible T-cell costimulatory, AILIM, CD278, and CVID1). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 67)
MKSGLWYFFLFCLRIKVLTGEINGSANYEMFIFHNGGVQILCKYPDIVQ
QFKMQLLKGGQILCDLTKTKGSGNTVSIKSLKFCHSQLSNNSVSFFLYN
LDHSHANYYFCNLSIFDPPPFKVTLTGGYLHIYESQLCCQLKFWLPIGC
AAFVVVCILGCILICWLTKKKYSSSVHDPNGEYMFMRAVNTAKKSRLTD
VTL.

In some embodiments, the costimulatory region is derived from an intracellular portion of the transmembrane protein BTLA (also known as B- and T-Lymphocyte-Associated Protein, BTLA1 and CD272). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 68)
MKTLPAMLGTGKLFWVFFLIPYLDIWNIHGKESCDVQLYIKRQSEHSIL
AGDPFELECPVKYCANRPHVTWCKLNGTTCVKLEDRQTSWKEEKNISFF
ILHFEPVLPNDNGSYRCSANFQSNLIESHSTTLYVTDVKSASERPSKDE
MASRPWLLYRLLPLGGLPLLITTCFCLFCCLRRHQGKQNELSDTAGREI
NLVDAHLKSEQTEASTRQNSQVLLSETGIYDNDPDLCFRMQEGSEVYSN
PCLEENKPGIVYASLNHSVIGPNSRLARNVKEAPTEYASICVRS.

In some embodiments, the costimulatory region is derived from an intracellular portion of the transmembrane protein CD27 (also known as S152, T14, Tumor Necrosis Factor Receptor Superfamily Member 7, TNFRSF7, and Tp55). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 69)
MARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTI
TANAECACRNGWQCRDKECTECDPLPNPSLTARSSQALSPHPQPTHLPY
VSEMLEARTAGHMQTLADFRQLPARTLSTHWPPQRSLCSSDFIRILVIF
SGMFLVFTLAGALFLHQRRKYRSNKGESPVEPAEPCHYSCPREEEGSTI
PIQEDYRKPEPACSP.

In some embodiments, the costimulatory region is derived from an intracellular portion of the transmembrane protein CD30 (also known as tumor necrosis factor receptor superfamily member 8, TNFRSF8, D1S166E, and Ki-1). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 70)
MRVLLAALGLLFLGALRAFPQDRPFEDTCHGNPSHYYDKAVRRCCYRCPMG
LFPTQQCPQRPTDCRKQCEPDYYLDEADRCTACVTCSRDDLVEKTPCAWNS
SRVCECRPGMFCSTSAVNSCARCFFHSVCPAGMIVKFPGTAQKNTVCEPAS
PGVSPACASPENCKEPSSGTIPQAKPTPVSPATSSASTMPVRGGTRLAQEA
ASKLTRAPDSPSSVGRPSSDPGLSPTQPCPEGSGDCRKQCEPDYYLDEAGR
CTACVSCSRDDLVEKTPCAWNSSRTCECRPGMICATSATNSCARCVPYPIC
AAETVTKPQDMAEKDTTFEAPPLGTQPDCNPTPENGEAPASTSPTQSLLVD
SQASKTLPIPTSAPVALSSTGKPVLDAGPVLFWVILVLVVVVGSSAFLLCH
RRACRKRIRQKLHLCYPVQTSQPKLELVDSRPRRSSTQLRSGASVTEPVAE
ERGLMSQPLMETCHSVGAAYLESLPLQDASPAGGPSSPRDLPEPRVSTEHT
NNKIEKIYIMKADTVIVGTVKAELPEGRGLAGPAEPELEEELEADHTPHYP
EQETEPPLGSCSDVMLSVEEEGKEDPLPTAASGK.

In some embodiments, the costimulatory region is derived from an intracellular portion of the transmembrane protein GITR (also known as tumor necrosis factor receptor superfamily member 18, TNFRSF18, RP5-902P8.2, AITR, CD357, ENERGEN, and GITR-D). For example, a suitable co-stimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 71)
MAQHGAMGAFRALCGLALLCALSLGQRPTGGPGCGPGRLLLGTGTDARCCR
VHTTRCCRDYPGEECCSEWDCMCVQPEFHCGDPCCTTCRHHPCPPGQGVQS
QGKFSFGFQCIDCASGTFSGGHEGHCKPWTDCTQFGFLTVFPGNKTHNAVC
VPGSPPAEPLGWLTVVLLAVAACVLLLTSAQLGLHIWQLRSQCMWPRETQL
LLEVPPSTEDARSCQFPEEERGERSAEEKGRLGDLWV.

In some embodiments, the costimulatory region derived from an intracellular portion of the transmembrane protein HVEM (also known as tumor necrosis factor receptor superfamily member 14, TNFRSF14, RP3-395M20.6, ATAR, CD270, HVEA, LIGHTR, and TR2). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 72)
MEPPGDWGPPPWRSTPKTDVLRLVLYLTFLGAPCYAPALPSCKEDEYPVGS
ECCPKCSPGYRVKEACGELTGTVCEPCPPGTYIAHLNGLSKCLQCQMCDPA
MGLRASRNCSRTENAVCGCSPGHFCIVQDGDHCAACRAYATSSPGQRVQKG
GTESQDTLCQNCPPGTFSPNGTLEECQHQTKCSWLVTKAGAGTSSSHWVWW
FLSGSLVIVIVCSTVGLIICVKRRKPRGDVVKVIVSVQRKRQEAEGEATVI
EALQAPPDVTTVAVEETIPSFTGRSPNH.

In some embodiments, the costimulatory region derived from CD40 (also known as Bp50, CDW40, TNFRSF5, p50, CD40 (protein), CD40 molecule, etc.). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 73)
MVRLPLQCVLWGCLLTAVHPEPPTACREKQYLINSQCCSLCQPGQKLVSDC
TEFTETECLPCGESEFLDTWNRETHCHQHKYCDPNLGLRVQQKGTSETDTI
CTCEEGWHCTSEACESCVLHRSCSPGFGVKQIATGVSDTICEPCPVGFFSN
VSSAFEKCHPWTSCETKDLVVQQAGTNKTDVVCGPQDRLRALVVIPIIFGI
LFAILLVLVFIKKVAKKPTNKAPHPKQEPQEINFPDDLPGSNTAAPVQETL
HGCQPVTQEDGKESRISVQERQ.

In some embodiments, the costimulatory region derived from LFA-1 (also known as lymphocyte function-associated antigen 1, integrin alpha L, ITGAL, CD11A, LFA1A, integrin subunit alpha L, etc.). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 74)
MKDSCITVMAMALLSGFFFFAPASSYNLDVRGARSFSPPRAGRHFGYRVLQV
GNGVIVGAPGEGNSTGSLYQCQSGTGHCLPVTLRGSNYTSKYLGMTLATDPTDGSILAC
DPGLSRTCDQNTYLSGLCYLFRQNLQGPMLQGRPGFQECIKGNVDLVFLFDGSMSLQPD
EFQKILDFMKDVMKKLSNTSYQFAAVQFSTSYKTEFDFSDYVKRKDPDALLKHVKHML
LLTNTFGAINYVATEVFREELGARPDATKVLIIITDGEATDSGNIDAAKDIIRYIIGIGKHF
QTKESQETLHKFASKPASEFVKILDTFEKLKDLFTELQKKIYVIEGTSKQDLTSFNMELSS
SGISADLSRGHAVVGAVGAKDWAGGFLDLKADLQDDTFIGNEPLTPEVRAGYLGYTVT
WLPSRQKTSLLASGAPRYQHMGRVLLFQEPQGGGHWSQVQTIHGTQIGSYFGGELCGV
DVDQDGETELLLIGAPLFYGEQRGGRVFIYQRRQLGFEEVSELQGDPGYPLGRFGEAITA
LTDINGDGLVDVAVGAPLEEQGAVYIFNGRHGGLSPQPSQRIEGTQVLSGIQWFGRSIHG
VKDLEGDGLADVAVGAESQMIVLSSRPVVDMVTLMSFSPAEIPVHEVECSYSTSNKMK
EGVNITICFQIKSLIPQFQGRLVANLTYTLQLDGHRTRRRGLFPGGRHELRRNIAVTTSMS
CTDFSFHFPVCVQDLISPINVSLNFSLWEEEGTPRDQRAQGKDIPPILRPSLHSETWEIPFE
KNCGEDKKCEANLRVSFSPARSRALRLTAFASLSVELSLSNLEEDAYWVQLDLHFPPGL
SFRKVEMLKPHSQIPVSCEELPEESRLLSRALSCNVSSPIFKAGHSVALQMMENTLVNSS
WGDSVELHANVTCNNEDSDLLEDNSATTIIPILYPINILIQDQEDSTLYVSFTPKGPKIHQV
KHMYQVRIQPSIHDHNIPTLEAVVGVPQPPSEGPITHQWSVQMEPPVPCHYEDLERLPDA
AEPCLPGALFRCPVVFRQEILVQVIGTLELVGEIEASSMFSLCSSLSISFNSSKHFHLYGSN
ASLAQVVMKVDVVYEKQMLYLYVLSGIGGLLLLLLIFIVLYKVGFFKRNLKEKMEAGR
GVPNGIPAEDSEQLASGQEAGDPGCLKPLHEKDSESGGGKD.

In some embodiments, the costimulatory region derived from CD2 (also known as Lymphocyte-Function Antigen-2, LFA-2, SRBC, T11, CD2 molecule, etc.). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 75)
MSFPCKFVASFLLIFNVSSKGAVSKEITNALETWGALGQDINLDIPSFQMS
DDIDDIKWEKTSDKKKIAQFRKEKETFKEKDTYKLFKNGTLKIKHLKTDDQ
DIYKVSIYDTKGKNVLEKIFDLKIQERVSKPKISWTCINTTLTCEVMNGTD
PELNLYQDGKHLKLSQRVITHKWTTSLSAKFKCTAGNKVSKESSVEPVSCP
EKGLDIYLIIGICGGGSLLMVFVALLVFYITKRKKQRSRRNDEELETRAHR
VATEERGRKPHQIPASTPQNPATSQHPPPPPGHRSQAPSHRPPPPGHRVQH
QPQKRPPAPSGTQVHQQKGPPLPRPRVQPKPPHGAAENSLSPSSN

In some embodiments, the costimulatory region derived from CD7 (also known as GP40, LEU-9, TP41, Tp40, CD7 molecule, etc.). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEO ID NO: 76)
MAGPPRLLLLPLLLALARGLPGALAAQEVQQSPHCTTVPVGASVNITCSTS
GGLRGIYLRQLGPQPQDIIYYEDGVVPTTDRRFRGRIDFSGSQDNLTITMH
RLQLSDTGTYTCQAITEVNVYGSGTLVLVTEEQSQGWHRCSDAPPRASALP
APPTGSALPDPQTASALPDPPAASALPAALAVISFLLGLGLGVACVLARTQ
IKKLCSWRDKNSAACVVYEDMSHSRCNTLSSPNQYQ.

In some embodiments, the costimulatory region derived from LIGHT (also known as TNFSF14, CD258, HVEML, LIGHT, LTg, TR2, TNLG1D, tumor necrosis factor superfamily member 14, etc.). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 77)
MEESVVRPSVFVVDGQTDIPFTRLGRSHRRQSCSVARVGLGLLLLLMGAGL
AVQGWFLLQLHWRLGEMVTRLPDGPAGSWEQLIQERRSHEVNPAAHLTGAN
SSLTGSGGPLLWETQLGLAFLRGLSYHDGALVVTKAGYYYIYSKVQLGGVG
CPLGLASTITHGLYKRTPRYPEELELLVSQQSPCGRATSSSRVWWDSSFLG
GVVHLEAGEKVVVRVLDERLVRLRDGTRSYFGAFMV.

In some embodiments, the costimulatory region derived from NKG2C (also known as KLRC2, CD159c, NKG2-C, killer cell lectin like receptor C2, etc.). For example, a suitable costimulatory region can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 78)
MSKQRGTFSEVSLAQDPKRQQRKPKGNKSSISGTEQEIFQVELNLQNPSLN
HQGIDKIYDCQGLLPPPEKLTAEVLGIICIVLMATVLKTIVLIPFLEQNNS
SPNTRTQKARHCGHCPEEWITYSNSCYYIGKERRTWEESLLACTSKNSSLL
SIDNEEEMKFLASILPSSWIGVFRNSSHHPWVTINGLAFKHKIKDSDNAEL
NCAVLQVNRLKSAQCGSSMIYHCKHKL.

### F. Detection Peptides

In some embodiments, the polypeptides described herein may further comprise a detection peptide or molecule. Suitable detection peptides include hemagglutinin (HA; e.g., YPYDVPDYA (SEQ ID NO:79); FLAG (e.g., DYKDDDDK (SEQ ID NO:80); c-myc (e.g., EQKLISEEDL; SEQ ID NO:81), and the like.

In some embodiments, the detection molecule comprises a polypeptide comprising an IRES sequence and truncated CD19 (IRES-tCD19) sequence comprising the transmembrane and extracellular domains of CD19 downstream of an IRES sequence. For example, a suitable IRES sequence can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 18)
YVNSAPLPPPPLTLLAEAAWNKAGVRLSICYFPPYCRLLAMGPGNLALSSR
AFLGVFPLSPKECKVCMSRKQFLWKLLEDKQRLRPFAGSGTPHLATGASAA
KSHVYKIHLQRRHNPSATLVGLWKESNGSPQAYSTRGRMPRRYPIVWDLIW
GLGAHALHVFSRGKTSRPPEPRGRGFPLKNTII.

A suitable tCD19 sequence can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 19)
MPPPRLLFFLLFLTPMEVRPEEPLVVKVEEGDNAVLQCLKGTSDGPTQQLT
WSRESPLKPFLKLSLGLPGLGIHMRPLAIWLFIFNVSQQMGGFYLCQPGPP
SEKAWQPGWTVNVEGSGELFRWNVSDLGGLGCGLKNRSSEGPSSPSGKLMS
PKLYVWAKDRPEIWEGEPPCLPPRDSLNQSLSQDLTMAPGSTLWLSCGVPP
DSVSRGPLSWTHVHPKGPKSLLSLELKDDRPARDMWVMETGLLLPRATAQD
AGKYYCHRGNLTMSFHLEITARPVLWHWLLRTGGWKVSAVTLAYLIFCLCS
LVGILHLQRALVLRRKRKRMTDPTRRF.

In some embodiments, the detection molecule comprises a polypeptide comprising an IRES sequence and an NGFR sequence (also known as CD271, Gp80-LTNFRSF16, p75(NTR), p75NTR, nerve growth factor receptor, etc.). For example, a suitable NGFR sequence can comprise an amino acid sequence having at least 75%, at least 80%, at least 85%, at least 90%, at least 95%, at least 98%, or 100% amino acid sequence identity to and/or a length of at least, at most, or exactly 10, 15, 20, 25, 30, 35, 40, 50, 60, 70, 80, 90, 100, 150, 200, or 300 amino acids or any range derivable therein of an amino acid sequence comprising:

(SEQ ID NO: 82)
MGAGATGRAMDGPRLLLLLLLGVSLGGAKEACPTGLYTHSGECCKACNLGE
GVAQPCGANQTVCEPCLDSVTFSDVVSATEPCKPCTECVGLQSMSAPCVEA
DDAVCRCAYGYYQDETTGRCEACRVCEAGSGLVFSCQDKQNTVCEECPDGT
YSDEANHVDPCLPCTVCEDTERQLRECTRWADAECEEIPGRWITRSTPPEG
SDSTAPSTQEPEAPPEQDLIASTVAGVVTTVMGSSQPVVTRGTTDNLIPVY
CSILAAVVVGLVAYIAFKRWNS.

Other suitable detection peptides are known in the art.

### G. Therapeutic Controls

In some embodiments of the methods and compositions described herein, the CAR molecule is co-expressed with a therapeutic control.

Therapeutic controls regulate cell proliferation, facilitate cell selection (for example selecting cells which express the chimeric antigen receptors of the disclosure) or a combination thereof. In one embodiment, regulating cell proliferation comprises up-regulating cell proliferation to promote cell propagation. In another embodiment, regulating cell proliferation comprises down-regulating cell proliferation so as to reduce or inhibit cell propagation. In some embodiments, the agents that serve as therapeutic controls may promote enrichment of cells which express the chimeric antigen receptors which may result in a therapeutic advantage. In some embodiments, agents which serve as therapeutic controls may biochemically interact with additional compositions so as to regulate the functioning of the therapeutic controls. For example, EGFRt (a therapeutic control) may biochemically interact with cetuximab so as to regulate the function of EGFRt in selection, tracking, cell ablation or a combination thereof.

Exemplary therapeutic controls include truncated epidermal growth factor receptor (EGFRt), chimeric cytokine receptors (CCR) and/or dihydroxyfolate receptor (DHFR) (e.g., mutant DHFR). The polynucleotides encoding the CAR and the therapeutic control(s) may be linked via IRES sequences or via polynucleotide sequences encoding cleavable linkers. The CARs of the disclosure are constructed so that they may be expressed in cells, which in turn proliferate in response to the presence of at least one molecule that interacts with at least one antigen-specific targeting region, for instance, an antigen. In further embodiments, the therapeutic control comprises a cell-surface protein wherein the protein lacks intracellular signaling domains. It is contemplated that any cell surface protein lacking intracellular signaling or modified (e.g. by truncation) to lack intracellular signaling may be used. Further examples of a therapeutic control include truncated LNGFR, truncated CD19 etc. . . . wherein the truncated proteins lack intracellular signaling domains.

“Co-express” as used herein refers to simultaneous expression of two or more genes. Genes may be nucleic acids encoding, for example, a single protein or a chimeric protein as a single polypeptide chain. For example, the CARs of the disclosure may be co-expressed with a therapeutic control (for example truncated epidermal growth factor (EGFRt)), wherein the CAR is encoded by a first polynucleotide chain and the therapeutic control is encoded by a second polynucleotide chain. In an embodiment, the first and second polynucleotide chains are linked by a nucleic acid sequence that encodes a cleavable linker The polynucleotides encoding the CAR and the therapeutic control system may be linked by IRES sequences. Alternately, the CAR and the therapeutic control are encoded by two different polynucleotides that are not linked via a linker but are instead encoded by, for example, two different vectors. Further, the CARs of the disclosure may be co-expressed with a therapeutic control and CCR, a therapeutic control and DHFR (for example mutant DHFR) or a therapeutic control and CCR and DHFR (for example mutant DHFR). The CAR, therapeutic control and CCR may be co-expressed and encoded by first, second and third polynucleotide sequences, respectively, wherein the first, second and third polynucleotide sequences are linked via IRES sequences or sequences encoding cleavable linkers. Alternately, these sequences are not linked via linkers but instead are encoded via, for example, separate vectors. The CAR, therapeutic control and DHFR (for example mutant DHFR) may be co-expressed and encoded by first, second and fourth polynucleotide sequences, respectively, wherein the first, second and fourth polynucleotide sequences are linked via IRES sequences or via sequences encoding cleavable linkers. Alternately, these sequences are not linked via linkers but instead encoded via, for example, separate vectors. The CAR, therapeutic control, CCR and DHFR (for example mutant DHFR) may be co-expressed and encoded by first, second, third and fourth polynucleotide sequences, respectively, wherein the first, second, third and fourth polynucleotide sequences are linked via IRES sequences or sequences encoding cleavable linkers. Alternately, these sequences are not linked via linkers but instead are encoded via, for example, separate vectors. If the aforementioned sequences are encoded by separate vectors, these vectors may be simultaneously or sequentially transfected.

Further aspects of the therapeutic controls, CAR molecules, and methods of use for the compositions of the disclosure can be found in U.S. Pat. No. 9,447,194, which is herein incorporated by reference for all purposes.

### H. Additional Modifications and Polypeptide Embodiments

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs: 1-82.

It is contemplated that a region or fragment of a polypeptide of the disclosure may have an amino acid sequence that has, has at least or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, or contiguous amino acid deletions with respect to any of SEQ ID NOs: 1-82. Alternatively, a region or fragment of a polypeptide of the disclosure may have an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOs:1-82. Moreover, in some embodiments, a region or fragment comprises an amino acid region of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 in any of SEQ ID NOs: 1-82 (where position 1 is at the N-terminus of the SEQ ID NOs:1-82). The polypeptides of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%. 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%. 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000, 1500, or 2000 or more contiguous amino acids or nucleic acids, or any range derivable therein, of any of SEQ ID NOs: 1-82.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 650, 700, 750, 800, 850, 900, 1000, 1500, or 2000 of any of SEQ ID NOs: 1-82 (or any derivable range therein).

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

Additionally, the polypeptides of the disclosure may be chemically modified. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa. Non-conservative substitutions may involve the exchange of a member of one of the amino acid classes for a member from another class.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the disclosure, those that are within ±1 are included, and in other aspects of the disclosure, those within ±0.5 are included.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within +2 are included, in other embodiments, those which are within +1 are included, and in still other embodiments, those within +0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. Sec, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### V. Formulations and Culture of Cells

In particular embodiments, the immune effector cells and/or precursors thereto may be specifically formulated and/or they may be cultured in a particular medium at any stage of a process of generating the immune effector cells which express one or more of the CARs disclosed herein. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In further embodiments, the medium may be a serum-free medium that is suitable for cell development. For example, the medium may comprise B-27® supplement, xeno-free B-27® supplement (available at world wide web at thermofisher.com/us/en/home/technical-resources/media-formulation.250.html), NS21 supplement (Chen et al., J Neurosci Methods, 2008 Jun. 30; 171(2): 239-247, incorporated herein in its entirety), GS21™ supplement (available at world wide web at amsbio.com/B-27.aspx), or a combination thereof at a concentration effective for producing T cells from the 3D cell aggregate.

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine).

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21™ supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese.

In further embodiments, the medium may comprise externally added ascorbic acid. The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

The medium used may be supplemented with at least one externally added cytokine at a concentration from about 0.1 ng/ml to about 500 ng/ml, more particularly 1 ng/ml to 100 ng/ml, or at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein. Suitable cytokines, include but are not limited to, FLT3 ligand (FLT3L), interleukin 7 (IL-7), stem cell factor (SCF), thrombopoietin (TPO), IL-2, IL-4, IL-6, IL-15, IL-21, TNF-alpha, TGF-beta, interferon-gamma, interferon-lambda, TSLP, thymopentin, pleotrophin, and/or midkine. Particularly, the culture medium may include at least one of FLT3L and IL-7. More particularly, the culture may include both FLT3L and IL-7.

Other culturing conditions can be appropriately defined. For example, the culturing temperature can be about 20 to 40° C., such as at least, at most, or about 20, 21, 22, 23, 24, 25, 26, 27, 28, 29, 30, 31, 32, 33, 34, 35, 36, 37, 38, 39, 40° C. (or any range derivable therein), though the temperature may be above or below these values. The CO2 concentration can be about 1, 2, 3, 4, 5, 6, 7, 8, 9, or 10% (or any range derivable therein), such as about 2% to 10%, for example, about 2 to 5%, or any range derivable therein. The oxygen tension can be at least or about 1, 5, 8, 10, 20%, or any range derivable therein.

In specific embodiments, NK cells which express one or more of the CARs disclosed herein are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In some embodiments, the NK cell or population thereof comprise a one or more chimeric antigen receptors (CARs). Examples of tumor cell antigens to which a CAR may be directed include at least 5T4, 8H9, αᵥβ₆ integrin, BCMA, B7-H3, B7-H6, CAIX, CA9, CD19, CD20, CD22, CD30, CD33, CD38, CD44, CD44v6, CD44v7/8, CD70, CD123, CD138, CD171, CD133, CEA, c-Met, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FRα, GD2, G250/CAIX, GD3, Glypican-3 (GPC3), GUCY2C, HER1, HER2, ICAM-1, IL-13Rα2, IL-11Rα, Kras, Kras G12D, L1CAM, Lambda, Lewis-Y, Kappa, KDR, MAGE, MCSP, MET, Mesothelin, Muc1, Muc16, MUC18, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pme117, TRP-1/-2, P. polypeptide, MC1R, Prostate-specific antigen, β-catenin, BRCA1/2, CML66, Fibronectin, MART-2, TGF-βRII, WT-1, or VEGF receptors (e.g., VEGFR2), for example. The CAR may be a first, second, third, or more generation CAR. The CAR may be bispecific for any two nonidentical antigens, or it may be specific for more than two nonidentical antigens.

The therapeutic compositions and treatments disclosed herein may comprise administration of a combination of therapeutic agents, such as a first therapeutic or pharmaceutical composition or treatment and a second therapeutic or pharmaceutical composition or treatment. The therapies may be administered in any suitable manner known in the art. For example, the therapeutic or pharmaceutical compositions or treatments may be administered sequentially (at different times) or concurrently (at the same time). In some embodiments, the therapeutic or pharmaceutical compositions or treatments are administered in a separate composition. In some embodiments, the therapeutic or pharmaceutical compositions or treatments are in the same composition.

Embodiments of the disclosure relate to compositions and methods comprising therapeutic compositions. The different therapeutic or pharmaceutical compositions or treatments may be administered in one composition or in more than one composition, such as 2 compositions, 3 compositions, or 4 compositions. Various combinations of the agents may be employed.

The therapeutic or pharmaceutical compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic or pharmaceutical agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administerable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the therapeutic composition or treatment administered to a human will be in the range of about 10² up to about 10¹⁰ cells per kg of patient body weight whether by one or more administrations. In some embodiments, the therapy used is about 10² cells to about 10⁹ cells/kg, about 10² cells to about 10⁸ cells/kg, about 10² cells to about 10⁷ cells/kg, about 10² cells to about 10⁶ cells/kg, about 10² cells to about 10⁵ cells/kg, about 10² cells to about 10⁴ cells/kg, or about 10² cells to about 10³ cells/kg administered daily, for example. In one embodiment, a therapy described herein is administered to a subject at a dose of about 10² cells, about 10³ cells, about 10⁴ cells, about 10⁵ cells, about 10⁶ cells, about 10⁷ cells, about 10⁸ cells, about 10⁹ cells, or about 10¹⁰ cells. The dose may be administered as a single dose or as multiple doses (e.g., 2 or 3 doses), such as infusions. The progress of this therapy is easily monitored by conventional techniques.

In some embodiments, the therapeutically effective or sufficient amount of the therapeutic composition or treatment administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

In various embodiments the CAR-modified cells described herein can be administered cither as a therapeutic or pharmaceutical composition alone, or as a therapeutic or pharmaceutical composition in combination with diluents and/or with other components such other cytokines or cell populations. Briefly, in certain embodiments, pharmaceutical compositions can comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical or therapeutic compositions of the present disclosure may be administered in a manner appropriate to the disease to be treated (or prevented). The quantity and frequency of administration will be determined by such factors as the condition of the patient, and the type and severity of the subject's disease, although appropriate dosages may be determined by clinical trials. Precise amounts of the therapeutic or pharmaceutical composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

When “an immunologically effective amount”, “an anti-tumor effective amount”, “an tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject).

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VI. Methods of Producing Immune Effector Cell-Expressing CARs

Immune effector cells which express one or more of the CARs disclosed herein may be produced by any suitable method(s). The method(s) may utilize one or more successive steps for one or more modifications to cells and/or utilize one or more simultaneous steps for one or more modifications to cells.

Human NK cells were activated, transduced with retroviral constructs and expanded as previously described by the inventors' laboratory (Lapteva N, et al. Large-scale ex vivo expansion and characterization of natural killer cells for clinical applications. Cytotherapy. 2012; 14(9): 1131-43). Briefly, peripheral blood mononuclear cells (PBMCs) obtained from healthy donors were cocultured with irradiated (100 Gy) K562-mb15-41BB-L at a 1:10 (NK cell:irradiated tumor cell) ratio in G-Rex® cell culture devices (Wilson Wolf, St. Paul, MN, USA) for 4 days in Stem Cell Growth Medium (CellGenix) supplemented with 10% FBS and 500 IU/mL IL2. Cell suspensions on day 4 (containing 50-70% expanded/activated NK cells) were transduced with SFG-based retroviral vectors, as previously described (Xu Y, et al. Closely related T-memory stem cells correlate with in vivo expansion of CAR·CD19-T cells and are preserved by IL-7 and IL-15. Blood. 2014; 123(24):3750-9.). The transduced cell population was then subjected to secondary expansion to generate adequate cell numbers in G-Rex® devices at the same NK cell:irradiated tumor cell ratio with 100 IU/mL IL2. This 17-day human gene-modified NK cell protocol can result in >97% pure CD56⁺/CD3⁻ NK cell population with avg. 77.4%±18.2% (n=25) of NK cells transduced with a construct of interest. In some embodiments, transduced NK cells are purified to >95% by magnetic column selection of truncated CD19 or NGFR selection marker-positive cells.

Cells produced by the preparation methods may be frozen. The produced cells may be in a solution comprising dextrose, one or more electrolytes, albumin, dextran, and DMSO. The solution may be sterile, nonpyogenic, and isotonic.

Preparation methods of the disclosure may produce a population of immune effector cells comprising at least about 10²-10⁶ clonal cells. The method may produce a cell population comprising at least about 10⁶-10¹² total cells. The produced cell population may be frozen and then thawed. In some cases of the preparation method, the method further comprises introducing one or more additional nucleic acids into the frozen and thawed cell population, such as the one or more additional nucleic acids encoding one or more therapeutic gene products, for example.

Genetic modification may also be introduced to certain components to generate antigen- and/or ligand-specific immune effector cells and to model positive and negative selection. These modifications include, for example, transduction of immune effector cells with a vector encoding an antigen- and/or ligand-specific chimeric antigen receptor (CAR) for the generation of antigen- and/or ligand-specific cells and/or transduction of the immune effector cell lines with an antigen and/or ligand plus costimulatory molecules or cytokines to enhance the positive selection of CAR immune effector cells. In specific embodiments, the nucleic acid encoding the CARs is introduced into the cell using a recombinant vector such as a viral vector including at least a lentivirus, a retrovirus, gamma-retroviruses, an adeno-associated virus (AAV), a herpesvirus, or adenovirus, for example.

CARs can be achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs described herein can also be used for nucleic acid immunization and gene therapy, using standard gene delivery protocols. Methods for gene delivery are known in the art (see, e.g., U.S. Pat. Nos. 5,399,346, 5,580,859, and 5,589,466). In certain embodiments gene therapy vectors are provided.

The nucleic acid encoding the CAR can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

In certain embodiments the expression vector may be provided to a cell in the form of a viral vector. Viral vector technology is well known in the art and is described, for example, in Sambrook et al. (2001) Molecular Cloning: A Laboratory Manual, Cold Spring Harbor Laboratory, New York), and in other virology and molecular biology manuals.

Viruses that are useful as vectors include, but are not limited to, retroviruses, adenoviruses, adeno-associated viruses, herpes viruses, and lentiviruses (including self-inactivating lentivirus vectors). In general, a suitable vector contains an origin of replication functional in at least one organism, a promoter sequence, convenient restriction endonuclease sites, and one or more selectable markers (see, e.g. WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g. enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1alpha (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Moreover, the constructs are not be limited to the use of constitutive promoters and inducible and/or tissue-specific promoters are also contemplated. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Methods of introducing and expressing genes into a cell are known in the art. In the context of an expression vector, the vector can be readily introduced into a host cell, e.g. mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g. Sambrook et al. (2001) Molecular Cloning: A Laboratory Manual, Cold Spring Harbor Laboratory, New York). One illustrative, but non-limiting method for the introduction of a polynucleotide into a host cell is calcium phosphate transfection.

Biological methods for introducing a polynucleotide of interest into a host cell can include the use of DNA and RNA vectors. Viral vectors, and especially retroviral vectors, have become the most widely used method for inserting genes into mammalian, e.g. human cells. Other viral vectors can be derived from lentivirus, poxviruses, herpes simplex virus I, adenoviruses and adeno-associated viruses, and the like (see, e.g. U.S. Pat. Nos. 5,350,674 and 5,585,362, and the like).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An illustrative colloidal system for use as a delivery vehicle in vitro and in vivo is a liposome (e.g., an artificial membrane vesicle).

In the case where a non-viral delivery system is utilized, one illustrative delivery vehicle is a lipid and/or a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In various embodiments lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform can be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al. (1991) Glycobiology 5: 505-510). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

The methods may further comprise culturing cells in media prior to introducing one or more nucleic acids into the cells. The culturing may comprise incubating the selected cells with medium comprising one or more growth factors, in some cases, and the one or more growth factors may comprise c-kit ligand, flt-3 ligand, and/or human thrombopoietin (TPO), for example. The growth factors may or may not be at a certain concentration, such as between about 5 ng/ml to about 500 ng/ml.

In manufacturing the engineered immune effector cells of the present disclosure, the cells may be present in a particular serum-free medium. In specific aspects, the serum-free medium further comprises externally added FLT3 ligand (FLT3L), interleukin 7 (IL-7), stem cell factor (SCF), thrombopoietin (TPO), stem cell factor (SCF), thrombopoietin (TPO), IL-2, IL-4, IL-6, IL-15, IL-21, TNF-alpha, TGF-beta, interferon-gamma, interferon-lambda, TSLP, thymopentin, pleotrophin, midkine, or combinations thereof. The serum-free medium may further comprise vitamins, including biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or combinations thereof or salts thereof. The serum-free medium may further comprise one or more externally added (or not) proteins, such as albumin or bovine serum albumin, a fraction of BSA, catalase, insulin, transferrin, superoxide dismutase, or combinations thereof. The serum-free medium may further comprise corticosterone, D-Galactose, ethanolamine, glutathione, L-carnitine, linoleic acid, linolenic acid, progesterone, putrescine, sodium selenite, or triodo-I-thyronine, or combinations thereof. The serum-free medium may comprise a B-27® supplement, xeno-free B-27® supplement, GS21™ supplement, or combinations thereof. Amino acids (including arginine, cysteine, isoleucine, leucine, lysine, methionine, glutamine, phenylalanine, threonine, tryptophan, histidine, tyrosine, or valine, or combinations thereof), monosaccharides, and/or inorganic ions (including sodium, potassium, calcium, magnesium, nitrogen, or phosphorus, or combinations or salts thereof, for example) may be present in the serum-free medium. The serum-free medium may further comprise molybdenum, vanadium, iron, zinc, selenium, copper, or manganese, or combinations thereof.

Cell culture conditions may be provided for the culture of immune effector cells described herein and for the production of immune effector cells, for example, NK cells, expressing one or more CARs disclosed herein and/or positive/negative selection thereof. In certain aspects, starting cells of a selected population may comprise at least or about 10⁴, 10⁵, 10⁶, 10⁷, 10⁸, 10⁹, 10¹⁰, 10¹¹, 10¹², 10¹³ cells or any range derivable therein. The starting cell population may have a seeding density of at least or about 10, 10¹, 10², 10³, 10⁴, 10⁵, 10⁶, 10⁷, 10⁸ cells/ml, or any range derivable therein.

A culture vessel used for culturing the 3D cell aggregates or progeny cells thereof can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing the stem cells therein. The stem cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, laminin, and fibronectin and mixtures thereof for example Matrigel™, and lysed cell membrane preparations.

Various defined matrix components may be used in the culturing methods or compositions. For example, recombinant collagen IV, fibronectin, laminin, and vitronectin in combination may be used to coat a culturing surface as a means of providing a solid support for pluripotent cell growth, as described in Ludwig et al. (2006a; 2006b), which are incorporated by reference in its entirety.

A matrix composition may be immobilized on a surface to provide support for cells. The matrix composition may include one or more extracellular matrix (ECM) proteins and an aqueous solvent. The term “extracellular matrix” is recognized in the art. Its components include one or more of the following proteins: fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. Other extracellular matrix proteins are described in Kleinman et al., (1993), herein incorporated by reference. It is intended that the term “extracellular matrix” encompass a presently unknown extracellular matrix that may be discovered in the future, since its characterization as an extracellular matrix will be readily determinable by persons skilled in the art.

In some aspects, the total protein concentration in the matrix composition may be about 1 ng/ml to about 1 mg/mL. In some embodiments, the total protein concentration in the matrix composition is about 1 μg/mL to about 300 μg/mL. In more preferred embodiments, the total protein concentration in the matrix composition is about 5 μg/mL to about 200 μg/mL.

The extracellular matrix (ECM) proteins may be of natural origin and purified from human or animal tissues. Alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. In some embodiments, the matrix composition includes synthetically generated peptide fragments of fibronectin or recombinant fibronectin.

In still further embodiments, the matrix composition includes a mixture of at least fibronectin and vitronectin. In some other embodiments, the matrix composition preferably includes laminin.

The matrix composition preferably includes a single type of extracellular matrix protein. In some embodiments, the matrix composition includes fibronectin, particularly for use with culturing progenitor cells. For example, a suitable matrix composition may be prepared by diluting human fibronectin, such as human fibronectin sold by Becton, Dickinson & Co. of Franklin Lakes, N.J. (BD) (Cat #354008), in Dulbecco's phosphate buffered saline (DPBS) to a protein concentration of 5 μg/mL to about 200 μg/mL. In a particular example, the matrix composition includes a fibronectin fragment, such as RetroNectin®. RetroNectin® is a ˜63 kDa protein of (574 amino acids) that contains a central cell-binding domain (type III repeat, 8,9,10), a high affinity heparin-binding domain II (type III repeat, 12,13,14), and CS1 site within the alternatively spliced IIICS region of human fibronectin.

In some other embodiments, the matrix composition may include laminin. For example, a suitable matrix composition may be prepared by diluting laminin (Sigma-Aldrich (St. Louis, Mo.); Cat #L6274 and L2020) in Dulbecco's phosphate buffered saline (DPBS) to a protein concentration of 5 μg/ml to about 200 μg/ml.

In some embodiments, the matrix composition is xeno-free, in that the matrix is or its component proteins are only of human origin. This may be desired for certain research applications. For example in the xeno-free matrix to culture human cells, matrix components of human origin may be used, wherein any non-human animal components may be excluded. In certain aspects, Matrigel™ may be excluded as a substrate from the culturing composition. Matrigel™ is a gelatinous protein mixture secreted by mouse tumor cells and is commercially available from BD Biosciences (New Jersey, USA). This mixture resembles the complex extracellular environment found in many tissues and is used frequently by cell biologists as a substrate for cell culture, but it may introduce undesired xeno antigens or contaminants.

Isolation of immune effector cells include any selection methods, including cell sorters, magnetic separation using antibody-coated magnetic beads, packed columns; affinity chromatography; cytotoxic agents joined to a monoclonal antibody or used in conjunction with a monoclonal antibody, including but not limited to, complement and cytotoxins; and “panning” with antibody attached to a solid matrix, e.g., plate, or any other convenient technique.

The use of separation or isolation techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye Hoechst 33342). Techniques providing accurate separation include but are not limited to, FACS (Fluorescence-activated cell sorting) or MACS (Magnetic-activated cell sorting), which can have varying degrees of sophistication, e.g., a plurality of color channels, low angle and obtuse light scattering detecting channels, impedance channels, etc.

The antibodies utilized in the preceding techniques or techniques used to assess cell type purity (such as flow cytometry) can be conjugated to identifiable agents including, but not limited to, enzymes, magnetic beads, colloidal magnetic beads, haptens, fluorochromes, metal compounds, radioactive compounds, drugs or haptens. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies, see Haugland, Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxygenin, oxazalone, and nitrophenol. The radioactive compounds that can be conjugated or incorporated into the antibodies are known to the art, and include but are not limited to technetium 99m (99TC), 1251 and amino acids comprising any radionuclides, including, but not limited to, 14C, 3H and 35S.

Other techniques for positive selection may be employed, which permit accurate separation, such as affinity columns, and the like. The method should permit the removal to a residual amount of less than about 20%, preferably less than about 5%, of the non-target cell populations.

Cells may be selected based on light-scatter properties as well as their expression of various cell surface antigens. The purified stem cells have low side scatter and low to medium forward scatter profiles by FACS analysis. Cytospin preparations show the enriched stem cells to have a size between mature lymphoid cells and mature granulocytes.

Various techniques may be employed to separate the cells by initially removing cells of dedicated lineage. Monoclonal antibodies are particularly useful for identifying markers associated with particular cell lineages and/or stages of differentiation. The antibodies may be attached to a solid support to allow for crude separation. The separation techniques employed should maximize the retention of viability of the fraction to be collected. Various techniques of different efficacy may be employed to obtain “relatively crude” separations. Such separations are where up to 10%, usually not more than about 5%, preferably not more than about 1%, of the total cells present are undesired cells that remain with the cell population to be retained. The particular technique employed will depend upon efficiency of separation, associated cytotoxicity, case and speed of performance, and necessity for sophisticated equipment and/or technical skill.

Selection of the progenitor cells need not be achieved solely with a marker specific for the cells. By using a combination of negative selection and positive selection, enriched cell populations can be obtained.

In certain embodiments, cells containing an exogenous nucleic acid may be identified in vitro or in vivo by including a marker in the expression vector or the exogenous nucleic acid. Such markers would confer an identifiable change to the cell permitting easy identification of cells containing the expression vector. Generally, a selection marker may be one that confers a property that allows for selection. A positive selection marker may be one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abier gene or Neo gene from Tn5, which confers antibiotic resistance to geneticin.

A screenable marker may comprise a reporter gene, which allows the researcher to distinguish between wanted and unwanted cells. Certain embodiments of the present disclosure utilize reporter genes to indicate specific cell lineages. For example, the reporter gene can be located within expression elements and under the control of the ventricular- or atrial-selective regulatory elements normally associated with the coding region of a ventricular- or atrial-selective gene for simultaneous expression. A reporter allows the cells of a specific lineage to be isolated without placing them under drug or other selective pressures or otherwise risking cell viability.

Examples of such reporters include genes encoding cell surface proteins (e.g., CD4, HA epitope), fluorescent proteins, antigenic determinants and enzymes (e.g., ß-galactosidase). The vector containing cells may be isolated, e.g., by FACS using fluorescently-tagged antibodies to the cell surface protein or substrates that can be converted to fluorescent products by a vector encoded enzyme.

In specific embodiments, the reporter gene is a fluorescent protein. A broad range of fluorescent protein genetic variants have been developed that feature fluorescence emission spectral profiles spanning almost the entire visible light spectrum. Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission. Developmental research efforts are ongoing to improve the brightness and stability of fluorescent proteins, thus improving their overall usefulness.

The cells in certain embodiments can be made to contain one or more genetic alterations by genetic engineering of the cells either before or after differentiation (US 2002/0168766). A cell is said to be “genetically altered”, “genetically modified” or “transgenic” when an exogenous nucleic acid or polynucleotide has been transferred into the cell by any suitable means of artificial manipulation, or where the cell is a progeny of the originally altered cell that has inherited the polynucleotide. For example, the cells can be processed to increase their replication potential by genetically altering the cells to express telomerase reverse transcriptase, either before or after they progress to restricted developmental lineage cells or terminally differentiated cells (U.S. Patent Application Publication 2003/0022367).

In certain embodiments, cells containing an exogenous nucleic acid construct may be identified in vitro or in vivo by including a marker in the expression vector, such as a selectable or screenable marker. Such markers would confer an identifiable change to the cell permitting easy identification of cells containing the expression vector, or help enrich or identify differentiated cardiac cells by using a tissue-specific promoter. For example, in the aspects of cardiomyocyte differentiation, cardiac-specific promoters may be used, such as promoters of cardiac troponin I (cTnI), cardiac troponin T (cTnT), sarcomeric myosin heavy chain (MHC), GATA-4, Nkx2.5, N-cadherin, B1-adrenoceptor, ANF, the MEF-2 family of transcription factors, creatine kinase MB (CK-MB), myoglobin, or atrial natriuretic factor (ANF). In aspects of neuron differentiation, neuron-specific promoters may be used, including but not limited to, TuJ-1, Map-2, Dcx or Synapsin. In aspects of hepatocyte differentiation, definitive endoderm- and/or hepatocyte-specific promoters may be used, including but not limited to, ATT, Cyp3a4, ASGPR, FoxA2, HNF4a or AFP.

Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to blasticidin, neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

In embodiments wherein cells are genetically modified, such as to add or reduce one or more features, the genetic modification may occur by any suitable method. For example, any genetic modification compositions or methods may be used to introduce exogenous nucleic acids into cells or to edit the genomic DNA, such as gene editing, homologous recombination or non-homologous recombination, RNA-mediated genetic delivery or any conventional nucleic acid delivery methods. Non-limiting examples of the genetic modification methods may include gene editing methods such as by CRISPR/CAS9, zinc finger nuclease, or TALEN technology.

Genetic modification may also include the introduction of a selectable or screenable marker that aid selection or screen or imaging in vitro or in vivo. Particularly, in vivo imaging agents or suicide genes may be expressed exogenously or added to starting cells or progeny cells. In further aspects, the methods may involve image-guided adoptive cell therapy.

### VII. Methods of Using the Cells

The immune effector cells of the disclosure may or may not be utilized directly after production. In some cases they are stored for later purpose. In any event, they may be utilized in therapeutic or preventative applications for a mammalian subject (human, dog, cat, horse, etc.) such as a patient. The individual may be in need of cell therapy for a medical condition of any kind, including allogeneic cell therapy.

Methods of treating an individual with a therapeutically effective amount of immune effector cells of the disclosure comprise administering the cells or clonal populations thereof to the patient. The cells or cell populations may be allogeneic with respect to the patient. The individual does not exhibit signs of depletion of the cells or cell population, in particular embodiments. The individual may or may not have cancer and/or a disease or condition involving inflammation. In specific embodiments wherein the individual has cancer, tumor cells of the cancer patient are killed after administering the cells or cell population to the individual. In specific cases wherein the patient has inflammation, the inflammation is reduced following administering the cells or cell population to the patient.

For individuals with cancer, once infused into the individuals it is expected that this cell product can employ multiple mechanisms to target and eradicate tumor cells. The infused cells can directly recognize and kill tumor antigen-expressing cells through granzyme- and FasL-mediated cytotoxicity. In some embodiments, the tumor antigen expressed by cancer and/or tumors is MUC18⁺. The infused cells can also directly recognize and kill stress ligand-expressing cells through granzyme- and FasL-mediated cytotoxicity. In some embodiments, the stress ligand expressed by immunosuppressive cells in the TME is an NKG2D ligand.

In some embodiments, disclosed are methods for stimulating an immune cell-mediated immune response to a target cell population and/or tissue in a mammal, wherein the target cell population and/or tissue express MUC18 and/or one or more NKG2D ligands, the method comprising administering to a mammal in need thereof a therapeutically effective amount of a composition comprising an immune effector cell expressing two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof.

In some embodiments, disclosed are methods of generating a persisting population of genetically engineered immune cells in a mammal, said method comprising administering to the mammal a composition comprising immune effector cells genetically modified to express two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof, wherein the persisting population of genetically engineered immune cells persists in the mammal for at least about one month up to at least about one year after administration. In some embodiments, the immune cell population may persist for about one month to about 11 months, about one month to about 10 months, about 1 month to about nine months, about one month to about eight months, about one month to about seven months, about one month to about six months, about one month to about five months, about one month to about four months, about one month to about three months, or about one month to about two months, for example.

In some embodiments, disclosed are methods of expanding a population of genetically engineered immune cells in a mammal, the method comprising administering to the mammal a composition comprising immune effector cells genetically modified to express two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof, wherein the administered genetically engineered immune cell produces a population of progeny immune cells in the mammal.

In some embodiments, disclosed are methods of treating a mammal with a cancer comprising cells that express MUC18 and/or one or more NKG2D ligands, the method comprising administering to a mammal in need thereof a therapeutically effective amount of a composition comprising immune effector cells genetically modified to express two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof.

In some embodiments, disclosed are methods of treating cancer comprising the steps of contacting an immune effector cell with a cancer cell or MDSC, M2-TAM, or Treg within the cancer microenvironment of a mammal and inducing apoptosis of the cancer cell, wherein the immune effector cell is genetically modified to express two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof.

In certain embodiments the CAR-modified cells described herein can also serve as a type of vaccine for ex vivo immunization and/or in vivo therapy in a mammal. In certain embodiments the mammal is a non-human mammal and in other embodiments the mammal is a human.

With respect to ex vivo immunization, at least one of the following can occur in vitro prior to administering the cell into a mammal: i) expansion of the cells, ii) introducing a nucleic acid encoding a CAR to the cells, and/or iii) cryopreservation of the cells.

Ex vivo procedures are well known in the art and are discussed more fully below. Briefly, cells are isolated from a mammal (preferably a human) and genetically modified (i.e., transduced or transfected in vitro) with a vector expressing a CAR disclosed herein. The CAR-modified cell can be administered to a mammalian recipient to provide a therapeutic benefit. In certain embodiments the CAR-modified cell can be autologous with respect to the recipient. Alternatively, the cells can be allogeneic, syngeneic or xenogeneic with respect to the recipient.

In specific cases, the present therapy is useful for individuals with cancers that have been clinically indicated to be subject to immune effector cell regulation, including multiple types of solid tumors (melanoma, colon, lung, breast, and head and neck cancers), for example.

Individuals treated with the present cell therapy may or may not have been treated for the particular medical condition prior to receiving the immune effector cell therapy. In cases wherein the individual has cancer, the cancer may be primary, metastatic, resistant to therapy, and so forth.

In particular embodiments, the cells are provided to the patient at 10⁷-10⁹ cells per dose. In specific embodiments, the dosing regimen is a single-dose of immune effector cell. The immune effector cells may or may not be allogenic to the individual. The cells may be administered intravenously or by any other suitable route known in the art. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

Methods may be employed with respect to individuals who have tested positive for a medical condition, who have one or more symptoms of a medical condition, or who are deemed to be at risk for developing such a condition. In some embodiments, the compositions and methods described herein are used to treat an inflammatory or autoimmune component of a disorder listed herein and/or known in the art.

In some embodiments, the methods relate to administration of the cells or compositions described herein for the treatment of a cancer or administration to a person with a cancer. In some embodiments, the method is for a patient with a solid tumor. In some embodiments, the solid tumor is sarcoma and/or carcinoma. In some embodiments, the sarcoma is rhabdomyosarcoma, Ewing sarcoma, clear cell sarcoma, or leiomyosarcoma. In some embodiments, the carcinoma is melanoma or prostatic adenocarcinoma.

In some embodiments, the patient has received at least 1, 2, 3, 4, 5, 6, 7, 8, or more prior treatments for a solid tumor. The prior treatments may include a treatment or therapy described herein. In some embodiments, the prior treatments comprise conventional chemotherapy, conventional radiotherapy, anti-PDL1, or anti-CTLA4 therapy. In some embodiments, the patient had received the prior therapy within 10, 20, 30, 40, 50, 60, 70, 80, or 90 days or hours of administration of the current compositions and cells of the disclosure. In some embodiments, the patient is one that has undergone prior therapy and has failed the prior treatment either because the prior treatment was not effective or because the prior treatment was deemed too toxic.

In some embodiments, the individual is one in which at least 5, 6, 7, 8, 9, 10, 11, 12, 13, 14, 15, 16, 17, 18, 19, 20, 21, 22, 23, 24, 25, 26, 27, 28, 39, or 30% of the malignant cells or malignant plasma cells express MUC18. In some embodiments, the patient is one that has been determined to have MUC18+ malignant cells.

In some embodiments, the methods and compositions may be for vaccinating an individual to prevent a medical condition, such as cancer.

### VIII. Cancer Therapy

The cancer of the disclosed methods can be any cell in a subject undergoing unregulated growth, invasion, or metastasis. Cancers include solid tumor cancers, for example, sarcomas and carcinomas. Sarcomas can include rhabdomyosarcoma, Ewing sarcoma, clear cell sarcoma, or leiomyosarcoma. Carcinomas can include melanoma or prostatic adenocarcinoma. Cancers also include prostate cancer, ovarian cancer, adenocarcinoma of the lung, breast cancer, endometrial cancer, gastric cancer, colon cancer, and pancreatic cancer.

In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed immune effector cells expressing the first and second CARs can be used in combination with other cancer therapies and/or any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy. The disclosed CARs can be used in combination with a checkpoint inhibitor.

The disclosed immune effector cells expressing the first and second CARs can be used in combination with other cancer therapies. In some embodiments, the method further comprises administering a cancer therapy to the patient. The cancer therapy may be chosen based on the expression level measurements, alone or in combination with the clinical risk score calculated for the patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. Any of these cancer therapies may also be excluded. Combinations of these therapies may also be administered. In some embodiments, the gene or miRNA expression measurement and analysis may indicate that one or more cancer therapies would be likely to be effective or ineffective. A particular advantage of methods disclosed herein is that they allow doctors for the first time to make a treatment decision based on the molecular subtype of a metastasis.

In some embodiments, additional therapies include immunotherapy; oncolytic virus; polysaccharides; neoantigens; chemotherapy; radiotherapy; surgery; and other agents.

### A. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

### 1. Checkpoint Inhibitors and Combination Treatment

Embodiments of the disclosure may include administration of immune checkpoint inhibitors (also referred to as checkpoint inhibitor therapy), which are further described below. The checkpoint inhibitor therapy may be a monotherapy, targeting only one cellular checkpoint proteins or may be combination therapy that targets at least two cellular checkpoint proteins. For example, the checkpoint inhibitor monotherapy may comprise one of: a PD-1, PD-L1, or PD-L2 inhibitor or may comprise one of a CTLA-4, B7-1, or B7-2 inhibitor. The checkpoint inhibitor combination therapy may comprise one of: a PD-1, PD-L1, or PD-L2 inhibitor and, in combination, may further comprise one of a CTLA-4, B7-1, or B7-2 inhibitor. The combination of inhibitors in combination therapy need not be in the same composition, but can be administered either at the same time, at substantially the same time, or in a dosing regimen that includes periodic administration of both of the inhibitors, wherein the period may be a time period described herein.

a. PD-1, PD-L1, and PD-L2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect, the PD-1 ligand binding partners are PD-L1 and/or PD-L2. In another embodiment, a PD-L1 inhibitor is a molecule that inhibits the binding of PD-L1 to its binding partners. In a specific aspect, PD-L1 binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-L1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PD-L1, or PD-L2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2 Inhibitors

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA-4 is a member of the immunoglobulin superfamily that is expressed on the surface of Helper T cells and transmits an inhibitory signal to T cells. CTLA-4 is similar to the T-cell costimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1. B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### 2. Inhibition of Costimulatory Molecules

In some embodiments, the immunotherapy comprises an inhibitor of a costimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment, they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNΔ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### B. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

### C. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### D. Neoantigens

In some embodiments, the additional therapy comprises neoantigen administration. Many tumors express mutations. These mutations potentially create new targetable antigens (neoantigens) for use in T cell immunotherapy. The presence of CD8⁺ T cells in cancer lesions, as identified using RNA sequencing data, is higher in tumors with a high mutational burden. The level of transcripts associated with cytolytic activity of natural killer cells and T cells positively correlates with mutational load in many human tumors.

### E. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN₂), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### F. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IX. Kits

Certain aspects of the present disclosure also concern kits containing compositions of the disclosure or compositions to implement methods of the disclosure. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for methylation of one or more biomarkers. In some embodiments, a control includes a nucleic acid that contains at least one CpG or is capable of identifying a CpG methylation site.

It is contemplated that any method or composition described herein can be implemented with respect to any other method or composition described herein and that different embodiments may be combined. The claims originally filed are contemplated to cover claims that are multiply dependent on any filed claim or combination of filed claims.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: NK Cells Expressing a Chimeric Activating Receptor Eliminate MDSCS and Rescue Impaired Car-T Cell Activity Against Solid Tumors

To overcome the repressive effect of the solid TME on NKG2D function, the inventors used a retroviral vector to modify NK cells with a chimeric NKG2D receptor (NKG2D.ζ) comprising the extracellular domain of the native NKG2D molecule fused to the intracellular cytotoxic-chain of the T-cell receptor (16). The inventors hypothesized that primary human NK cells expressing NKG2D.ζ (NKG2D.ζ-NK cells) would maintain NKG2D.ζ expression within the suppressive TME, kill NKG2D ligand-expressing MDSCs, secrete proinflammatory cytokines and chemokines, and recruit and activate effector cells, including CAR-T cells, derived from the adaptive immune system. These benefits are not attainable from NK cells expressing the native NKG2D receptor as its functions are downmodulated in the TME. The inventors show that when NK cells express NKG2D.ζ, immune suppression is sufficiently countered to enable tumor-specific CAR-T cells to persist within the TME and eradicate otherwise resistant tumors.

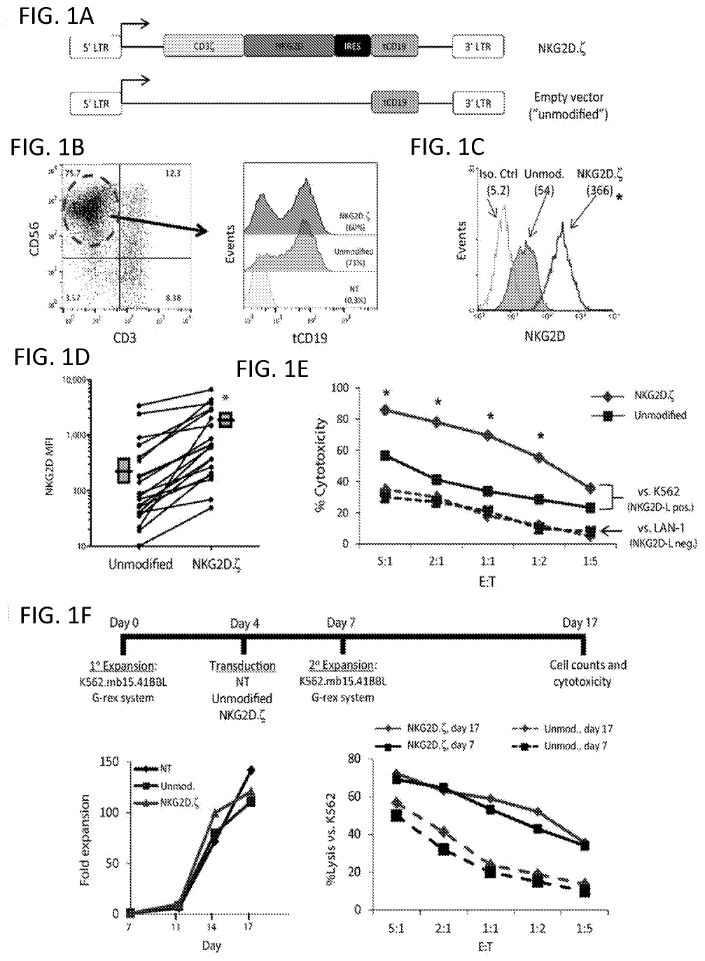

NKG2D.ζ NK cells expand and have cytotoxicity against target cells. To increase killing of NKG2D ligand-expressing MDSCs, the inventors generated primary human NK cells stably expressing NKG2D.ζ and a truncated CD19 (tCD19) marker from a retroviral vector (FIG. 1A). NK cells were expanded from PBMCs obtained from normal donors, transduced with retroviral construct expressing chimeric NKG2D, then cultured for 3 additional days. Transduction efficiency, as measured by the expression of tCD19 on CD56⁺CD3⁻ NK cells after the additional 3 days, was 71.3%±16% (n=25 normal donors) and produced a 5.4±1.1-fold increase in NKG2D expression on the NK cell surface (FIGS. 1B-1D). NKG2D.ζ-NK cells showed greater cytotoxicity (79.2%±5.6%, n=10 normal donors) against wild-type K562, a highly NK cell-sensitive tumor cell line that naturally expresses NKG2D ligands, than mock vector-transduced (hereafter referred to as unmodified) NK cells (40.5%±2.1%) at 2:1 E:T ratio in a 4-hour cytotoxicity assay (FIG. 1E). In contrast, transgenic NKG2D.ζ expression did not increase NK cell killing of LAN-1 neuroblastoma cells that are marginally NK sensitive, but lack NKG2D ligands. To determine if in vitro expansion affected the cytotoxic function of NKG2D.ζ-NK cells, the inventors secondarily expanded NKG2D.ζ-NK cells for an additional 10 days (FIG. 1F, schema). As seen in FIG. 1F. NKG2D.ζ-NK cells expanded (120±7.3-fold by day 17 of culture; n=10 donors) similarly to unmodified and nontransduced NK cells and maintained stable cytotoxic function between days 7 and 17 of expansion. Thus, the inventors generated and expanded high numbers of primary human NKG2D.ζ-expressing NK cells capable of cytotoxicity against ligand-expressing targets, even after prolonged culture.

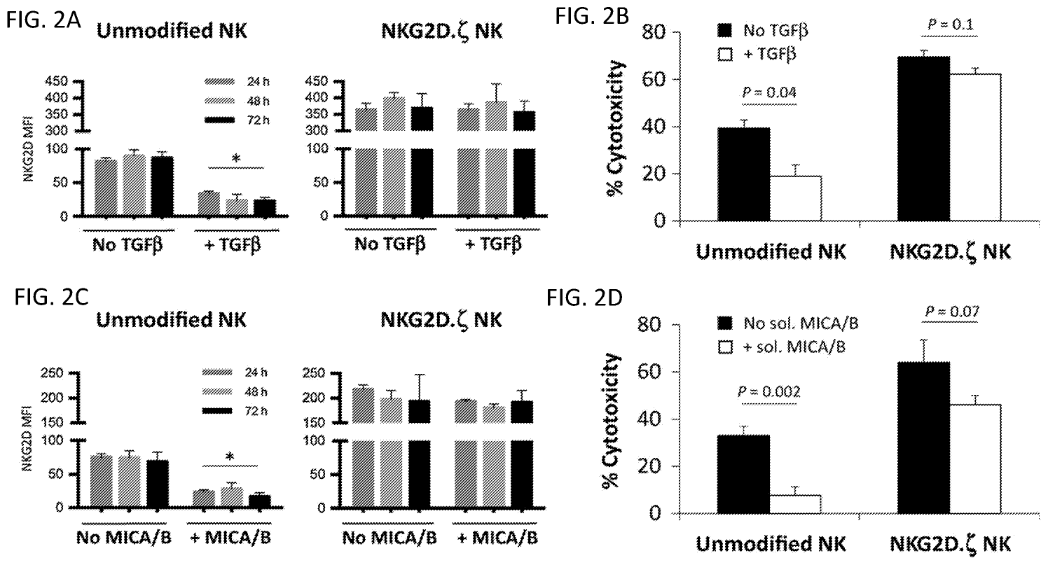

Transgenic NKG2D.ζ is unaffected by TGFβ or soluble NKG2D ligands. Expression of the native NKG2D receptor on NK cells is downmodulated by tumor-derived TGFβ and soluble NKG2D ligands, both of which are abundant in the TME (15, 29) and likely impair NK cell function in solid tumors. To determine the effect of TGFβ and soluble NKG2D ligands on NKG2D.ζ receptor expression and function, the inventors cultured NKG2D.ζ-NK cells in the presence of TGFβ or the soluble NKG2D ligands, MICA and MICB, and examined NKG2D expression and NK cytotoxicity after 24, 48, and 72 hours. After exposure to TGFβ or soluble MICA/B, unmodified NK cells significantly downregulated NKG2D (MFI of 25 vs. 95 in nonexposed NK cells at 48 hours) and were less cytotoxic (20%±5.1% killing vs. 40%±3.7% killing by nonexposed NK cells at 48 hours) to NKG2D ligand-expressing K562 targets (FIGS. 2A and 2B). In contrast. NKG2D.ζ-NK cells maintained NKG2D expression and cytotoxicity after exposure to the same concentrations of TGFβ and soluble MICA/B (FIGS. 2C and 2D). This lack of sensitivity to downregulation by these tumor-associated components should benefit the function of NKG2D.ζ-NK cells within the TME.

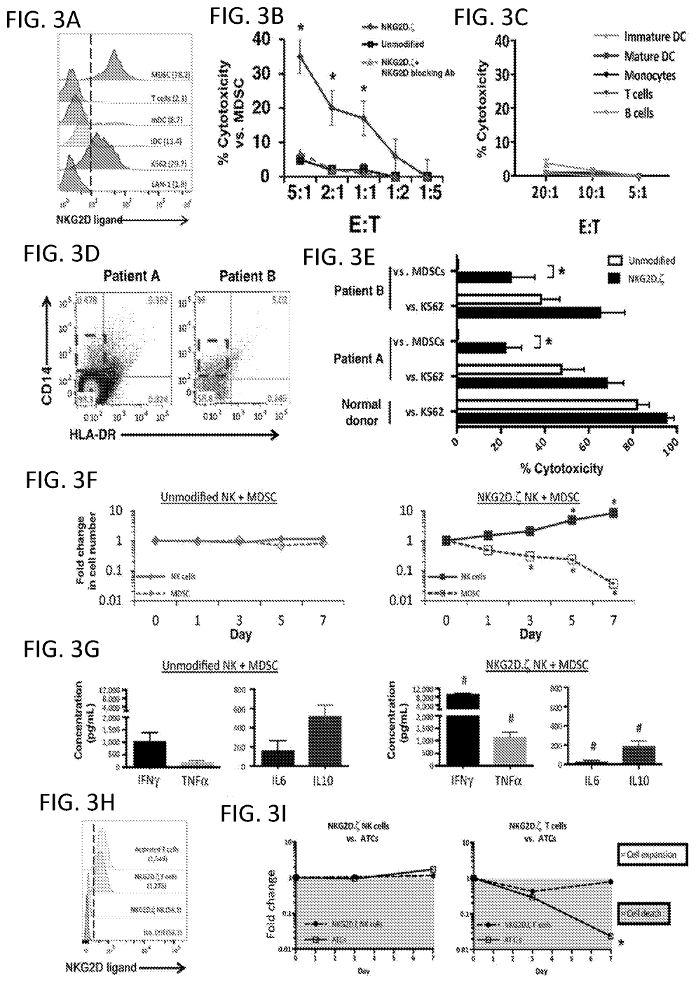

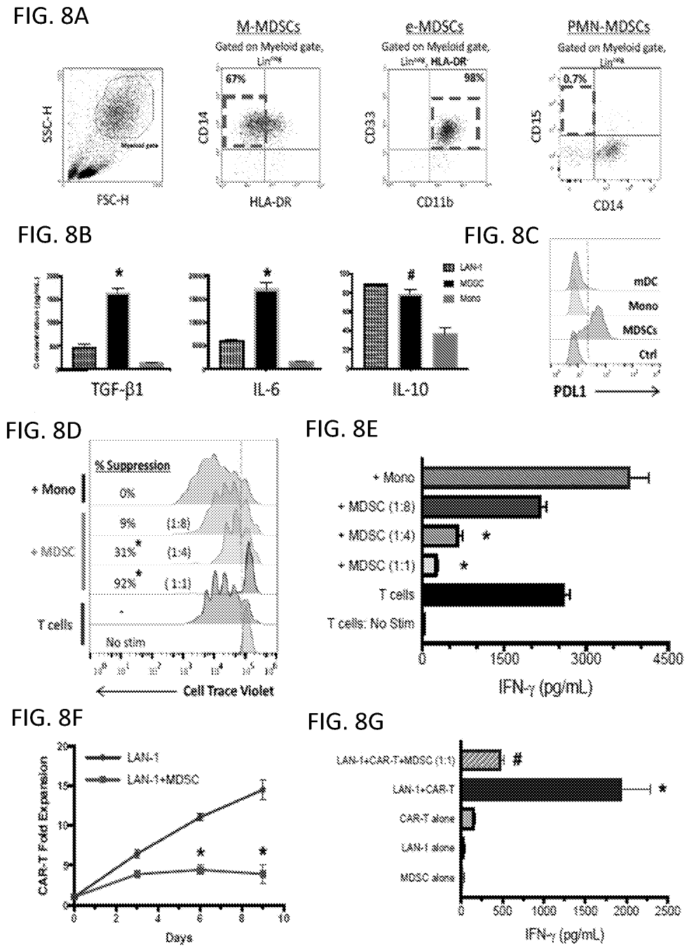

Human MDSCs express NKG2D ligands and are killed by NKG2D.ζ-NK cells. To study the effects of human NK cells on autologous MDSCs, the inventors generated human MDSCs by culture of CD3⁻/CD25^{lo} PBMC with IL6 plus GM-CSF for 7 days, followed by CD33⁺ selection, as described in Materials and Methods. The phenotypic characterization of these MDSCs and confirmation of their suppressive capacity are shown in FIG. 8. Routinely, the inventors' ex vivo-generated MDSCs contained monocytic (M)-MDSC and early (c)-MDSC subsets, with few (average <1%) polymorphonuclear (PMN)-MDSCs (FIG. 8A), roughly reflecting the subset composition reported in patients with solid tumors (9, 30). The MDSCs expressed the suppressive factors TGFβ. IL6. IL10, and PDL-1 in amounts often greater than tumor cells (FIGS. 8B and 8C) and suppressed proliferation and cytokine secretion by autologous T cells stimulated with plate-bound CD3/CD28 antibodies (FIGS. 8D and 8E) and by second-generation GD2.CAR-T cells encoding 4-1BB and CD3-endodomains stimulated with the GD2+ tumor line LAN-1 (FIGS. 8F and 8G). As seen in FIG. 3A. MDSCs expressed as much or more NKG2D ligand than the positive control tumor line K562 (ligand MFI of 78.2 vs. 29.7, respectively). Freshly isolated peripheral blood T cells did not express NKG2D ligands, whereas immature and mature dendritic cells expressed little, consistent with previous data (13). The neuroblastoma cell line LAN-1, subsequently used in the inventors' in vivo TME model, did not express NKG2D ligands.

To evaluate MDSC susceptibility to killing by NKG2D.ζ-NK cells, the inventors performed both short- and long-term killing assays. FIG. 3B shows enhanced killing of MDSCs by autologous NKG2D.ζ-NK cells compared with unmodified NK cells (35%±5.5% vs. 8%±2.4% cytotoxicity, respectively, at an E:T ratio of 5:1) in a 4-hour chromium-release assay. MDSC killing was dependent on NKG2D, as preincubation with an NKG2D-blocking Ab reduced the cytotoxicity to levels achieved by unmodified NK cells. NKG2D.ζ-NK cells mediated no cytotoxicity against other autologous immune cells such as freshly isolated monocytes, monocyte-derived mature dendritic cells, T cells, or B cells (FIG. 3C). Only immature dendritic cells, which expressed little NKG2D ligand (approximately 7% of cells; MFI 11.4), were mildly susceptible to lysis by NKG2D.ζ-NK cells (4.2%±1.7% lysis at an E:T ratio of 20:1). As confirmation of the clinical applicability of the inventors' approach, the inventors assessed whether NKG2D.ζ-NK cells generated from patient PBMCs were able to kill highly suppressive MDSCs isolated from the patient's tumor. Tumor samples obtained from two patients with high-risk neuroblastoma at the time of first biopsy/resection contained M-MDSCs (FIG. 3D). NKG2D.ζ-NK cells generated from patient PBMCs (harvested and frozen at time of tumor sampling) mediated significant cytotoxicity in vitro against M-MDSCs purified from patient tumors, whereas unmodified patient NK cells did not (FIG. 3E). These results provide further clinical evidence for the capacity of NKG2D.ζ-NK cells to eliminate MDSCs in patients with suppressive TMEs.

To determine whether NKG2D.ζ-NK cells could control MDSC survival in long-term cultures, the inventors cocultured NKG2D.ζ-NK cells with autologous MDSCs at a 1:1 ratio for 7 days in the presence of low-dose IL2 to maintain NK survival and quantified each cell type by flow cytometry every 2 days. As shown in FIG. 3F. NKG2D.ζ-NK cells expanded in cocultures (mean 9.5±0.7-fold increase) with a concomitant reduction in MDSCs (mean 81.3±9.4-fold decrease), whereas unmodified NK cells failed to expand or eliminate MDSCs. NK cells cultured alone or with autologous monocyte controls did not expand (0.8±0.1-fold change). As seen in FIG. 3G, NK cell expansion and MDSC reduction correlated with a shift in the culture cytokine milieu from one that is immune-suppressive (more IL6 and IL10; less IFNγ and TNFα) in cocultures containing unmodified NK cells, to one that is immune stimulatory and enhances CAR-T antitumor function (less IL6 and IL10; more IFNγ and TNFα) in cocultures containing NKG2D.ζ-NK cells. Hence, NKG2D.ζ-NK cells mediate potent cytotoxicity against suppressive MDSCs via their highly expressed NKG2D ligands. In addition, through selective depletion of MDSCs in combination with immune-stimulatory cytokine secretion. NKG2D.ζ-NK cells skew the cytokine microenvironment to one that can support CAR-T effector functions (31).

Previous studies have reported that expression of chimeric NKG2D constructs in T lymphocytes can direct these cells to target NKG2D ligand expressing tumors (16, 32). However, activated T cells (ATC) themselves upregulate NKG2D ligands (33), with variable ligand expression intensity dependent on the T-cell activation protocol used, leading to fratricide when the chimeric NKG2D is expressed. To determine if this off-tumor side effect occurred when the same NKG2D.ζ was expressed in NK cells, the inventors compared the killing of ATCs by autologous NK cells or by autologous T cells expressing the inventors' NKG2D.ζ transgene. ATCs and NKG2D.ζ-T cells both upregulated NKG2D ligands during ex vivo expansion with CD3/CD28 antibodies plus IL7 and IL15, whereas NKG2D.ζ-transduced NK cells undergoing expansion in the inventors' K562-mb15-41BB-L culture system did not (FIG. 3H). Coculture without additional stimulation of NKG2D.ζ-T cells with autologous ATCs produced fratricide, of both the NKG2D.ζ effector T cells (35±7.2% decrease in cell number) and the nontransduced ATC targets (98%±11.5% decrease in cell number; n=3). By contrast, ATC numbers were unaffected by coculture with autologous NKG2D.ζ-NK cells (FIG. 3I). These results show that NK cells expressing NKG2D, can kill autologous MDSCs while sparing other NKG2D ligand-expressing populations, thus avoiding the fratricide seen with NKG2D.ζ-expressing T cells.

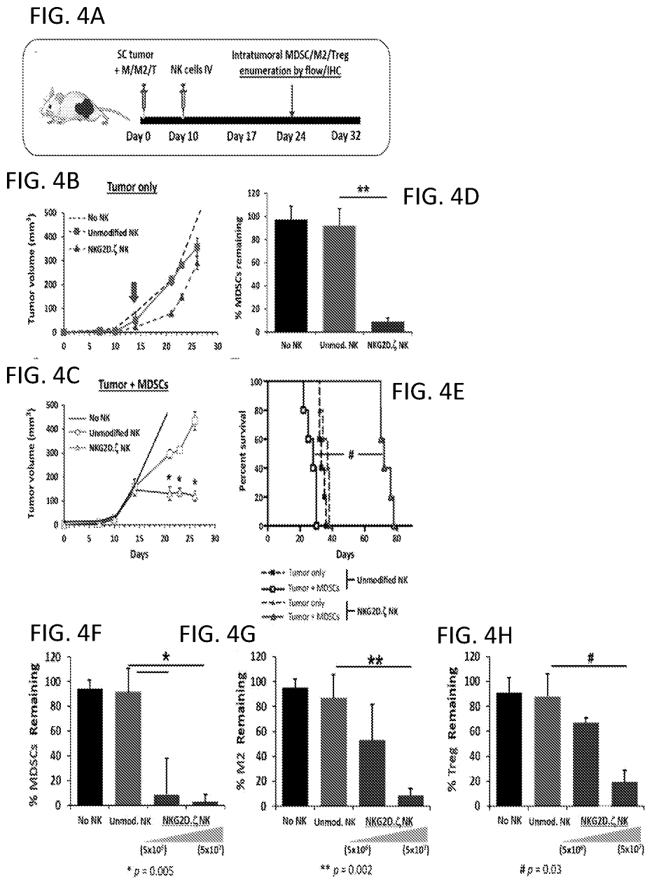

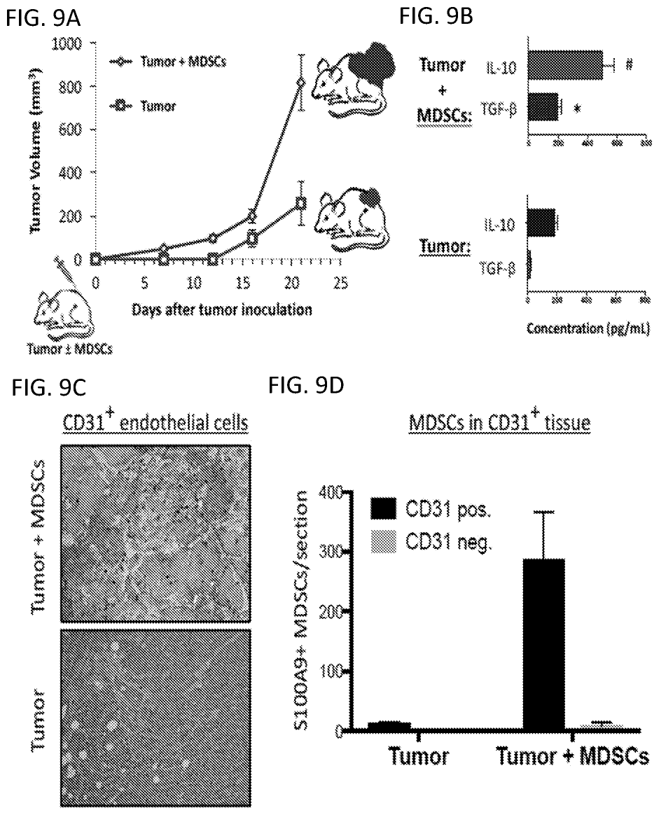

NKG2D.ζ-NK cells eliminate intratumoral MDSCs and reduce tumor burden. To determine if NKG2D.ζ-NK cells could eliminate MDSCs from tumor sites in vivo, the inventors created an MDSC-containing TME in a xenograft model of neuroblastoma. The inventors chose NKG2D ligand-negative LAN-1 tumor for this experiment so that the effects of NKG2D.ζ-NK cells on MDSCs were not confused with their effects on the tumor cells. LAN-1 tumor cells admixed with human MDSCs were inoculated subcutaneously in NSG mice. These animals had increases in the suppressive cytokines IL10 (10-fold vs. tumor alone) and TGFβ (2.6-fold vs. tumor alone) in circulation by day 16 as compared with animals bearing tumors initiated without MDSCs, and the resultant tumors grew more rapidly due to increased neovascularization and tumor-associated stroma (FIGS. 9A-9D), consistent with clinical reports of MDSC-dense tumors (34). FIG. 4A provides an overview of the experimental timeline. As seen in FIG. 4B, in mice bearing NKG2D ligand-negative tumors without MDSCs, a single infusion of 1× 10⁷ NKG2D.ζ-NK cells resulted in a small delay in tumor growth but eventual progression, suggesting that the LAN-1 tumor itself (a marginally NK-sensitive target) can be killed at higher NK cell doses independent of NKG2D ligand expression. FIGS. 4F-4H show that NKG2D.ζ-NK deplete intra-tumoral MDSC. M2, and Treg in vivo. In mice bearing MDSC-containing tumors, 1×10⁷ NKG2D.ζ-NK cells inhibited tumor growth (FIG. 4C), reduced NKG2D ligand-expressing intratumoral MDSCs with only 8.7%±3.5% of the input MDSCs remaining (FIG. 4D), and prolonged mouse survival (median survival of 73 days vs. 29 days after unmodified NK cells; FIG. 4E). Because LAN-1 tumor cells do not express NKG2D ligands and are only marginally sensitive to ligand-independent lysis, tumors subsequently regrew in these mice once the NKG2D.ζ-NK cells had disappeared (>day 40). Thus, NKG2D.ζ-NK cells can traffic to tumor sites and reduce intratumoral MDSCs but cannot themselves eradicate NKG2D ligand-negative malignant cells in the inventors' model.

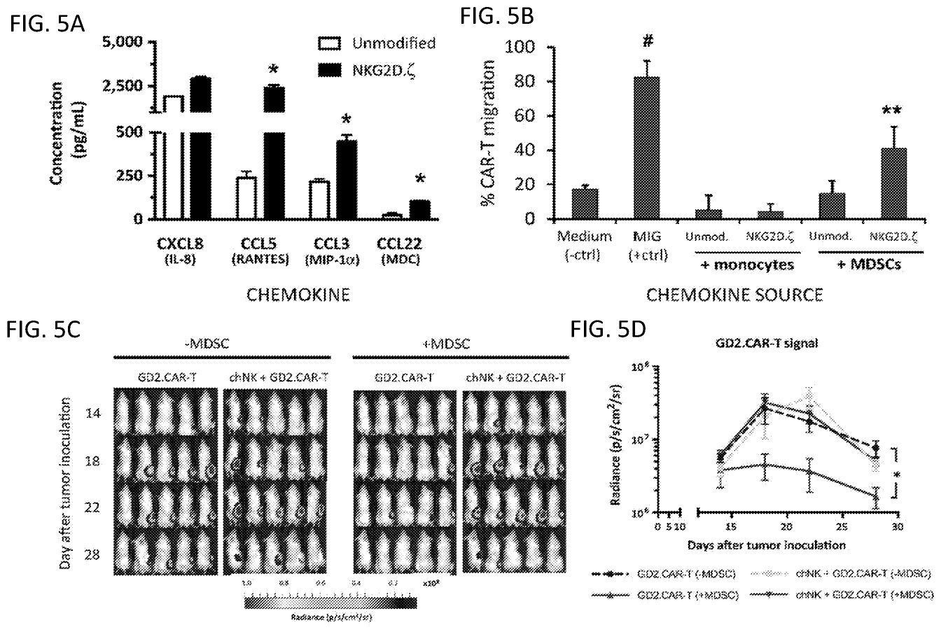

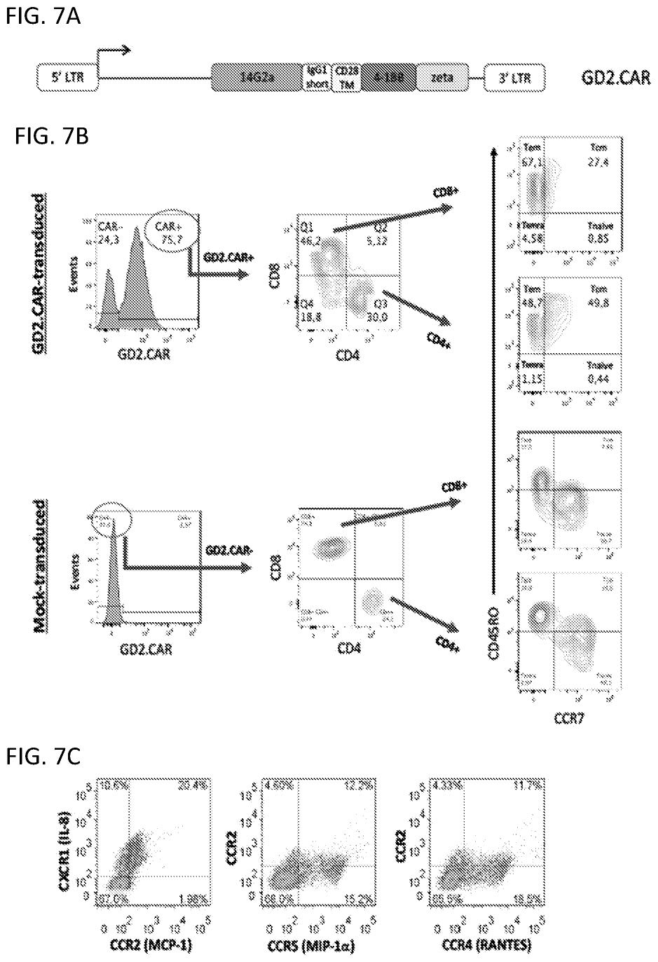

NKG2D.ζ-NK cells secrete chemokines that recruit GD2.CAR-T cells. To determine if NKG2D.ζ-NK cells can recruit T cells modified with a tumor-specific CAR to tumor sites containing MDSCs, the inventors cocultured NKG2D.ζ-NK cells with autologous MDSCs and analyzed culture supernatants for chemokines by multiplex ELISA. Compared with unmodified NK cells, NKG2D.ζ-NK cells produce significantly greater CCL5 (RANTES; 10-fold increase), CCL3 (MIP-1a; 2-fold increase), and CCL22 (MDC; 5-fold increase) in response to autologous MDSCs (FIG. 5A). Large amounts of CXCL8 (IL8) were also produced, but there was no significant difference from the production by unmodified NK cells. Analysis of chemokine receptor expression on second-generation GD2.CAR-T cells revealed CXCR1 (binds CXCL8), CCR2 (binds CCL2), CCR5 (binds CCL3), and CCR4 (binds CCL5; see FIG. 7C). These GD2.CAR-T cells were assayed for chemotaxis to supernatants derived from unmodified or NKG2D.ζ-NK cells cocultured with autologous MDSCs. Supernatants from NKG2D.ζ-NK cell-containing cocultures induced chemotaxis of 41.1%±5.5% of GD2.CAR-T cells (FIG. 5B), whereas supernatants from unmodified NK cells induced chemotaxis no greater than produced by medium (14.9%±6.4% vs. 17.3%±1.9%, respectively). Chemotaxis was not induced by supernatants from unmodified or NKG2D.ζ-NK cells cocultured with monocytes. Thus, following their encounter with MDSCs, NKG2D.ζ-NK cells secrete chemokines that recruit CAR-Ts in vitro.

NKG2D.ζ NK cells improve GD2.CAR-T cell trafficking to tumor sites. To determine the effects of the MDSC-induced, NKG2D.ζ-NK cell chemokines on CAR-T cell recruitment in vivo, the inventors used an MDSC-containing TME xenograft model (see FIG. 4). When tumors reached a volume of ˜100 mm³ (day 10), 5×10⁶ NKG2D.ζ-NK cells were infused, followed 3 days later (day 13) by infusion of 5×10⁶ luciferase gene-transduced GD2.CAR-T cells. Tumor localization and expansion of GD2.CAR-T cells were measured over time via live-animal bioluminescence imaging. As seen in FIG. 5C, GD2.CAR-T cells injected alone on day 13 after tumor inoculation (without preadministration of NKG2D.ζ-NK cells) into mice bearing tumors devoid of MDSCs localized effectively to subcutaneous tumors in the flank (4 of 5 mice showed bioluminescent signal on days 14 and 18; FIG. 5C). There was a 10.5±0.8-fold increase in bioluminescent signal on day 18, with CAR-T cell bioluminescence remaining above baseline levels for the duration of the experiment (FIG. 5D). However, in tumors containing MDSCs, CAR-T cells localized poorly: only 1 of 5 mice exhibited bioluminescent signal (FIG. 5C), with only a 1.02±0.1-fold increase in bioluminescent signal on day 18 and bioluminescence falling below preinfusion levels within 10 days after injection (FIG. 5D). In contrast, preadministration of NKG2D.ζ-NK cells on day 10 into mice bearing MDSC-containing tumors allowed subsequently infused GD2.CAR-T cells to localize effectively to tumor sites, with bioluminescence in 5 of 5 mice at the tumor site and a 10.9±0.2-fold increase in bioluminescent signal on day 18, within 5 days of injection (FIG. 5D).

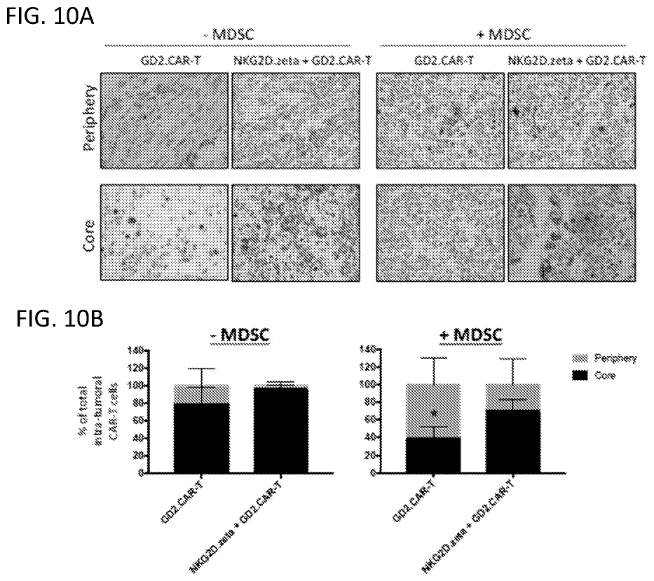

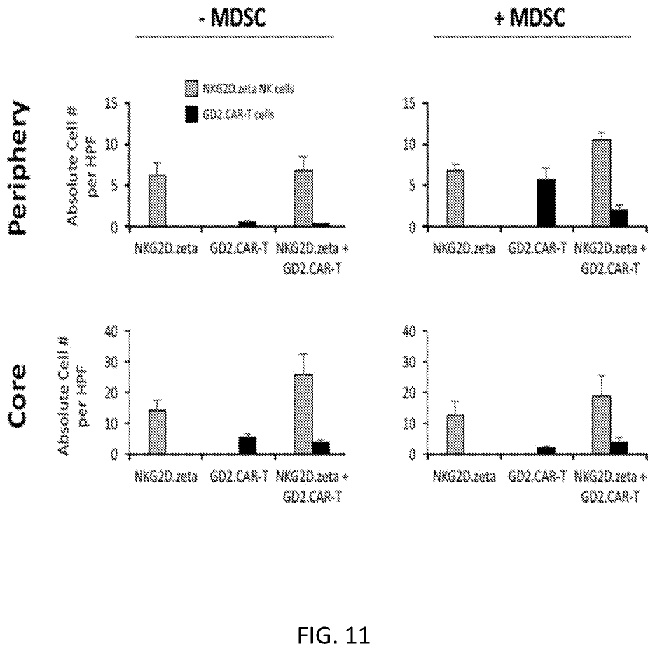

To determine if NKG2D.ζ-NK cells could promote GD2.CAR-T infiltration into the tumor bed, the inventors compared the frequency of human GD2.CAR-T and human NK cells in the tumor periphery and the tumor core by IHC (FIGS. 10A and 10B). In tumors without MDSCs, 89%±11% of the total T cells in the tumor had infiltrated into the tumor core. In contrast, a much smaller fraction (39%±16%) infiltrated into the core of tumors containing MDSCs, suggesting TME suppression of CAR-T infiltration. However, pretreatment of tumors containing MDSCs with NKG2D.ζ-NK cells increased the fraction of intratumoral CAR-T cells (70%±13%) within the tumor core. Equal numbers of NKG2D.ζ-NK cells were observed within both peripheral and core samples from MDSC-positive and MDSC-negative tumors (FIG. 11), suggesting the ability of NK cells to traffic well within tumors despite the presence of MDSCs.

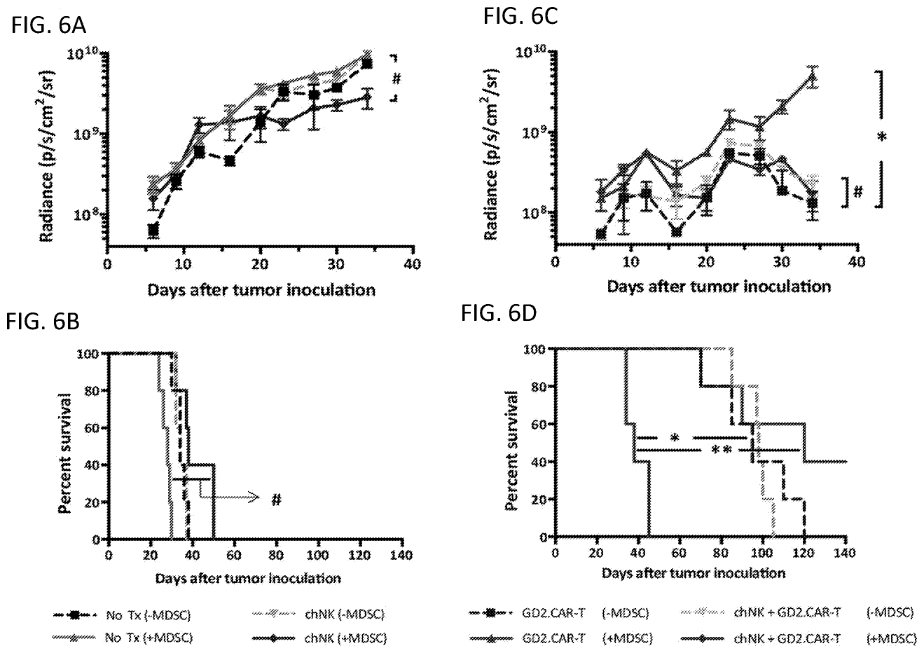

Elimination of MDSCs increases antitumor activity of GD2.CAR-T cells. To determine if the activities of NKG2D.ζ-NK cells described above enhance the antitumor function of CAR-T cells, the inventors treated mice bearing subcutaneous, luciferase-labeled neuroblastoma containing MDSCs with GD2.CAR-T cells preceded by NKG2D.ζ-NK cells, in a similar set-up to experiments in FIG. 5C. As seen in FIGS. 6A and 6B, a single injection of 5×10⁶ NKG2D.ζ-NK cells (a dose that achieved intratumoral MDSC depletion with only 26.8%±5.8% of the input MDSCs remaining) resulted in no significant tumor regression or prolongation of survival in mice bearing xenografts containing human MDSCs. A single infusion of 5×10⁶ GD2.CAR-T cells significantly reduced tumor in mice whose xenografts lacked human MDSCs with a median survival of 95 days (FIGS. 6C and 6D). However, the same GD2.CAR-T cells were ineffective against xenografts containing human MDSCs, worsening overall median survival to 39 days (FIG. 6B). In contrast, when the same GD2.CAR-T cell injection was preceded 3 days earlier by a single injection of 5×10⁶ NKG2D.ζ-NK cells (that had no direct antitumor effect by themselves within the other arm of the same experiment; see FIGS. 6A and 6B), the antitumor activity of the GD2.CAR-T cells in mice bearing MDSC-containing tumors was restored to the level observed in mice whose tumors lacked MDSCs (FIG. 6C). NKG2D.ζ-NK cells preinjection also improved the overall survival of the mice with MDSC-containing tumors to a median 120 days with durable cure in 2 of 5 mice (FIG. 6D). Taken together, the inventors' results suggest that NKG2D.ζ-NK cells not only eliminate MDSCs from the TME, but also recruit CAR-T cells to intratumoral sites, which facilitates antitumor efficacy.

Discussion. The inventors have developed a TME-disrupting approach that eliminates MDSCs and rescues MDSC-mediated impairment of tumor-directed CAR-T cells. The inventors show that when coimplanted with a neuroblastoma cell line, human MDSCs both enhance tumor growth and suppress the infiltration, expansion, and antitumor efficacy of tumor-specific CAR-T cells. In this model, NK cells bearing a chimeric version of the activating receptor NKG2D (NKG2D.ζ-NK cells) are directly cytotoxic to autologous MDSCs, thus eliminating MDSCs from tumors. In addition, NKG2D.ζ-NK cells secrete proinflammatory cytokines and chemokines in response to MDSCs at the tumor site, improving CAR-T cell infiltration and function, and resulting in tumor regression and prolonged survival compared with treatment with CAR-T cells alone. The inventors' cell therapy approach utilizes an engineered innate immune effector that targets the TME and shows potential to enhance efficacy of combination immune-based therapies for solid tumors.

NKG2D.ζ-NK cells directly killed highly suppressive MDSCs generated in vitro as well as those from patient tumors. NKG2D.ζ-NK cells also secreted cytokines that favored immune activation in response to MDSCs. Unmodified NK cells were unable to mediate these effects. The ability of NKG2D.ζ-NK cells to eliminate MDSCs from the TME should have several beneficial effects for antitumor immunity. First, as MDSCs express suppressive cytokines such as TGFβ and the checkpoint ligands PDL-1 and PDL-2, elimination of MDSCs should help relieve the suppression of endogenous T-cell responses and potentiate the activity of adoptive T-cell therapies. Given that high baseline numbers of MDSCs have been reported as a biomarker of poor response in the context of trials with the checkpoint inhibitors ipilimumab and pembrolizumab (35, 36), elimination of MDSCs by NKG2D.ζ-NK cells may also enhance checkpoint inhibition. Second, elimination of MDSCs should also decrease other MDSC-associated effects, including neovascularization via their expression of VEGF, production of immunosuppressive metabolic products such as PGE2 and adenosine, and establishment of tumor-supportive stroma via their expression of iNOS, FGF, and matrix metalloproteinases (8). In short, the ability of NKG2D.ζ-NK cells to eliminate MDSCs alters the TME in multiple ways that should improve antitumor immunity.

Previous strategies for modulation of MDSCs within the TME have included use of agents that block single functions such as secretion of nitric oxide (37) or expression of checkpoint molecules (38); induce MDSC differentiation such as with all-transretinoic acid (39); or eliminate MDSCs such as with the cytotoxic agents doxorubicin or cyclophosphamide (40). The MDSC-eliminating effects were dependent on continued administration of the agents, with a rapid rebound in MDSCs after discontinuation. Moreover, many of these agents have off-target toxicities that include damage to endogenous tumor-specific T cells. In contrast, NKG2D.ζ-NK cells produce prolonged and specific elimination of MDSCs with the potential to kill MDSCs that are recruited to the tumor from the bone marrow, while continually secreting cytokines and chemokines which, respectively, alter TME suppression and recruit and activate tumor-specific T cells. Thus, NKG2D.ζ-NK cells exert a prolonged combination of simultaneous immune-modulatory effects that enhance antitumor immune function in ways that could not be achieved by previous methods that target MDSCs.

The inventors observed no toxicity against normal hematopoietic cells when NKG2D.ζ was expressed in autologous human NK cells. Previous studies overexpressing an NKG2D.ζ receptor containing costimulatory endodomains (e.g., CD28 or 41BB) and DAP10, a signaling adaptor molecule for enhanced surface expression of NKG2D, in T cells showed activity against NKG2D ligand-overexpressing tumors, but at the cost of fratricide in vitro and lethal toxicity in mice (16, 32, 33). Using the inventors' standard T-cell activation/expansion protocol (22), the inventors also observed upregulation of NKG2D ligands, leading to fratricide in T cells expressing NKG2D.ζ. When NKG2D.ζ-T cells engage NKG2D ligands expressed on normal tissues, they will not receive the physiologic NK cell-directed inhibitory inputs that would safely regulate this potent and unopposed chimeric receptor activity. By contrast, when NKG2D.ζ is expressed on NK cells, they are able to recognize inhibitory NK cell ligands such as self-MHC expressed on healthy self-tissues, counteracting otherwise unopposed positive signals from NKG2D ligands. Thus, an NK cell platform for NKG2D enhancement may limit toxicity while taking advantage of the cytotoxic and immune-modulatory potential of the receptor-ligand system.

Unlike wild-type NKG2D, transgenic NKG2D.ζ expression and activity were not sensitive to downmodulation by TGFβ or soluble NKG2D ligands, allowing improved function in the TME. Native NKG2D relies solely on the intracytoplasmic adaptor DAP10 for mediating its cytolytic activity in human NK cells (41). TGFβ1 and soluble NKG2D ligands both decrease DAP10 gene transcription and protein activity, and thus reduce NKG2D function in endogenous NK cells (42, 43). In contrast, transgenic NKG2D.ζ does not rely on DAP10-based signaling for its activity, because signaling occurs through the ζ-chain. Thus, this construct provides a stable cytolytic pathway capable of circumventing TME-mediated downmodulation of native NKG2D activity. A previous study expressing a chimeric NKG2D.ζ molecule that incorporated DAP10 reported enhanced NK cytotoxicity compared with NKG2D.ζ alone in vitro against a variety of human cancer cell lines as well as in a xenograft model of osteosarcoma (44). However, this report did not address the susceptibility of this complex to downmodulation by TGFβ or soluble NKG2D ligands, or whether these NK cells had activity against MDSCs.

NKG2D.ζ-NK cells countered immunosuppression mediated by MDSCs leading to enhanced CAR-T cell tumor infiltration and expansion at tumor sites, CAR-T functions that are impaired in patients with solid tumors (45). Unlike the GD2.CAR-T cells in the inventors' model, NKG2D.ζ-NK cells homed effectively to MDSC-engrafted tumors and released an array of chemokines that increased T-cell infiltration of tumor. Unlike pharmacologic strategies aimed at enhancing leukocyte trafficking, including administration of lymphotactin or TNFα (46), the inventors' approach does not require continuous cytokine administration. In fact, the ability of chimeric NKG2D to augment NK immune function specifically within the immunosuppressive TME provides for the local release of chemotactic factors, reflecting a more homeostatic method by which to increase CAR-T infiltration. Once there, CAR-T cells should meet an environment favorably modified by NKG2D.ζ-NK cell-mediated elimination of MDSCs and production of proinflammatory cytokines. Indeed, elimination of MDSCs from a GD2+ tumor xenograft enhanced the activity of GD2.CAR-T cells in the inventors' model, including T-cell survival and intratumoral expansion. Given the suppressive effects of MDSCs in neuroblastoma (47, 48), the model shows how reversal of an MDSC-mediated suppressive microenvironment can improve antitumor functions of effector T cells.

Clinical neuroblastoma contains intense infiltrates of MDSCs (49), which are not included in tumor xenograft models currently used to study human cell therapeutics. The inventors' data suggest that coinoculation of tumors with suppressive components (such as MDSCs) can model TME-mediated suppression of CAR-T activity against solid tumors and provides a method by which to understand and counter immunosuppression. Although NSG mice lack a complete immune system in which to examine the effects of multiple endogenous immune components, the inventors' ability to engraft exogenous components (e.g., human MDSCs) within the inventors' TME model provides the possibility of simulating different immunosuppressive aspects of the solid TME. In fact, further model development utilizing human inhibitory macrophages and regulatory T cells (Treg) as additional suppressive components of the TME is performed.

In summary, the inventors describe an approach to reverse the suppressive TME using engineered human NK cells. The inventors have shown that generation and expansion of the inventors' NK cell product is feasible and that NKG2D.ζ-NK cells have antitumor activity within a suppressive solid TME without toxicity to normal NKG2D ligand-expressing tissues. Hence, the elimination of suppressive MDSCs by NKG2D.ζ-NK cells may safely enhance adoptive cellular immunotherapy for neuroblastoma and for many other tumors that are supported and protected by MDSCs.

### Example 2: Development of a Human NK Cell Immunotherapy Against Pediatric Sarcomas

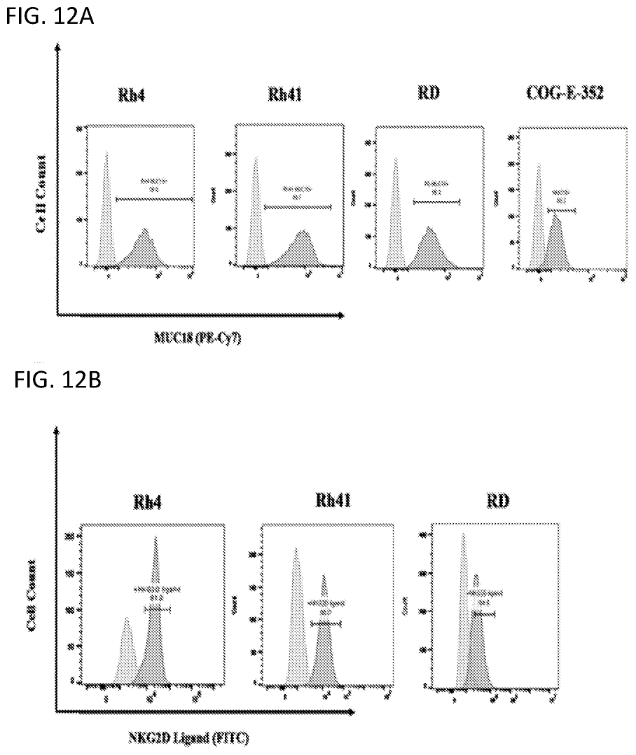

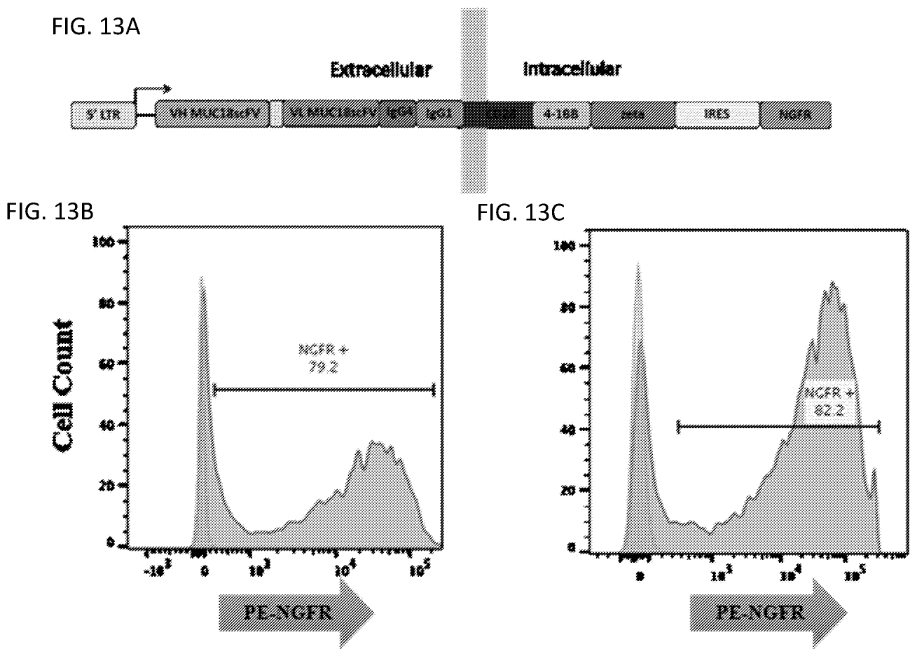

Screening of RMS cells lines (Rh4, Rh41, and RD) and a EWS line (COG-E-352) for expression of MUC18 and NKG2D ligands using flow cytometry confirmed the presence of both molecules (FIG. 12), thus verifying the potential for targeting of tumor cells and the TME via the inventors' approach. To initially demonstrate the function and specificity of a MUC18-targeting CAR molecule, a cytotoxic version of the CAR was first generated. The single chain variable fragment (scFv) sequence was obtained from a published anti-MUC18 monoclonal antibody (21). This initial cytotoxic construct contained a 41BB costimulatory endodomain and the cytotoxic z-chain of the T cell receptor complex (FIG. 13A). Human NK cells were transduced at high efficiencies with the CAR construct (FIG. 13B).

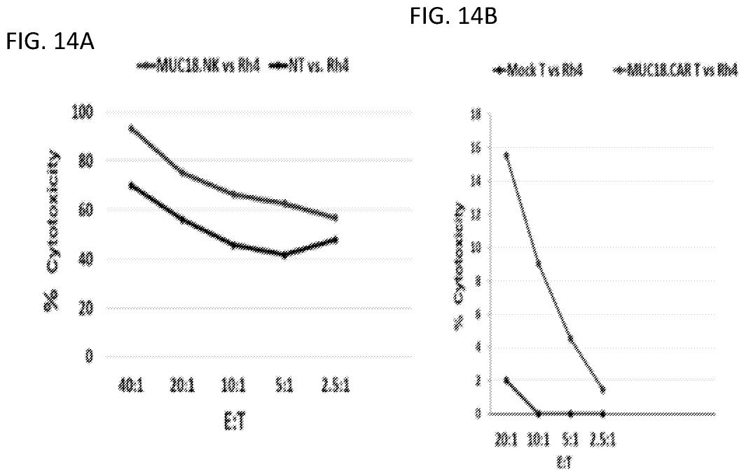

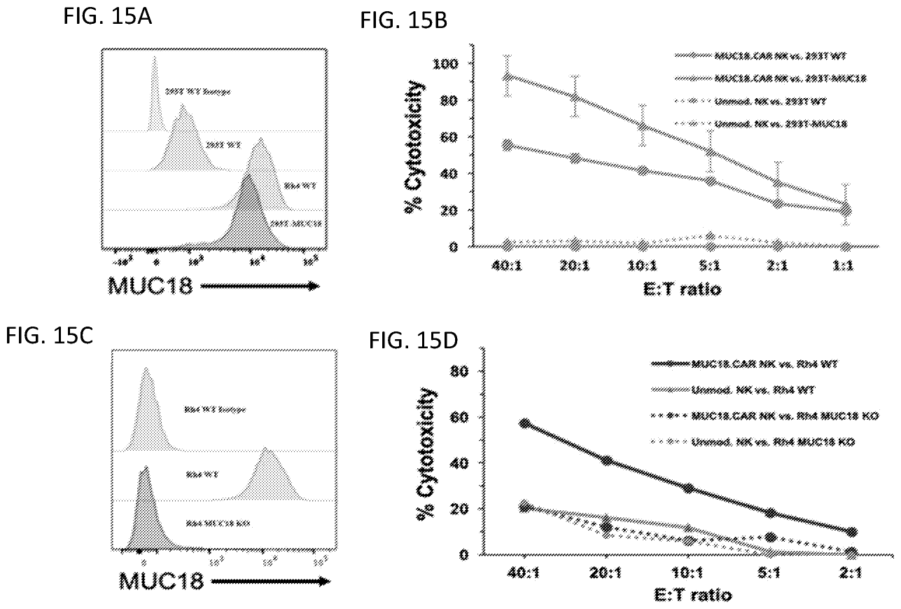

NK cells transduced with the MUC18.CAR were tested in a short-term cytotoxicity assay to assess their killing ability against Rh4, a primary alveolar RMS cell line. In chromium-release assays, MUC18.CAR NK cells exhibited higher cytotoxic activity against Rh4 than unmodified NK cells, with almost 100% killing at the 40:1 effector: target ratio (E:T) (FIG. 14A). Unmodified NK cells exhibited high baseline killing in this assay, reflecting activity from the native NKG2D receptor on non-transduced NK cells and lack of MHC Class I expression on Rh4. Of note, when the same MUC18.CAR was expressed on T cells, the MUC18.CAR T cells demonstrated highly specific, but significantly low, cytotoxic activity against Rh4 at all E:T ratios tested (FIG. 14B). In order to confirm the specificity of the MUC18.CAR, 293T cells were transfected with a MUC18 containing plasmid, significantly increasing the cell line's low baseline expression of MUC18 (FIG. 15A). Cytotoxic MUC18.CAR-transduced NK cells demonstrated high cytotoxicity against 293T-MUC18 transfected cells at all E:T ratios when compared to killing observed against 293T-WT cells (FIG. 15B). These findings confirmed the specificity of the CAR for the MUC18 target antigen, and suggested killing of targets bearing even low-levels of MUC18 by CAR-T cells, worrisome for toxicity. Finally, the inventors generated an Rh4 cell line using CRISPR-Cas9 technology genetically deficient for muc18 with complete abrogation of surface MUC18 expression (FIG. 15C) and performed short-term cytotoxicity assays with MUC18.CAR NK cells against Rh4-WT vs. Rh4-MUC18 KO cells. MUC18.CAR NK cells demonstrated cytotoxicity against Rh4 WT, while the MUC18.CAR NK cells and unmodified NK cells exhibited minimal killing of Rh4-MUC18 KO cells (FIG. 3D). Taken together, these experiments confirmed specificity and function of the MUC18 scFv-based CAR molecule.

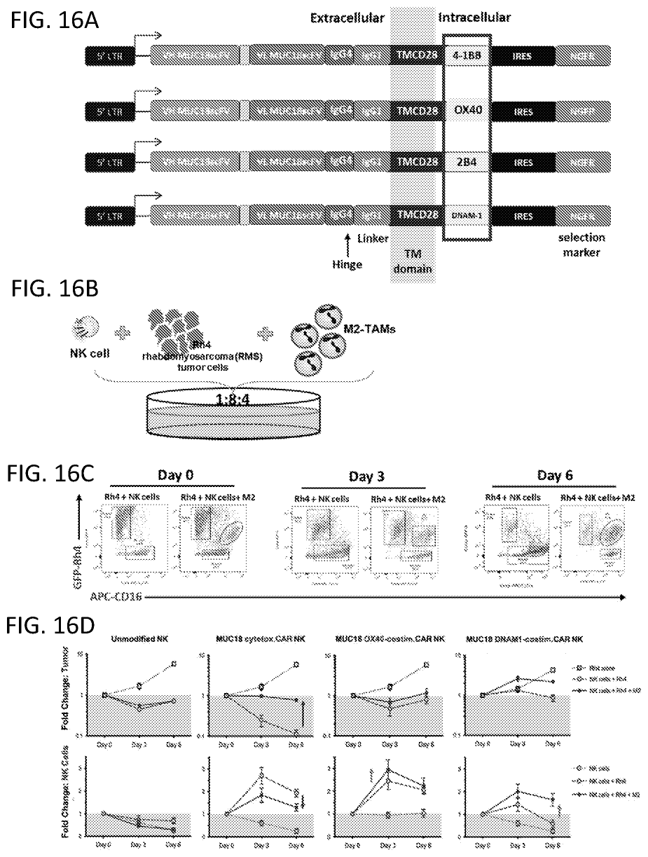

The inventors next generated, using in-Fusion cloning and PCR, MUC18 co-stim.CAR constructs (i.e., lacking a cytotoxic endodomain) with endodomains of the following costimulatory molecules: 41BB, OX40, 2B4, and DNAM-1 (FIG. 16A). To test the inventors' hypothesis that co-stim CAR-expressing NK cells would proliferate in the suppressive TME in presence of MUC18+ tumors and NKG2D ligand-expressing suppressive myeloid cells, the inventors developed a TME co-culture assay in which Rh4 tumors are cultured with NK cells in the presence of autologous inhibitory M2 macrophages (FIG. 16B). Co-culture assays in which tumors are cultured with NK cells in the presence of MDSCs and Tregs is also contemplated. Screening of NK cells transduced with all four MUC18 costim.CARs in this TME co-culture system revealed an enhanced ability of MUC18 co-stim.CAR NK cells expressing the OX40 and DNAM-1 endodomains to proliferate in the presence of tumor and inhibitory M2 macrophages compared to unmodified NK cells (FIG. 16C). Additional experiments will determine which construct results in optimal cytokine release and cytotoxicity of the modified NK cells. In addition, each construct that is co-transduced with NKG2D.ζ will be tested in ex vivo secondary NK cell expansion to assess for adequate survival and proliferation.

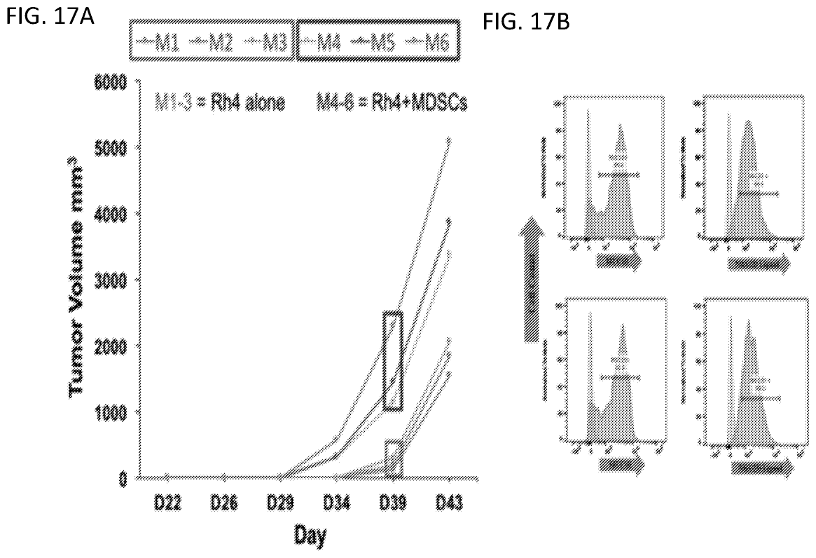

To develop an in vivo model that recapitulates the suppressive sarcoma TME, human ex vivo-enriched MDSCs were co-inoculated with Rh4 RMS tumor cells in NSG mice via subcutaneous flank injection and tumor growth kinetics were assessed (FIG. 17A). When tumors grew larger than euthanasia criteria, they were harvested, digested ex vivo, and flow cytometry was performed on tumor samples. As seen in FIG. 17B, tumor cells retained expression of MUC18 and NKG2D ligands during growth in the inventors' model. The inventors have also established a model in which one flank of an NSG mouse will contain sarcoma xenografts containing suppressive TME cells such as MDSCs, while the contralateral flank contains MUC18-expressing normal tissue to test safety.

To evaluate NK cell migration to tumors after infusion in the inventors' xenograft model, NK effector cells will be luciferase-labeled by retroviral transduction, and mice will be imaged every 5 days to assess NK cell migration. Expansion will be extrapolated by the intensity of signal at the tumor site. Tumor volume will be measured via calipers every 2-3 days to assess for tumor size.

Testing the safety of NK cells co-expressing a MUC18 co-stim.CAR and NKG2D.ζ against MUC18+ normal tissues. With the use of a normal tissue microarray (US Biomax), the inventors will perform immunohistochemistry with a MUC18 monoclonal antibody to assess MUC18 expression on numerous normal tissues, including vasculature, lung, heart, kidney, liver, muscle, and bone. To assess the safety of NK cells co-expressing the MUC18 co-stim.CAR with NKG2D.ζ, normal tissue shown to express MUC18 from the inventors' tissue microarray analysis will be plated as targets in short-term cytotoxicity assays with modified NK cells. In addition, long-term co-cultures will be performed and normal tissue and total NK cell numbers compared to baseline on day 0 will be enumerated. The inventors will compare killing of co-stim.CAR expressing NK cells with T cells to assess differential killing mediated by these cell platforms.

Determination of the ability of human NK cells co-expressing NKG2D.ζ and a MUC18 co-stim.CAR to produce anti-tumor responses while sparing normal tissue in two murine xenograft models of sarcoma. The following set of experiments will define an optimal co-stim.CAR on NK cells that meets the following criteria: MUC18 co-stim.CAR-modified NK cells co-expressing an NKG2D.ζ cytotoxicity receptor must (1.) exhibit enhanced proliferation in response to MUC18 tumor-associated antigen. (2.) mediate cytotoxicity against MUC18 and NKG2D ligand co-expressing tumors and NKG2D ligand-expressing MDSCs only in the TME, and (3.) should not activate killing of normal tissues that express MUC18 and self-MHC, but not NKG2D ligands. The inventors have developed two unique in vivo TME models that will allow testing NK cells bearing NKG2D.ζ and either the MUC18 OX40.co-stim or DNAM1.co-tim CAR: a TME xenograft described herein and a sarcoma PDX (7). These studies will define the co-stim CAR endodomain that will be cloned into a poly-cistronic vector (for ease of future NK transduction and product generation) to be utilized in the clinical trial.

Increasing doses of NK cells bearing NKG2D.ζ alone vs. MUC18 co-stim.CAR alone vs. NK cells bearing both chimeric molecules (n=10 per group) will be infused into mice bearing the inventors' novel TME xenograft as well as mice bearing sarcoma PDXs and the number of circulating and intra-tumoral MDSCs as well as tumor growth will be assessed.

An in vivo neuroblastoma model with a TME incorporating MDSCs, M2s, and Tregs has also been developed [21]. This model can be adapted with the Rh4 cell line and can be co-inject immunosuppressive cells of the TME, such as MDSCs (3×10⁵ MDSCs per 1×10⁶ tumor cells), at time of subcutaneous flank injection of tumor cells into NSG mice. After in vitro testing to determine the optimal MUC18 co-stim.CAR construct, in vivo testing of NK cells co-expressing NKG2D.ζ and MUC18 co-stim.CAR in NSG mice with Rh4 tumors and MDSCs will be performed. Control groups (with n=10 per group) will include NKG2D.ζ NK cells alone against Rh4 tumor alone, NKG2D.ζ NK cells alone against Rh4 tumor containing MDSCs, MUC18 co-stim CAR NK cells alone against Rh4 tumor alone, and MUC18 co-stim CAR NK cells alone against Rh4 tumor containing MDSCs. NK cell proliferation and activity against MDSCs will be correlated with a decrease in tumor growth.

To evaluate NK cell expansion at tumor sites after infusion in these xenograft models. NK effector cells will be luciferase-labeled by retroviral transduction (22) and mice will be imaged for NK bioluminescence every 3 days to assess NK cell localization to tumor. NK expansion will be extrapolated by increases in bioluminescent intensity at the tumor site. Tumor volume will be measured via calipers every 3 days to assess for treatment response. All experiments will also be done with T cells as the platform for NKG2D.ζ and MUC18 co-stim.CAR co-expression to contrast the activity and toxicity profile of T-vs. NK-cell platforms.

NK cells co-expressing NKG2D.ζ and MUC18 co-stim.CAR will mediate tumor regression in mice with MDSC-containing Rh4 tumors in a manner that is superior to the anti-tumor activity of NK cells bearing either construct alone. If NK cell expansion in vivo is poor, the treatment protocol will be adjusted to include cytokine co-stimulation such as constitutively active IL-15 transgene (23) or constitutively active IL-7 receptor endodomain (24).

T cells expressing a MUC18 cytotoxic CAR or co-stim.CAR will mediate toxicity against normal tissue, reflecting the potential for toxicity with the T-cell platform. In contrast, NK cells expressing a MUC18 co-stim.CAR will only proliferate in response to normal tissue, but not mediate any killing. If killing by NK cells with some of the co-stim constructs is observed, these constructs will be screened out as potentially toxic.

Development and validation of GMP-compliant manufacturing processes for the production of co-modified human NK cells for the treatment of patients with relapsed/refractory RMS and EWS. The inventors will develop standard operating procedures (SOPs) for GMP-compliant retroviral transduction (using transient supernatants (25), instead of virus-producing cell line validation) and expansion of human NK cells. The inventors have published on optimal clinical-grade expansion and genetic modification of human NK cells and thus have some SOPs templates already in place (26). The inventors will perform n=5 normal donor test-runs to validate the SOPs and ensure adequacy of NK cell numbers for indicated treatment doses.

### Example 3: Exemplary Methods and Materials

Cytokines, cell lines, and antibodies. Recombinant human interleukin (IL)2 was obtained from the National Cancer Institute Biological Resources Branch (Frederick, MD). Recombinant human IL6, GM-CSF, IL7, and IL15 were purchased from PeproTech. The human neuroblastoma cell line LAN-1 was purchased from ATCC and cultured in DMEM culture medium supplemented with 2 mmol/L L-glutamine (Gibco-BRL) and 10% FBS (HyClone). The human CML cell line K562 was purchased from ATCC and cultured in complete-RPMI culture medium composed of RPMI-1640 medium (HyClone) supplemented with 2 mmol/L L-glutamine and 10% FBS. A modified version of parental K562 cells, genetically modified to express a membrane-bound version of IL15 and 41BB ligand, K562-mb15-41BB-L, was kindly provided by Dr. Dario Campana (National University of Singapore). All cell lines were verified by either genetic or flow cytometry-based methods (LAN-1 and K562 authenticated by ATCC in 2009) and tested for Mycoplasma contamination monthly via MycoAlert (Lonza) mycoplasma enzyme detection kit (last mycoplasma testing of LAN-1, K562 parental line, and K562-mb15-41BB-L on Nov. 2, 2018; all negative). All cell lines were used within 1 month of thawing from early-passage (<3 passages of original vial) lots.

CAR-encoding retroviral vectors. The construction of the SFG-retroviral vector encoding GD2-CAR.41BB.ζ, as shown in FIG. 7A, was previously described (17). The SFG-retroviral vector encoding NKG2D.ζ, an internal ribosomal entry site (IRES), and truncated CD19 (tCD19), was generated by subcloning NKG2D.ζ from a retroviral vector (18) kindly provided by Dr. Charles L. Sentman (Dartmouth Geisel School of Medicine, Hanover, NH) into pSFG.IRES.tCD19 (19). RD114-speudotyped viral particles were produced by transient transfection in 293T cells, as previously described (20).

Expansion and retroviral transduction of human NK and T cells. Human NK cells were activated, transduced with retroviral constructs (FIG. 1A), and expanded as previously described by the inventors' laboratory (21). Briefly, peripheral blood mononuclear cells (PBMC), obtained from healthy donors under Baylor College of Medicine IRB-approved protocols, were cocultured with irradiated (100 Gy) K562-mb15-41BB-L at a 1:10 (NK cell:irradiated tumor cell) ratio in G-Rex cell culture devices (Wilson Wolf) for 4 days in Stem Cell Growth Medium (CellGenix) supplemented with 10% FBS and 500 IU/mL IL2. Cell suspensions on day 4 (containing 50%-70% expanded/activated NK cells) were transduced with SFG-based retroviral vectors, as previously described (22). The transduced cell population was then subjected to secondary expansion to generate adequate cell numbers for experiments in G-Rex devices at the same NK cell:irradiated tumor cell ratio with 100 IU/mL IL2. This 17-day human gene-modified NK cell protocol resulted in >97% pure CD56⁺/CD3⁻ NK cell population with average 77.4%±18.2% (n=25) of NK cells transduced with the construct of interest. For most experiments, transduced NK cells were purified to >95% by magnetic column selection of truncated CD19 selection marker-positive cells.

For production of GD2.CAR-T cells (autologous to MDSCs and NK cells), PBMCs from healthy donors were suspended in T-cell medium (TCM) consisting of RPMI-1640 supplemented with 45% Click's Medium (Gibco-BRL), 10% FBS, and 2 mmol/L L-glutamine, and cultured in wells precoated with CD3 (OKT3, CRL-8001; ATCC) and CD28 (clone CD28.2; BD Biosciences) antibodies for activation. Human IL15 and IL7 were added on day +1, and cells underwent retroviral transduction on day +2, as previously described (22). T cells were used for experiments between days +9 to +14 posttransduction, with phenotype as shown in Supplementary FIGS. 7B and 7C.

Induction and enrichment of human MDSCs. The inventors' method for ex vivo generation of human PBMC-derived MDSCs was derived from published reports (23), with slight modifications. Briefly, PBMCs were sequentially depleted of CD25^{hi}-expressing cells and CD3-expressing cells by magnetic column separation (Miltenyi Biotec). Resultant CD25^{lo/-}, CD3-PBMCs were plated at 4×10⁶ cells/mL in complete-RPMI medium with human IL6 and GM-CSF (both at 20 ng/mL) onto 12-well culture plates (Sigma Corning) at 1 mL/well. Plates were incubated for 7 days with medium and cytokines being replenished on days 3 and 5. Resultant cells were harvested by gentle scraping, and MDSCs were purified by magnetic selection using CD33 magnetic microbeads (Miltenyi Biotec). Cells were analyzed by multicolor flow cytometry for CD33, CD14, CD15, HLA-DR. CD11b, CD83, and CD163 (BD Biosciences). MDSCs were defined as either monocytic (M-MDSCs; CD14+, HLA-DR^{low/-}), PMN-MDSCs (CD14⁻, CD15⁺, CD11b⁺), or early-stage MDSCs (lineage⁻, HLA-DR^{low/-}, CD33⁺), as per published guidelines (24). In addition to the above markers, MDSCs were stained for PD-L1, PD-L2, and NKG2D ligands via an NKG2D-Fc chimera (BD Biosciences) followed by FITC-labeled anti-Fc. This pan-ligand staining approach was determined to be the most efficient way to assess NKG2D ligand expression on human MDSCs because (i) NKG2D ligand expression had not previously been reported for human MDSCs and thus simultaneous evaluation of the eight different NKG2D ligands would have been required, and (ii) the inventors found poor reproducibility in staining patterns using individual commercially available ligand antibodies, even within the same donor.

In vitro T-cell suppression assay. T-cell proliferation was assessed using CellTrace Violet (Thermo Fisher) dye dilution analysis, as per manufacturer's recommendations. Briefly, 1× 10⁵ CellTrace Violet-labeled T cells (isolated at the time of MDSC generation) were plated onto 96-well plates in the presence of plate-bound 1 μg/mL CD3 and 1 μg/mL CD28 antibodies with 50 IU/mL IL2 in the absence or presence of autologous MDSCs or peripheral blood monocytes (as a myeloid cell control) at 1:1, 4:1, and 8:1 T-cell:MDSC ratios. In some experiments, only the 4:1 ratio is shown as this was determined as optimal for assessment of suppression. After 4 days of coculture, T cells were labeled with CD3 antibody and assessed for cell division using CellTrace Violet dye dilution by flow cytometry. Percent suppression was calculated as follows: [(% proliferating T cells in the absence of MDSCs-% proliferating T cells in presence of MDSCs)/% proliferating T cells in the absence of MDSCs]×100. Proliferation was defined as a percentage of T cells undergoing active division as represented by CellTrace Violet dilution peaks, as previously described (25).

In vitro CAR-T chemotaxis assay. Transwell 5-μm pore inserts (Corning) for migration experiments were prepared by coating with 0.01% gelatin at 37° C. overnight, followed by 3 μg of human fibronectin (Life Technologies) at 37° C. for 3 hours to mimic endothelial and extracellular matrix components, as previously described (26). Briefly, 2× 10⁵ purified GD2.CAR-T cells were placed in 100 μL of TCM in the upper chambers of the precoated Transwell inserts that were then transferred into wells of a 24-well plate. Culture supernatants (400 μL), from NKG2D.ζ or unmodified NK cells cultured with autologous MDSCs or monocytes, were placed in the lower chambers of the wells. Plain medium or medium supplemented with 1 μg/mL of the T-cell recruiting chemokine, MIG, served as negative and positive controls, respectively. The plates were then incubated for 4 hours at 37° C. with 5% CO₂, followed by a 10-minute incubation at 4° C. to loosen any cells adhering to the undersides of the insert membranes. The fluid in the lower chambers was collected separately, and migrated cells were counted using trypan blue exclusion. The cells were analyzed for CAR expression by flow cytometry to confirm phenotype of migrated T cells.

In vivo TME model. Twelve- to 16-week-old female NSG mice were implanted subcutaneously in the dorsal right flank with 1×10⁶ Firefly luciferase (FfLuc)-expressing LAN-1 neuroblastoma cells admixed with 3×10⁵ ex vivo-generated MDSCs, suspended in 100 μL of basement membrane Matrigel (Corning). Matrigel basement membrane was important in keeping tumor and MDSCs confined so as to establish a localized solid TME. Ten to 14 days later, when tumors measured at least 100 mm³ by caliper measurement, mice were injected intravenously with 5×10⁶ GD2.CAR-T cells. Tumor growth was measured twice weekly by live bioluminescence imaging using the IVIS system (IVIS, Xenogen Corporation) 10 minutes after 150 mg/kg D-luciferin (Xenogen)/mouse was injected intraperitoneally. In experiments examining the ability of NKG2D.ζ-NK cells to reduce intratumoral MDSCs, 1×10⁷ unmodified or NKG2D.ζ-NK cells were injected intravenously when tumors measured at least 100 mm3. At the end of the experiment, tumors were harvested en bloc, digested ex vivo, and intratumoral human MDSCs (CD33⁺, HLA-DR^{low} cells) were enumerated by flow cytometry. The absolute number of human MDSCs within a tumor digest was enumerated per mouse (n=5 mice/group), compared with pretreatment MDSC numbers, and presented as mean % MDSCs remaining per treatment group. In experiments examining the effects of NKG2D.ζ-NK cells on GD2.CAR-T cell antitumor activity, 5×10⁶ (cell dose chosen to mitigate direct antitumor effects of NK cells) unmodified or NKG2D.ζ-NK cells were injected intravenously 3 days prior to GD2.CAR-T injection. In GD2.CAR-T cell homing experiments, CAR-T were transduced with GFP-luciferase retroviral construct prior to injection into mice bearing unmodified tumor cells (27). Mice received 5,000 IU human IL2 intraperitoneally three times per week for 3 weeks following NK cell injection to promote NK cell survival in NSG mice (28). Tumor size was measured twice weekly with calipers, and the mice were imaged for bioluminescence signal from T cells at the same time. Mice were euthanized for excessive tumor burden, as per protocol guidelines. The animal studies protocol was approved by Baylor College of Medicine Institutional Animal Care and Use Committee, and mice were treated in strict accordance with the institutional guidelines for animal care.

IHC of neuroblastoma xenografts. On day 32 of in vivo experiments, animals were sacrificed, tumors were harvested, and sectioned bluntly ex vivo to separate tumor periphery (outer ⅓ of tumor volume) versus core (nonnecrotic inner ⅔ of tumor volume), and n=5 sections/tumor sample were analyzed for the presence of GD2.CAR-T and NKG2D.ζ-NK cells by H&E and human CD3 and CD57 immunostaining performed by the Human Tissue Acquisition and Pathology Core of Baylor College of Medicine. Lack of CD57 expression on infused GD2.CAR-T was confirmed by flow cytometry prior to administration. CD57 was chosen as the marker for NK cells in tumor tissue in the inventors' study because LAN-1 tumors naturally express the prototypical NK marker CD56, truncated CD19 expression was inadequate for in situ staining, and CD57 had previously been used as a marker for tissue-localized activated NK cells (28). The number of human CD3+ and CD57+ cells in representative sections of tumors from periphery versus core of the treatment groups indicated were enumerated per high-powered field at 40× magnification, and the percentage of the total number of cells enumerated within tumors found in the periphery versus core in each treatment group indicated from tumors with and without MDSCs is shown as mean±SEM of n=5 sections/periphery or core, n=5 tumors/group.

Analysis of intratumoral MDSCs from patients with neuroblastoma. Tumor tissue and matched peripheral blood of neuroblastoma patients obtained in the context of a specimen/laboratory study after patient identification had been removed were thawed and analyzed for MDSC subsets by flow cytometry or utilized in in vitro assays, as described in figure legends or Results. The tissue acquisition protocol was performed after review and approval by the Baylor College of Medicine Institutional Review Board. Briefly, subjects with a diagnosis of high-risk or intermediate-risk neuroblastoma were eligible to participate. Written informed consent, or appropriate assent for participation, in accordance with the Declaration of Helsinki was obtained from each subject or subject's guardian for procurement of patient blood and tumor tissue and for subsequent analyses of stored patient materials.

Statistical analysis. Data are presented as mean±SEM of either experimental replicates or number of donors, as indicated. A paired two-tailed t test was used to determine significance of differences between means, with P<0.05 indicating a significant difference. For in vivo bioluminescence, changes in tumor radiance from baseline at each time point were calculated and compared between groups using a two-sample t test. Multiple group comparisons were conducted via ANOVA via GraphPad Prism v7 software. Survival determined from the time of tumor cell injection was analyzed by Kaplan-Meier and differences in survival between groups were compared by the log-rank test.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
U.S. Pat. No. 9,447,194
U.S. Pat. No. 4,554,101
U.S. Pat. No. 8,735,553
U.S. Pat. No. 8,354,509
U.S. Pat. No. 6,207,156
U.S. Pat. No. 8,119,129
U.S. Pat. No. 8,017,114
U.S. Patent Application No. US2003/0022367
U.S. Patent Application No. US2014/0294898
U.S. Patent Application No. US2014/022021
U.S. Patent Application No. US2011/0008369
International Patent Application No. WO2001/014424
International Patent Application No. WO2000/037504
International Patent Application No. WO2001/14424
International Patent Application No. WO1998/42752
International Patent Application No. WO2000/37504

### REFERENCE SET 1

1. Fesnak A D, June C H, Levine B L. Engineered T cells: the promise and challenges of cancer immunotherapy. Nat Rev Cancer 2016; 16:566-81.CrossRef
2. Maude S L, Frey N, Shaw P A, Aplenc R, Barrett D M, Bunin N J, et al. Chimeric antigen receptor T cells for sustained remissions in leukemia. N Engl J Med 2014; 371:1507-17.
3. Davila M L, Riviere I, Wang X, Bartido S, Park J, Curran K, et al. Efficacy and toxicity management of 19-28z CAR T cell therapy in B cell acute lymphoblastic leukemia. Sci Transl Med 2014; 6:224ra25.
4. Hege K M, Bergsland E K, Fisher G A, Nemunaitis J J, Warren R S, McArthur J G, et al. Safety, tumor trafficking and immunogenicity of chimeric antigen receptor (CAR)-T cells specific for TAG-72 in colorectal cancer. J Immunother Cancer 2017; 5:22.
5. Thistlethwaite F C, Gilham D E, Guest R D, Rothwell D G, Pillai M, Burt D J, et al. The clinical efficacy of first-generation carcinoembryonic antigen (CEACAM5)-specific CAR T cells is limited by poor persistence and transient pre-conditioning-dependent respiratory toxicity. Cancer Immunol Immunother 2017; 66:1425-36.
6. Burga R A, Thorn M, Point G R, Guha P, Nguyen C T, Licata L A, et al. Liver myeloid-derived suppressor cells expand in response to liver metastases in mice and inhibit the anti-tumor efficacy of anti-CEA CAR-T. Cancer Immunol Immunother 2015; 64:817-29.
7. Gabrilovich D I, Nagaraj S. Myeloid-derived suppressor cells as regulators of the immune system. Nat Rev Immunol 2009; 9:162-74.
8. Marvel D, Gabrilovich D I. Myeloid-derived suppressor cells in the tumor microenvironment: expect the unexpected. J Clin Invest 2015; 125:3356-64.
9. Diaz-Montero C M, Salem M L, Nishimura M I, Garrett-Mayer E, Cole D J, Montero A J. Increased circulating myeloid-derived suppressor cells correlate with clinical cancer stage, metastatic tumor burden, and doxorubicin-cyclophosphamide chemotherapy. Cancer Immunol Immunother 2009; 58:49-59.
10. Caligiuri M A. Human natural killer cells. Blood 2008; 112:461-9.
11. Ferlazzo G, Morandi B. Cross-talks between natural killer cells and distinct subsets of dendritic cells. Front Immunol 2014; 5:159.
12. Stojanovic A, Cerwenka A. Natural killer cells and solid tumors. J Innate Immun 2011; 3:355-64.
13. Gonzalez S, Lopez-Soto A, Suarez-Alvarez B, Lopez-Vazquez A, Lopez-Larrea C. NKG2D ligands: key targets of the immune response. Trends Immunol 2008; 29:397-403.
14. Raffaghello L, Prigione I, Airoldi I, Camoriano M, Levreri I, Gambini C, et al. Downregulation and/or release of NKG2D ligands as immune evasion strategy of human neuroblastoma. Neoplasia 2004; 6:558-68.
15. Dasgupta S, Bhattacharya-Chatterjee M, O'Malley B W Jr., Chatterjee S K. Inhibition of N K cell activity through TGF-beta 1 by down-regulation of NKG2D in a murine model of head and neck cancer. J Immunol 2005; 175:5541-50.
16. Barber A, Rynda A, Sentman C L. Chimeric NKG2D expressing T cells eliminate immunosuppression and activate immunity within the ovarian tumor microenvironment. J Immunol 2009; 183:6939-47.
17. Heczey A, Liu D, Tian G, Courtney A N, Wei J, Marinova E, et al. Invariant NKT cells with chimeric antigen receptor provide a novel platform for safe and effective cancer immunotherapy. Blood 2014; 124:2824-33.
18. Zhang T, Barber A, Sentman C L. Generation of antitumor responses by genetic modification of primary human T cells with a chimeric NKG2D receptor. Cancer Res 2006; 66:5927-33.
19. Shaffer D R, Savoldo B, Yi Z, Chow K K, Kakarla S, Spencer D M, et al. T cells redirected against CD70 for the immunotherapy of CD70-positive malignancies. Blood 2011; 117:4304-14.
20. Kelly P F, Carrington J, Nathwani A, Vanin E F. RD114-pseudotyped oncoretroviral vectors. Biological and physical properties. Ann N Y Acad Sci 2001; 938:262-76; discussion 76-7.
21. Lapteva N, Durett A G, Sun J, Rollins L A, Huye L L, Fang J, et al. Large-scale ex vivo expansion and characterization of natural killer cells for clinical applications. Cytotherapy 2012; 14:1131-43.
22. Xu Y, Zhang M, Ramos C A, Durett A, Liu E, Dakhova O, et al. Closely related T-memory stem cells correlate with in vivo expansion of CAR.CD19-T cells and are preserved by IL-7 and IL-15. Blood 2014; 123:3750-9.
23. Lechner M G, Liebertz D J, Epstein A L. Characterization of cytokine-induced myeloid-derived suppressor cells from normal human peripheral blood mononuclear cells. J Immunol 2010; 185:2273-84.
24. Bronte V, Brandau S, Chen S H, Colombo M P, Frey A B, Greten T F, et al. Recommendations for myeloid-derived suppressor cell nomenclature and characterization standards. Nat Commun 2016; 7:12150.
25. Kruisbeek A M, Shevach E, Thornton A M. Proliferative assays for T cell function. Curr Protoc Immunol 2004; Chapter 3: Unit 3 12.
26. Roda J M, Parihar R, Magro C, Nuovo G J, Tridandapani S, Carson W E. Natural killer cells produce T cell-recruiting chemokines in response to antibody-coated tumor cells. Cancer Res 2006; 66:517-26.
27. Charo J, Perez C, Buschow C, Jukica A, Czeh M, Blankenstein T. Visualizing the dynamic of adoptively transferred T cells during the rejection of large established tumors. Eur J Immunol 2011; 41:3187-97.
28. Miller J S, Rooney C M, Curtsinger J, McElmurry R, McCullar V, Verneris M R, et al. Expansion and homing of adoptively transferred human natural killer cells in immunodeficient mice varies with product preparation and in vivo cytokine administration: implications for clinical therapy. Biol Blood Marrow Transplant 2014; 20:1252-7.
29. Groh V, Wu J, Yee C, Spies T. Tumour-derived soluble MIC ligands impair expression of NKG2D and T-cell activation. Nature 2002; 419:734-8.
30. Markowitz J, Wesolowski R, Papenfuss T, Brooks T R, Carson W E. Myeloid-derived suppressor cells in breast cancer. Breast Cancer Res Treat 2013; 140:13-21.
31. Xue Q, Bettini E, Paczkowski P, Ng C, Kaiser A, McConnell T, et al. Single-cell multiplexed cytokine profiling of CD19 CAR-T cells reveals a diverse landscape of polyfunctional antigen-specific response. J Immunother Cancer 2017; 5:85.
32. Spear P, Barber A, Rynda-Apple A, Sentman C L. Chimeric antigen receptor T cells shape myeloid cell function within the tumor microenvironment through IFN-γ and G M-CSF. J Immunol 2012; 188:6389-98.
33. VanSeggelen H, Hammill J A, Dvorkin-Gheva A, Tantalo D G, Kwiecien J M, Denisova G F, et al. T cells engineered with chimeric antigen receptors targeting NKG2D ligands display lethal toxicity in mice. Mol Ther 2015; 23:1600-10.
34. Qu P, Wang L Z, Lin P C. Expansion and functions of myeloid-derived suppressor cells in the tumor microenvironment. Cancer Lett 2016; 380:253-6.
35. Martens A, Wistuba-Hamprecht K, Geukes Foppen M, Yuan J, Postow M A, Wong P, et al. Baseline peripheral blood biomarkers associated with clinical outcome of advanced melanoma patients treated with ipilimumab. Clin Cancer Res 2016; 22:2908-18.
36. Weide B, Martens A, Hassel J C, Berking C, Postow M A, Bisschop K, et al. Baseline biomarkers for outcome of melanoma patients treated with pembrolizumab. Clin Cancer Res 2016; 22:5487-96.
37. Jayaraman P, Parikh F, Lopez-Rivera E, Hailemichael Y, Clark A, Ma G, et al. Tumor-expressed inducible nitric oxide synthase controls induction of functional myeloid-derived suppressor cells through modulation of vascular endothelial growth factor release. J Immunol 2012; 188:5365-76.
38. Serafini P, Meckel K, Kelso M, Noonan K, Califano J, Koch W, et al. Phosphodiesterase-5 inhibition augments endogenous antitumor immunity by reducing myeloid-derived suppressor cell function. J Exp Med 2006; 203:2691-702.
39. Mirza N, Fishman M, Fricke I, Dunn M, Neuger A M, Frost T J, et al. All-trans-retinoic acid improves differentiation of myeloid cells and immune response in cancer patients. Cancer Res 2006; 66:9299-307.
40. Alizadeh D, Katsanis E, Larmonier N. Chemotherapeutic targeting of myeloid-derived suppressor cells. Oncoimmunology 2014; 3:e27359.
41. Roda-Navarro P, Reyburn H T. The traffic of the NKG2D/Dap10 receptor complex during natural killer (NK) cell activation. J Biol Chem 2009; 284:16463-72.
42. Park Y P, Choi S C, Kiesler P, Gil-Krzewska A, Borrego F, Weck J, et al. Complex regulation of human NKG2D-DAP10 cell surface expression: opposing roles of the gammac cytokines and TGF-beta1. Blood 2011; 118:3019-27.
43. Coudert J D, Zimmer J, Tomasello E, Cebecauer M, Colonna M, Vivier E, et al. Altered NKG2D function in NK cells induced by chronic exposure to NKG2D ligand-expressing tumor cells. Blood 2005; 106:1711-7.
44. Chang Y H, Connolly J, Shimasaki N, Mimura K, Kono K, Campana D. A chimeric receptor with NKG2D specificity enhances natural killer cell activation and killing of tumor cells. Cancer Res 2013; 73:1777-86.
45. Moon E K, Wang L C, Dolfi D V, Wilson C B, Ranganathan R, Sun J, et al. Multifactorial T-cell hypofunction that is reversible can limit the efficacy of chimeric antigen receptor-transduced human T cells in solid tumors. Clin Cancer Res 2014; 20:4262-73.
46. Cairns C M, Gordon J R, Li F, Baca-Estrada M E, Moyana T, Xiang J. Lymphotactin expression by engineered myeloma cells drives tumor regression: mediation by CD4+ and CD8+ T cells and neutrophils expressing XCR1 receptor. J Immunol 2001; 167:57-65.
47. Gowda M, Payne K K, Godder K, Manjili M H. HLA-D R expression on myeloid cells is a potential prognostic factor in patients with high-risk neuroblastoma. Oncoimmunology 2013; 2:e26616.
48. Pistoia V, Morandi F, Bianchi G, Pezzolo A, Prigione I, Raffaghello L. Immunosuppressive microenvironment in neuroblastoma. Front Oncol 2013; 3:167.
49. Heczey A, Louis C U, Savoldo B, Dakhova O, Durett A, Grilley B, et al. CAR T cells administered in combination with lymphodepletion and PD-1 inhibition to patients with neuroblastoma. Mol Ther 2017; 25:2214-24.

### REFERENCE SET 2

1. Hettmer S, Li Z, Billin A N, Barr F G, Cornelison D D, Ehrlich A R, Guttridge D C, Hayes-Jordan A, Helman L J, Houghton P J, et al. Rhabdomyosarcoma: current challenges and their implications for developing therapies. Cold Spring Harb Perspect Med. 2014; 4(11):a025650.
2. Mascarenhas L, Lyden E R, Breitfeld P P, Walterhouse D O, Donaldson S S, Rodeberg D A, Parham D M, Anderson J R, Meyer W H, and Hawkins D S. Risk-based treatment for patients with first relapse or progression of rhabdomyosarcoma: A report from the Children's Oncology Group. Cancer. 2019; 125(15):2602-9.
3. DeRenzo C, Krenciute G, and Gottschalk S. The Landscape of CAR T Cells Beyond Acute Lymphoblastic Leukemia for Pediatric Solid Tumors. Am Soc Clin Oncol Educ Book. 2018; 38(830-7.
4. Barber A, Rynda A, and Sentman C L. Chimeric NKG2D expressing T cells eliminate immunosuppression and activate immunity within the ovarian tumor microenvironment. J Immunol. 2009; 183(11):6939-47.
5. Parihar R, Rivas C, Huynh M, Omer B, Lapteva N, Metelitsa L S, Gottschalk S M, and Rooney C M. NK Cells Expressing a Chimeric Activating Receptor Eliminate MDSCs and Rescue Impaired CAR-T Cell Activity against Solid Tumors. Cancer Immunol Res. 2019; 7(3):363-75.
6. Orentas R J, Yang J J, Wen X, Wei J S, Mackall C L, and Khan J. Identification of cell surface proteins as potential immunotherapy targets in 12 pediatric cancers. Frontiers in oncology. 2012; 2(194.
7. Rainusso N, Cleveland H, Hernandez J A, Quintanilla N M, Hicks J, Vasudevan S, Marco R A W, Allen-Rhoades W, Wang L L, and Yustein J T. Generation of patient-derived tumor xenografts from percutaneous tumor biopsies in children with bone sarcomas. Pediatric blood & cancer. 2019; 66(4):e27579.
8. Wu J, and Lanier L L. Natural killer cells and cancer. Advances in cancer research. 2003; 90(127-56.
9. Lanier L L. Up on the tightrope: natural killer cell activation and inhibition. Nat Immunol. 2008; 9(5):495-502.
10. Caligiuri M A. Human natural killer cells. Blood. 2008; 112(3):461-9.
11. Farag S S, Bacigalupo A, Eapen M, Hurley C, Dupont B, Caligiuri M A, Boudreau C, Nelson G, Oudshoorn M, van Rood J, et al. The effect of KIR ligand incompatibility on the outcome of unrelated donor transplantation: a report from the center for international blood and marrow transplant research, the European blood and marrow transplant registry, and the Dutch registry. Biology of blood and marrow transplantation: journal of the American Society for Blood and Marrow Transplantation. 2006; 12(8):876-84.
12. Cooper M A, Fehniger T A, Turner S C, Chen K S, Ghaheri B A, Ghayur T, Carson W E, and Caligiuri M A. Human natural killer cells: a unique innate immunoregulatory role for the CD56(bright) subset. Blood. 2001; 97(10):3146-51.
13. Melnikova V O, and Bar-Eli M. Bioimmunotherapy for melanoma using fully human antibodies targeting MCAM/MUC18 and IL-8. Pigment Cell Res. 2006; 19(5):395-405.
14. Yuan Y. Spatial Heterogeneity in the Tumor Microenvironment. Cold Spring Harbor perspectives in medicine. 2016; 6(8):a026583.
15. Binnewies M, Roberts E W, Kersten K, Chan V, Fearon D F, Merad M, Coussens L M, Gabrilovich D I, Ostrand-Rosenberg S, Hedrick C C, et al. Understanding the tumor immune microenvironment (TIME) for effective therapy. Nature medicine. 2018; 24(5):541-50.
16. Gabrilovich D I, and Nagaraj S. Myeloid-derived suppressor cells as regulators of the immune system. Nature reviews Immunology. 2009; 9(3):162-74.
17. Martens A, Wistuba-Hamprecht K, Geukes Foppen M, Yuan J, Postow M A, Wong P, Romano E, Khammari A, Dreno B, Capone M, et al. Baseline Peripheral Blood Biomarkers Associated with Clinical Outcome of Advanced Melanoma Patients Treated with Ipilimumab. Clin Cancer Res. 2016; 22(12):2908-18.
18. Gonzalez S, Lopez-Soto A, Suarez-Alvarez B, Lopez-Vazquez A, and Lopez-Larrea C. NKG2D ligands: key targets of the immune response. Trends Immunol. 2008; 29(8):397-403.
19. Verhoeven D H, de Hooge A S, Mooiman E C, Santos S J, ten Dam M M, Gelderblom H, Melief C J, Hogendoorn P C, Egeler R M, van Tol M J, et al. NK cells recognize and lyse Ewing sarcoma cells through NKG2D and DNAM-1 receptor dependent pathways. Mol Immunol. 2008; 45(15):3917-25.
20. Boerman G H, van Ostaijen-ten Dam M M, Kraal K C, Santos S J, Ball L M, Lankester A C, Schilham M W, Egeler R M, and van Tol M J. Role of NKG2D, DNAM-1 and natural cytotoxicity receptors in cytotoxicity toward rhabdomyosarcoma cell lines mediated by resting and IL-15-activated human natural killer cells. Cancer immunology, immunotherapy: CII. 2015; 64(5):573-83.
21. McGary E C, Heimberger A, Mills L, Weber K, Thomas G W, Shtivelband M, Lev D C, and Bar-Eli M. A fully human antimelanoma cellular adhesion molecule/MUC18 antibody inhibits spontaneous pulmonary metastasis of osteosarcoma cells in vivo. Clin Cancer Res. 2003; 9(17):6560-6.
22. Charo J, Perez C, Buschow C, Jukica A, Czeh M, and Blankenstein T. Visualizing the dynamic of adoptively transferred T cells during the rejection of large established tumors. Eur J Immunol. 2011; 41(11):3187-97.
23. Quintarelli C, Vera J F, Savoldo B, Giordano Attianese G M, Pule M, Foster A E, Heslop H E, Rooney C M, Brenner M K, and Dotti G. Co-expression of cytokine and suicide genes to enhance the activity and safety of tumor-specific cytotoxic T lymphocytes. Blood. 2007; 110(8):2793-802.
24. Shum T, Omer B, Tashiro H, Kruse R L, Wagner D L, Parikh K, Yi Z, Sauer T, Liu D, Parihar R, et al. Constitutive Signaling from an Engineered IL7 Receptor Promotes Durable Tumor Elimination by Tumor-Redirected T Cells. Cancer Discov. 2017; 7(11):1238-47.
25. Kelly P F, Carrington J, Nathwani A, and Vanin E F. RD114-pseudotyped oncoretroviral vectors. Biological and physical properties. Ann N Y Acad Sci. 2001; 938(262-76; discussion 76-7.
26. Lapteva N, Parihar R, Rollins L A, Gee A P, and Rooney C M. Large-Scale Culture and Genetic Modification of Human Natural Killer Cells for Cellular Therapy. Methods Mol Biol. 2016; 1441(195-202.

What is claimed is:

1. A composition comprising two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof.

2. The composition of claim 1, wherein the first CAR polypeptide comprises:
the one or more antibodies or fragments thereof;
a transmembrane domain;
at least one costimulatory signaling region; and
optionally, a detection molecule.

3. The composition of claim 1 or 2, wherein the one or more cancer-associated antigens or binding regions thereof bound by the one or more antibodies or fragments thereof comprise CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, MUC18, NKG2D, NY-ESO-1, PSCA, WT-1, or any combination thereof.

4. The composition of any one of claims 1-3, wherein the one or more cancer-associated antigens or binding regions thereof bound by the one or more antibodies or fragments thereof is MUC18.

5. The composition of any one of claims 1-4, wherein the one or more antibodies are scFv monoclonal antibodies.

6. The composition of any one of claims 1-5, wherein the one or more antibodies are anti-MUC18 scFv monoclonal antibodies.

7. The composition of claim 6, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

8. The composition according to claim 6 or 7, wherein the anti-MUC18 antibody binds to MUC18 or a binding region thereof expressed by sarcoma cells, carcinoma cells, or a combination thereof.

9. The composition according to any one of claims 6-8, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

10. The composition according to any one of claims 6-8, wherein said anti-MUC18 antibody binds to MUC18 or a binding region thereof expressed by carcinoma cells comprising melanoma cells or prostatic adenocarcinoma cells.

11. The composition according to any one of claims 2-10, wherein the first CAR polypeptide does not comprise a CD3 zeta signaling domain.

12. The composition of any one of claims 2-11, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

13. The composition of any one of claims 2-12, wherein the at least one costimulatory signaling region comprises SEQ ID NO:8.

14. The composition of any one of claims 2-12, wherein the at least one costimulatory signaling region comprises SEQ ID NO:9.

15. The composition of any one of claims 2-12, wherein the at least one costimulatory signaling region comprises SEQ ID NO:10.

16. The composition of any one of claims 2-12, wherein the at least one costimulatory signaling region comprises SEQ ID NO:11.

17. The composition of any one of claims 2-16, wherein the one or more extracellular spacers comprise an IgG1 sequence, an IgG4, sequence, or a combination thereof.

18. The composition of any one of claims 2-17, wherein the transmembrane domain comprises SEQ ID NO:7.

19. The composition of any one of claims 1-18, wherein the first CAR polypeptide comprises an amino acid sequence having at least about 80% sequence identity to SEQ ID NO:12 or a fragment thereof.

20. The composition of any one of claims 1-18, wherein the first CAR polypeptide comprises an amino acid sequence having at least about 80% sequence identity to SEQ ID NO:13 or a fragment thereof.

21. The composition of any one of claims 1-18, wherein the first CAR polypeptide comprises an amino acid sequence having at least about 80% sequence identity to SEQ ID NO:14 or a fragment thereof.

22. The composition of any one of claims 1-18, wherein the first CAR polypeptide comprises an amino acid sequence having at least about 80% sequence identity to SEQ ID NO:15 or a fragment thereof.

23. The composition of any one of claims 1-22, wherein the second CAR polypeptide comprises:
the NKG2D receptor;
a CD3 zeta signaling domain; and
optionally, a detection molecule.

24. The composition of claim 23, wherein the extracellular domain of the NKG2D receptor binds to one or more NKG2D ligands comprising MICA, MICB, ULBP1, ULBP2, ULBP3, ULBP4, ULBP5, ULBP6, or a combination thereof.

25. The composition of claim 23 or claim 24, wherein the NKG2D receptor comprises an amino acid sequence having at least about 80% sequence identity to SEQ ID NO:17 or a fragment thereof.

26. The composition of any one of claims 23-25, wherein the CD3 zeta signaling domain comprises SEQ ID NO:16.

27. The composition of any one of claims 23-26, wherein the detection molecule comprises SEQ ID NO:18 and SEQ ID NO:19.

28. The composition of any one of claims 1-27, wherein the second CAR polypeptide comprises an amino acid sequence having at least about 80% sequence identity to SEQ ID NO:20 or a fragment thereof.

29. The composition as in any one of claims 1-28, wherein the first and/or second CAR polypeptides are encoded by one or more isolated nucleic acid sequences.

30. The composition of claim 29, wherein the one or more isolated nucleic acid sequences are comprised in one or more expression vectors.

31. The composition of claim 30, wherein the one or more expression vectors are lentiviral vectors, gamma-retroviral vectors, adenoviral vectors, adeno-associated viral vectors, or a combination thereof.

32. The composition of claim 30 or 31, wherein the expression vector is comprised in a cell and wherein the cell expresses a polypeptide encoded by the one or more isolated nucleic acid sequences comprised in the one or more expression vectors.

33. The composition of claim 32, wherein said cell is an immune effector cell.

34. The composition of claim 33, wherein the immune effector cell is selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof.

35. The composition of claim 34, wherein the immune effector cell is a Natural Killer (NK) cell.

36. The composition of any one of claims 32-35, wherein expression of the first and second CAR polypeptides by an immune effector cell increases the likelihood of NK activation at tumor sites.

37. The composition according to any one of claims 32-36, wherein the immune effector cell promotes an immune response against a cancer when the antibody of the first CAR polypeptide binds MUC18 or a binding region thereof on a target cell and/or the extracellular domain of the NKG2D receptor of the second CAR polypeptide binds one or more NKG2D ligand binding targets on the target cell.

38. The composition of claim 37, wherein the first and second CAR are encoded by the same isolated nucleic acid molecule.

39. The composition of any one of claims 1-38, wherein the cancer is a solid tumor cancer.

40. The composition of claim 39, wherein the solid tumor cancer is sarcoma, and wherein the sarcoma comprises rhabdomyosarcoma, Ewing sarcoma, clear cell sarcoma, or leiomyosarcoma.

41. The composition of claim 40, wherein the sarcoma is rhabdomyosarcoma.

42. The composition of claim 40, wherein the sarcoma is Ewing sarcoma.

43. The composition of claim 39, wherein the solid tumor cancer is carcinoma, and wherein the carcinoma comprises melanoma or prostatic adenocarcinoma.

44. A composition comprising an immune effector cell expressing two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof.

45. The composition of claim 44, wherein the first CAR polypeptide comprises:
the one or more antibodies or fragments thereof;
a transmembrane domain;
at least one costimulatory signaling region; and
optionally, a detection molecule.

46. The composition of claim 44 or 45, wherein the one or more cancer-associated antigens or binding regions thereof bound by the one or more antibodies or fragments thereof comprise CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, MUC18, NKG2D, NY-ESO-1, PSCA, WT-1, or any combination thereof.

47. The composition of any one of claims 44-46, wherein the one or more cancer-associated antigens or binding regions thereof bound by the one or more antibodies or fragments thereof is MUC18.

48. The composition of any one of claims 44-47, wherein the one or more antibodies are scFv monoclonal antibodies.

49. The composition of any one of claims 44-48, wherein the one or more antibodies are anti-MUC18 scFv monoclonal antibodies.

50. The composition of claim 49, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

51. The composition according to claim 49 or 50, wherein the anti-MUC18 antibody binds to MUC18 or a binding region thereof expressed by sarcoma cells, carcinoma cells, or a combination thereof.

52. The composition according to any one of claims 49-51, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

53. The composition according to any one of claims 49-51, wherein said anti-MUC18 antibody binds to MUC18 or a binding region thereof expressed by carcinoma cells comprising melanoma cells or prostatic adenocarcinoma cells.

54. The composition according to any one of claims 45-53, wherein the first CAR polypeptide does not comprise a CD3 zeta signaling domain.

55. The composition of any one of claims 45-54, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

56. The composition of any one of claims 45-55, wherein the at least one costimulatory signaling region comprises SEQ ID NO:8.

57. The composition of any one of claims 45-55, wherein the at least one costimulatory signaling region comprises SEQ ID NO:9.

58. The composition of any one of claims 45-55, wherein the at least one costimulatory signaling region SEQ ID NO:10.

59. The composition of any one of claims 45-55, wherein the at least one costimulatory signaling region comprises SEQ ID NO:11.

60. The composition of any one of claims 45-59, wherein the one or more extracellular spacers comprise an IgG1 sequence, an IgG4, sequence, or a combination thereof.

61. The composition of any one of claims 45-60, wherein the transmembrane domain comprises SEQ ID NO:7.

62. The composition of any one of claims 44-61, wherein the first CAR polypeptide comprises an amino acid sequence having at least about 80% sequence identity to SEQ ID NO:12 or a fragment thereof.

63. The composition of any one of claims 44-61, wherein the first CAR polypeptide comprises an amino acid sequence having at least about 80% sequence identity to SEQ ID NO:13 or a fragment thereof.

64. The composition of any one of claims 44-61, wherein the first CAR polypeptide comprises an amino acid sequence having at least about 80% sequence identity to SEQ ID NO:14 or a fragment thereof.

65. The composition of any one of claims 44-61, wherein the first CAR polypeptide comprises an amino acid sequence having at least about 80% sequence identity to SEQ ID NO:15 or a fragment thereof.

66. The composition of any one of claims 44-65, wherein the second CAR polypeptide comprises:
the NKG2D receptor;
a CD3 zeta signaling domain; and
optionally, a detection molecule.

67. The composition of claim 66, wherein the extracellular domain of the NKG2D receptor binds to one or more NKG2D ligands comprising MICA, MICB, ULBP1, ULBP2, ULBP3, ULBP4, ULBP5, ULBP6, or a combination thereof.

68. The composition of claim 66 or claim 67, wherein the NKG2D receptor comprises an amino acid sequence having at least about 80% sequence identity to SEQ ID NO:17 or a fragment thereof.

69. The composition of any one of claims 66-68, wherein the CD3 zeta signaling domain comprises SEQ ID NO:16.

70. The composition of any one of claims 66-69, wherein the detection molecule comprises SEQ ID NO:18 and SEQ ID NO:19.

71. The composition of any one of claims 44-70, wherein the second CAR polypeptide comprises an amino acid sequence having at least about 80% sequence identity to SEQ ID NO:20 or a fragment thereof.

72. The composition of any one of claims 44-71, wherein the immune effector cell is transfected with one or more expression vectors comprising one or more isolated nucleic acid sequences encoding the first and/or second CAR polypeptides.

73. The composition of claim 72, wherein the vector is a lentiviral vector, a gamma-retroviral vector, adenoviral vector, adeno-associated viral vector, or a combination thereof.

74. The composition of any one of claims 44-73, wherein the immune effector cell is selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof.

75. The composition of any one of claims 44-74, wherein the immune effector cell is a Natural Killer (NK) cell.

76. The composition of any one of claims 72-75, wherein expression of the first and second CAR polypeptides by an immune effector cell increases the likelihood of NK activation at tumor sites.

77. The composition of any one of claims 44-76, wherein the composition comprises a pharmaceutically acceptable carrier.

78. The composition of claims 44-77, wherein the amount of cells ranges from about 10⁴ up to about 10⁸ cells per kg body weight of the mammal.

79. The composition of any one of claims 44-78, wherein the immune effector cell promotes an immune response against a cancer when the antibody of the first CAR polypeptide binds MUC18 or a binding region thereof on a target cell and/or the extracellular domain of the NKG2D receptor of the second CAR polypeptide binds one or more NKG2D ligand binding targets on the target cell.

80. The composition of claim 79, wherein the first and second CAR are encoded by the same isolated nucleic acid molecule.

81. The composition of any one of claims 44-80, wherein the cancer is a solid tumor cancer.

82. The composition of claim 81, wherein the solid tumor cancer is sarcoma, and wherein the sarcoma comprises rhabdomyosarcoma, Ewing sarcoma, clear cell sarcoma, or leiomyosarcoma.

83. The composition of claim 82, wherein the sarcoma is rhabdomyosarcoma.

84. The composition of claim 82, wherein the sarcoma is Ewing sarcoma.

85. The composition of claim 81, wherein the solid tumor cancer is carcinoma, and wherein the carcinoma comprises melanoma or prostatic adenocarcinoma.

86. A method for stimulating an immune cell-mediated immune response to a target cell population and/or tissue in a mammal, wherein the target cell population and/or tissue express MUC18 and/or one or more NKG2D ligands, the method comprising administering to a mammal in need thereof a therapeutically effective amount of a composition comprising immune effector cells expressing two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof.

87. The method of claim 86, wherein the first CAR polypeptide comprises:
the one or more antibodies or fragments thereof;
a transmembrane domain;
at least one costimulatory signaling region; and
optionally, a detection molecule.

88. The method of claim 86 or 87, wherein the one or more cancer-associated antigens or binding regions thereof bound by the one or more antibodies or fragments thereof comprise CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, MUC18, NKG2D, NY-ESO-1, PSCA, WT-1, or any combination thereof.

89. The method of any one of claims 86-88, wherein the one or more cancer-associated antigens or binding regions thereof bound by the one or more antibodies or fragments thereof is MUC18.

90. The method of any one of claims 86-89, wherein the one or more antibodies are scFv monoclonal antibodies.

91. The method of claim 86-90, wherein the one or more antibodies are anti-MUC18 scFv monoclonal antibodies.

92. The method of claim 91, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

93. The method according to claim 91 or 92, wherein the anti-MUC18 antibody binds to MUC18 or a binding region thereof expressed by sarcoma cells, carcinoma cells, or a combination thereof.

94. The method according to any one of claims 91-93, wherein the anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

95. The method according to any one of claims 91-93, wherein said anti-MUC18 antibody binds to MUC18 or a binding region thereof expressed by carcinoma cells comprising melanoma cells or prostatic adenocarcinoma cells.

96. The method according to any one of claims 87-95, wherein the first CAR polypeptide does not comprise a CD3 zeta signaling domain.

97. The method of any one of claims 87-96, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

98. The method of any one of claims 87-97, wherein the at least one costimulatory signaling region comprises SEQ ID NO:8.

99. The method of any one of claims 87-97, wherein the at least one costimulatory signaling region comprises SEQ ID NO:9.

100. The method of any one of claims 87-97, wherein the at least one costimulatory signaling region comprises SEQ ID NO:10.

101. The method of any one of claims 87-97, wherein the at least one costimulatory signaling region comprises SEQ ID NO:11.

102. The method of any one of claims 87-101, wherein the one or more extracellular spacers comprise an IgG1 sequence, an IgG4, sequence, or a combination thereof.

103. The method of any one of claims 87-102, wherein the transmembrane domain comprises SEQ ID NO:7.

104. The method of any one of claims 87-103, wherein the first CAR polypeptide comprises an amino acid sequence having at least about 80% sequence identity to SEQ ID NO:12 or a fragment thereof.

105. The method of any one of claims 86-103, wherein the first CAR polypeptide comprises an amino acid sequence having at least about 80% sequence identity to SEQ ID NO:13 or a fragment thereof.

106. The method of any one of claims 86-103, wherein the first CAR polypeptide comprises an amino acid sequence having at least about 80% sequence identity to SEQ ID NO:14 or a fragment thereof.

107. The method of any one of claims 86-103, wherein the first CAR polypeptide comprises an amino acid sequence having at least about 80% sequence identity to SEQ ID NO:15 or a fragment thereof.

108. The method of any one of claims 86-107, wherein the second CAR polypeptide comprises:
the NKG2D receptor;
a CD3 zeta signaling domain; and
optionally, a detection molecule.

109. The method of claim 108, wherein the extracellular domain of the NKG2D receptor binds to one or more NKG2D ligands comprising MICA, MICB, ULBP1, ULBP2, ULBP3, ULBP4, ULBP5, ULBP6, or a combination thereof.

110. The method of claim 108 or claim 109, wherein the extracellular domain of the NKG2D receptor comprises an amino acid sequence having at least about 80% sequence identity to SEQ ID NO:17 or a fragment thereof.

111. The method of any one of claims 108-110, wherein the CD3 zeta signaling domain comprises SEQ ID NO:16.

112. The method of any one of claims 108-111, wherein the detection molecule comprises SEQ ID NO:18 and SEQ ID NO:19.

113. The method of any of one of claims 86-111, wherein the second CAR polypeptide comprises an amino acid sequence having at least about 80% sequence identity to SEQ ID NO:20 or a fragment thereof.

114. The method of any one of claims 86-113, wherein the immune effector cells are transfected with one or more expression vectors comprising one or more isolated nucleic acid sequences encoding the first and/or second CAR polypeptides.

115. The method of claim 114, wherein the vector is a lentiviral vector, a gamma-retroviral vector, adenoviral vector, adeno-associated viral vector, or a combination thereof.

116. The method of any one of claims 86-115, wherein the immune effector cells are selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof.

117. The method of any one of claims 86-116, wherein the immune effector cells are Natural Killer (NK) cells.

118. The method of any one of claims 114-117, wherein expression of the first and second CAR polypeptides by immune effector cells increases the likelihood of NK activation at tumor sites.

119. The method of any one of claims 86-118, wherein the composition comprises a pharmaceutically acceptable carrier.

120. The method of claim 86-119, wherein the amount of cells ranges from about 10⁴ up to about 10⁸ cells per kg body weight of the mammal.

121. The method of any one of claims 86-120, wherein the immune effector cells promote an immune response against a cancer when the antibody of the first CAR polypeptide binds MUC18 or a binding region thereof on a target cell and/or the extracellular domain of the NKG2D receptor of the second CAR polypeptide binds one or more NKG2D ligand binding targets on the target cell.

122. The method of claim 121, wherein the first and second CAR are encoded by the same isolated nucleic acid molecule.

123. The method of any one of claim 86-122, wherein the target cell population and/or tissue comprises solid tumor cancer cells and/or tissue.

124. The method of claim 123, wherein the solid tumor cancer cells and/or tissue comprises sarcoma cells and/or tissue, carcinoma cells and/or tissue, or a combination thereof.

125. The method of claim 124, wherein the sarcoma cells and/or tissue comprises rhabdomyosarcoma, Ewing sarcoma, clear cell sarcoma, or leiomyosarcoma cells and/or tissue.

126. The method of claim 124, wherein the carcinoma cells and/or tissue comprises melanoma or prostatic adenocarcinoma cells and/or tissue.

127. A method of generating a persisting population of genetically engineered immune cells in a mammal, said method comprising administering to the mammal a therapeutically effective amount of a composition comprising immune effector cells genetically modified to express two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof, wherein the persisting population of genetically engineered immune cells persists in the mammal for at least about one month up to at least about one year after administration.

128. The method of claim 127, wherein the first CAR polypeptide comprises:
the one or more antibodies or fragments thereof;
a transmembrane domain;
at least one costimulatory signaling region; and
optionally, a detection molecule.

129. The method of claim 127 or 128, wherein the one or more cancer-associated antigens or binding regions thereof bound by the one or more antibodies or fragments thereof comprise CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, MUC18, NKG2D, NY-ESO-1, PSCA, WT-1, or any combination thereof.

130. The method of any one of claims 127-129, wherein the one or more cancer-associated antigens or binding regions thereof bound by the one or more antibodies or fragments thereof is MUC18.

131. The method of any one of claims 127-130, wherein the one or more antibodies are scFv monoclonal antibodies.

132. The method of any one of claims 127-131, wherein the one or more antibodies are anti-MUC18 scFv monoclonal antibodies.

133. The method of claim 132, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

134. The method according to claim 132 or 133, wherein the anti-MUC18 antibody binds to MUC18 or a binding region thereof expressed by sarcoma cells, carcinoma cells, or a combination thereof.

135. The method according to any one of claims 132-134, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

136. The method according to any one of claims 132-134, wherein said anti-MUC18 antibody binds to MUC18 or a binding region thereof expressed carcinoma cells comprising melanoma cells or prostatic adenocarcinoma cells.

137. The method according to any one of claims 128-136, wherein the first CAR polypeptide does not comprise a CD3 zeta signaling domain.

138. The method of any one of claims 128-137, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

139. The method of any one of claims 128-138, wherein the at least one costimulatory signaling region comprises SEQ ID NO:8.

140. The method of any one of claims 128-138, wherein the at least one costimulatory signaling region comprises SEQ ID NO:9.

141. The method of any one of claims 128-138, wherein the at least one costimulatory signaling region comprises SEQ ID NO:10.

142. The method of any one of claims 128-138, wherein the at least one costimulatory signaling region comprises SEQ ID NO:11.

143. The method of any one of claims 128-142, wherein the one or more extracellular spacers comprise an IgG1 sequence, an IgG4, sequence, or a combination thereof.

144. The method of any one of claims 128-143, wherein the transmembrane domain is a CD28-derived sequence.

145. The method of any one of claims 128-144, wherein the first CAR polypeptide comprises an amino acid sequence having at least about 80% sequence identity to SEQ ID NO:12 or a fragment thereof.

146. The method of any one of claims 127-144, wherein the first CAR polypeptide comprises an amino acid sequence having at least about 80% sequence identity to SEQ ID NO:13 or a fragment thereof.

147. The method of any one of claims 127-144, wherein the first CAR polypeptide comprises an amino acid sequence having at least about 80% sequence identity to SEQ ID NO:14 or a fragment thereof.

148. The method of any one of claims 127-144, wherein the first CAR polypeptide comprises an amino acid sequence having at least about 80% sequence identity to SEQ ID NO:15 or a fragment thereof.

149. The method of any one of claims 127-148, wherein the second CAR polypeptide comprises:
the NKG2D receptor;
a CD3 zeta signaling domain; and
optionally, a detection molecule.

150. The method of claim 149, wherein the extracellular domain of the NKG2D receptor binds to one or more NKG2D ligands comprising MICA, MICB, ULBP1, ULBP2, ULBP3, ULBP4, ULBP5, ULBP6, or a combination thereof.

151. The method of claim 149 or claim 150, wherein the extracellular domain of the NKG2D receptor comprises an amino acid sequence having at least about 80% sequence identity to SEQ ID NO:17 or a fragment thereof.

152. The method of any one of claims 149-151, wherein the CD3 zeta signaling domain comprises SEQ ID NO:16.

153. The method of any one of claims 149-152, wherein the detection molecule comprises SEQ ID NO:18 and SEQ ID NO:19.

154. The method of any of one of claims 127-153, wherein the second CAR polypeptide comprises an amino acid sequence having at least about 80% sequence identity to SEQ ID NO:20 or a fragment thereof.

155. The method of any one of claims 127-154, wherein the immune effector cells are transfected with one or more expression vectors comprising one or more isolated nucleic acid sequences encoding the first and/or second CAR polypeptides.

156. The method of claim 155, wherein the vector is a lentiviral vector, a gamma-retroviral vector, adenoviral vector, adeno-associated viral vector, or a combination thereof.

157. The method of any one of claims 127-156, wherein the immune effector cells are selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof.

158. The method of any one of claims 127-157, wherein the immune effector cells are Natural Killer (NK) cells.

159. The method of any one of claims 155-158, wherein expression of the first and second CAR polypeptides by immune effector cells increases the likelihood of NK activation at tumor sites.

160. The method of any one of claims 127-159, wherein the composition comprises a pharmaceutically acceptable carrier.

161. The method of claim 127-160, wherein the amount of cells ranges from about 10⁴ up to about 10⁸ cells per kg body weight of the mammal.

162. The method of any one of claims 127-161, wherein the immune effector cells promote an immune response against a cancer when the antibody of the first CAR polypeptide binds MUC18 or a binding region thereof on a target cell and/or the extracellular domain of the NKG2D receptor of the second CAR polypeptide binds one or more NKG2D ligand binding targets on the target cell.

163. The method of claim 162, wherein the first and second CAR are encoded by the same isolated nucleic acid molecule.

164. The method of any one of claims 127-163, wherein the cancer is a solid tumor cancer.

165. The method of claim 164, wherein the solid tumor cancer is sarcoma, and wherein the sarcoma comprises rhabdomyosarcoma, Ewing sarcoma, clear cell sarcoma, or leiomyosarcoma.

166. The method of claim 165, wherein the sarcoma is rhabdomyosarcoma.

167. The method of claim 165, wherein the sarcoma is Ewing sarcoma.

168. The method of claim 164, wherein the solid tumor cancer is carcinoma, and wherein the carcinoma comprises melanoma or prostatic adenocarcinoma.

169. A method of expanding a population of genetically engineered immune cells in a mammal, the method comprising administering to the mammal a therapeutically effective amount of a composition comprising immune effector cells genetically modified to express two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof, wherein the administered genetically engineered immune cell produces a population of progeny immune cells in the mammal.

170. The method of claim 169, wherein the first CAR polypeptide comprises:
the one or more antibodies or fragments thereof;
a transmembrane domain;
at least one costimulatory signaling region; and
optionally, a detection molecule.

171. The method of claim 169 or 170, wherein the one or more cancer-associated antigens or binding regions thereof bound by the one or more antibodies or fragments thereof comprise CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, MUC18, NKG2D, NY-ESO-1, PSCA, WT-1, or any combination thereof.

172. The method of any one of claims 169-171, wherein the one or more cancer-associated antigens or binding regions thereof bound by the one or more antibodies or fragments thereof is MUC18.

173. The method of any one of claims 169-172, wherein the one or more antibodies are scFv monoclonal antibodies.

174. The method of claim 173, wherein the one or more antibodies are anti-MUC18 scFv monoclonal antibodies.

175. The method of claim 174, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

176. The method according to claim 174 or 175, wherein the anti-MUC18 antibody binds to MUC18 or a binding region thereof expressed by sarcoma cells, carcinoma cells, or a combination thereof.

177. The method according to any one of claims 174-176, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

178. The method according to any one of claims 174-176, wherein said anti-MUC18 antibody binds to MUC18 or a binding region thereof expressed by carcinoma cells comprising melanoma cells or prostatic adenocarcinoma cells.

179. The method according to any one of claims 170-178, wherein the first CAR polypeptide does not comprise a CD3 zeta signaling domain.

180. The method of any one of claims 170-179, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

181. The method of any one of claims 170-180, wherein the at least one costimulatory signaling region comprises SEQ ID NO:8.

182. The method of any one of claims 170-180, wherein the at least one costimulatory signaling region comprises SEQ ID NO:9.

183. The method of any one of claims 170-180, wherein the at least one costimulatory signaling region comprises SEQ ID NO:10.

184. The method of any one of claims 170-180, wherein the at least one costimulatory signaling region comprises SEQ ID NO:11.

185. The method of any one of claims 170-184, wherein the one or more extracellular spacers comprise an IgG1 sequence, an IgG4, sequence, or a combination thereof.

186. The method of any one of claims 170-185, wherein the transmembrane domain comprises SEQ ID NO:7.

187. The method of any one of claims 169-186, wherein the first CAR polypeptide comprises an amino acid sequence having at least about 80% sequence identity to SEQ ID NO:12 or a fragment thereof.

188. The method of any one of claims 169-186, wherein the first CAR polypeptide comprises an amino acid sequence having at least about 80% sequence identity to SEQ ID NO:13 or a fragment thereof.

189. The method of any one of claims 169-186, wherein the first CAR polypeptide comprises an amino acid sequence having at least about 80% sequence identity to SEQ ID NO:14 or a fragment thereof.

190. The method of any one of claims 169-186, wherein the first CAR polypeptide comprises an amino acid sequence having at least about 80% sequence identity to SEQ ID NO:15 or a fragment thereof.

191. The method of any one of claims 169-190, wherein the second CAR polypeptide comprises:
the NKG2D receptor;
a CD3 zeta signaling domain; and
optionally, a detection molecule.

192. The method of claim 191, wherein the extracellular domain of the NKG2D receptor binds to one or more NKG2D ligands comprising MICA, MICB, ULBP1, ULBP2, ULBP3, ULBP4, ULBP5, ULBP6, or a combination thereof.

193. The method of claim 191 or claim 192, wherein the extracellular domain of the NKG2D receptor comprises an amino acid sequence having at least about 80% sequence identity to SEQ ID NO:17 or a fragment thereof.

194. The method of any one of claims 191-193, wherein the CD3 zeta signaling domain comprises SEQ ID NO:16.

195. The method of any one of claims 191-194, wherein the detection molecule comprises SEQ ID NO:18 and SEQ ID NO:19.

196. The method of any of one of claims 169-195, wherein the second CAR polypeptide comprises an amino acid sequence having at least about 80% sequence identity to SEQ ID NO:20 or a fragment thereof.

197. The method of any one of claims 169-196, wherein the immune effector cells are transfected with one or more expression vectors comprising one or more isolated nucleic acid sequences encoding the first and/or second CAR polypeptides.

198. The method of claim 197, wherein the vector is a lentiviral vector, a gamma-retroviral vector, adenoviral vector, adeno-associated viral vector, or a combination thereof.

199. The method of any one of claims 169-198, wherein the immune effector cells are selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

200. The method of any one of claims 169-199, wherein the immune effector cell are Natural Killer (NK) cells.

201. The method of any one of claims 197-200, wherein expression of the first and second CAR polypeptides by immune effector cells increases the likelihood of NK activation at tumor sites.

202. The method of any one of claims 169-201, wherein the composition comprises a pharmaceutically acceptable carrier.

203. The method of claim 169-202, wherein the amount of cells ranges from about 10⁴ up to about 10⁸ cells per kg body weight of the mammal.

204. The method of any one of claims 169-203, wherein the immune effector cells promote an immune response against a cancer when the antibody of the first CAR polypeptide binds MUC18 or a binding region thereof on a target cell and/or the extracellular domain of the NKG2D receptor of the second CAR polypeptide binds one or more NKG2D ligand binding targets on the target cell.

205. The composition of claim 204, wherein the first and second CAR are encoded by the same isolated nucleic acid molecule.

206. The method of any one of claims 169-205, wherein the cancer is a solid tumor cancer.

207. The method of claim 206, wherein the solid tumor cancer is sarcoma, and wherein the sarcoma comprises rhabdomyosarcoma, Ewing sarcoma, clear cell sarcoma, or leiomyosarcoma.

208. The method of claim 207, wherein the sarcoma is rhabdomyosarcoma.

209. The method of claim 207, wherein the sarcoma is Ewing sarcoma.

210. The method of claim 206, wherein the solid tumor cancer is carcinoma, and wherein the carcinoma comprises melanoma or prostatic adenocarcinoma.

211. A method of treating a mammal with a cancer comprising cells that express MUC18 and/or one or more NKG2D ligands, the method comprising administering to a mammal in need thereof a therapeutically effective amount of a composition comprising immune effector cells genetically modified to express two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof.

212. The method of claim 211, wherein the first CAR polypeptide comprises:
the one or more antibodies or fragments thereof;
a transmembrane domain;
at least one costimulatory signaling region; and
optionally, a detection molecule.

213. The method of claim 211 or 212, wherein the one or more cancer-associated antigens or binding regions thereof bound by the one or more antibodies or fragments thereof comprise CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, MUC18, NKG2D, NY-ESO-1, PSCA, WT-1, or any combination thereof.

214. The method of any one of claims 211-213, wherein the one or more cancer-associated antigens or binding regions thereof bound by the one or more antibodies or fragments thereof is MUC18.

215. The method of any one of claims 211-214, wherein the one or more antibodies are scFv monoclonal antibodies.

216. The method of claim 215, wherein the one or more antibodies are anti-MUC18 scFv monoclonal antibodies.

217. The method of claim 216, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

218. The method according to claim 216 or 217, wherein the anti-MUC18 antibody binds to MUC18 or a binding region thereof expressed by sarcoma cells, carcinoma cells, or a combination thereof.

219. The method according to any one of claims 216-218, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

220. The method according to any one of claims 216-218, wherein said anti-MUC18 antibody binds to MUC18 or a binding region thereof expressed by carcinoma cells comprising melanoma cells or prostatic adenocarcinoma cells.

221. The method according to any one of claims 212-220, wherein the first CAR polypeptide does not comprise a CD3 zeta signaling domain.

222. The method of any one of claims 212-221, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

223. The method of any one of claims 212-222, wherein the at least one costimulatory signaling region comprises SEQ ID NO:8.

224. The method of any one of claims 212-222, wherein the at least one costimulatory signaling region comprises SEQ ID NO:9.

225. The method of any one of claims 212-222, wherein the at least one costimulatory signaling region comprises SEQ ID NO:10.

226. The method of any one of claims 212-222, wherein the at least one costimulatory signaling region comprises SEQ ID NO:11.

227. The method of any one of claims 212-226, wherein the one or more extracellular spacers comprise an IgG1 sequence, an IgG4, sequence, or a combination thereof.

228. The method of any one of claims 212-227, wherein the transmembrane domain comprises SEQ ID NO:7.

229. The method of any one of claims 211-228, wherein the first CAR polypeptide comprises an amino acid sequence having at least about 80% sequence identity to SEQ ID NO:12 or a fragment thereof.

230. The method of any one of claims 211-228, wherein the first CAR polypeptide comprises an amino acid sequence having at least about 80% sequence identity to SEQ ID NO:13 or a fragment thereof.

231. The method of any one of claims 211-228, wherein the first CAR polypeptide comprises an amino acid sequence having at least about 80% sequence identity to SEQ ID NO:14 or a fragment thereof.

232. The method of any one of claims 211-228, wherein the first CAR polypeptide comprises an amino acid sequence having at least about 80% sequence identity to SEQ ID NO:15 or a fragment thereof.

233. The method of any one of claims 211-232, wherein the second CAR polypeptide comprises:
the NKG2D receptor;
a CD3 zeta signaling domain; and
optionally, a detection molecule.

234. The method of claim 233, wherein the extracellular domain of the NKG2D receptor binds to one or more NKG2D ligands comprising MICA, MICB, ULBP1, ULBP2, ULBP3, ULBP4, ULBP5, ULBP6, or a combination thereof.

235. The method of claim 233 or claim 234, wherein the extracellular domain of the NKG2D receptor comprises an amino acid sequence having at least about 80% sequence identity to SEQ ID NO:17 or a fragment thereof.

236. The method of any one of claims 233-235, wherein the CD3 zeta signaling domain comprises SEQ ID NO:16.

237. The method of any one of claims 233-236, wherein the detection molecule comprises SEQ ID NO:18 and SEQ ID NO:19.

238. The method of any of one of claims 211-237, wherein the second CAR polypeptide comprises an amino acid sequence having at least about 80% sequence identity to SEQ ID NO:20 or a fragment thereof.

239. The method of any one of claims 211-238, wherein the immune effector cells are transfected with one or more expression vectors comprising one or more isolated nucleic acid sequences encoding the first and/or second CAR polypeptides.

240. The method of claim 239, wherein the vector is a lentiviral vector, a gamma-retroviral vector, adenoviral vector, adeno-associated viral vector, or a combination thereof.

241. The method of any one of claims 211-240, wherein the immune effector cells are selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof.

242. The method of any one of claims 211-241, wherein the immune effector cells are Natural Killer (NK) cells.

243. The method of any one of claims 239-242, wherein expression of the first and second CAR polypeptides by immune effector cells increases the likelihood of NK activation at tumor sites.

244. The method of any one of claims 211-243, wherein the composition comprises a pharmaceutically acceptable carrier.

245. The method of claim 211-244, wherein the amount of cells ranges from about 10⁴ up to about 10⁸ cells per kg body weight of the mammal.

246. The method of any one of claims 211-245, wherein the immune effector cells promote an immune response against a cancer when the antibody of the first CAR polypeptide binds MUC18 or a binding region thereof on a target cell and/or the extracellular domain of the NKG2D receptor of the second CAR polypeptide binds one or more NKG2D ligand binding targets on the target cell.

247. The composition of claim 246, wherein the first and second CAR are encoded by the same isolated nucleic acid molecule.

248. The method of any one of claims 211-247, wherein the cancer is a solid tumor cancer.

249. The method of claim 248, wherein the solid tumor cancer is sarcoma, and wherein the sarcoma comprises rhabdomyosarcoma, Ewing sarcoma, clear cell sarcoma, or leiomyosarcoma.

250. The method of claim 249, wherein the sarcoma is rhabdomyosarcoma.

251. The method of claim 249, wherein the sarcoma is Ewing sarcoma.

252. The method of claim 248, wherein the solid tumor cancer is carcinoma, and wherein the carcinoma comprises melanoma or prostatic adenocarcinoma.

253. A method for treatment of cancer comprising the steps of contacting an immune effector cell with a cancer cell or MDSC, M2-TAM, or Treg within the cancer microenvironment of a mammal and inducing apoptosis of the cancer cell, wherein the immune effector cell is genetically modified to express two chimeric antigen receptor (CAR) polypeptides, the first CAR polypeptide comprising one or more antibodies or fragments thereof that binds to one or more cancer-associated antigens or a binding region thereof, and the second CAR polypeptide comprising an NKG2D receptor or fragment thereof.

254. The method of claim 253, wherein the first CAR polypeptide comprises:
the one or more antibodies or fragments thereof;
a transmembrane domain;
at least one costimulatory signaling region; and
optionally, a detection molecule.

255. The method of claim 253 or 254, wherein the one or more cancer-associated antigens or binding regions thereof bound by the one or more antibodies or fragments thereof comprise CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, MUC18, NKG2D, NY-ESO-1, PSCA, WT-1, or any combination thereof.

256. The method of any one of claims 253-255, wherein the one or more cancer-associated antigens or binding regions thereof bound by the one or more antibodies or fragments thereof is MUC18.

257. The method of any one of claims 253-256, wherein the one or more antibodies are scFv monoclonal antibodies.

258. The method of any one of claims 253-256, wherein the one or more antibodies are anti-MUC18 scFv monoclonal antibodies.

259. The method of claim 258, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

260. The method according to claim 258 or 259, wherein the anti-MUC18 antibody binds to MUC18 or a binding region thereof expressed by sarcoma cells, carcinoma cells, or a combination thereof.

261. The method according to any one of claim 258 or 260, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

262. The method according to any one of claims 258-260, wherein said anti-MUC18 antibody binds to MUC18 or a binding region thereof expressed by carcinoma cells comprising melanoma cells or prostatic adenocarcinoma cells.

263. The method according to any one of claims 254-262, wherein the first CAR polypeptide does not comprise a CD3 zeta signaling domain.

264. The method of any one of claims 254-263, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

265. The method of any one of claims 254-264, wherein the at least one costimulatory signaling region comprises SEQ ID NO:8.

266. The method of any one of claims 254-264, wherein the at least one costimulatory signaling region comprises SEQ ID NO:9.

267. The method of any one of claims 254-264, wherein the at least one costimulatory signaling region comprises SEQ ID NO:10.

268. The method of any one of claims 254-264, wherein the at least one costimulatory signaling region comprises SEQ ID NO:11.

269. The method of any one of claims 254-268, wherein the one or more extracellular spacers comprise an IgG1 sequence, an IgG4, sequence, or a combination thereof.

270. The method of any one of claims 254-269, wherein the transmembrane domain comprises SEQ ID NO:7.

271. The method of any one of claims 253-270, wherein the first CAR polypeptide comprises an amino acid sequence having at least about 80% sequence identity to SEQ ID NO:12 or a fragment thereof.

272. The method of any one of claims 253-270, wherein the first CAR polypeptide comprises an amino acid sequence having at least about 80% sequence identity to SEQ ID NO:13 or a fragment thereof.

273. The method of any one of claims 253-270, wherein the first CAR polypeptide comprises an amino acid sequence having at least about 80% sequence identity to SEQ ID NO:14 or a fragment thereof.

274. The method of any one of claims 253-270, wherein the first CAR polypeptide comprises an amino acid sequence having at least about 80% sequence identity to SEQ ID NO:15 or a fragment thereof.

275. The method of any one of claims 253-274, wherein the second CAR polypeptide comprises:
the NKG2D receptor;
a CD3 zeta signaling domain; and
optionally, a detection molecule.

276. The method of claim 275, wherein the extracellular domain of the NKG2D receptor binds to one or more NKG2D ligands comprising MICA, MICB, ULBP1, ULBP2, ULBP3, ULBP4, ULBP5, ULBP6, or a combination thereof.

277. The method of claim 275 or claim 276, wherein the extracellular domain of the NKG2D receptor comprises an amino acid sequence having at least about 80% sequence identity to SEQ ID NO:17 or a fragment thereof.

278. The method of any one of claims 275-277, wherein the CD3 zeta signaling domain comprises SEQ ID NO:16.

279. The method of any one of claims 275-278, wherein the detection molecule comprises SEQ ID NO:18 and SEQ ID NO:19.

280. The method of any of one of claims 253-279, wherein the second CAR polypeptide comprises an amino acid sequence having at least about 80% sequence identity to SEQ ID NO:20 or a fragment thereof.

281. The method of any one of claims 253-280, wherein the immune effector cell is transfected with one or more expression vectors comprising one or more isolated nucleic acid sequences encoding the first and/or second CAR polypeptides.

282. The method of claim 281, wherein the vector is a lentiviral vector, a gamma-retroviral vector, adenoviral vector, adeno-associated viral vector, or a combination thereof.

283. The method of any one of claims 253-282, wherein the immune effector cell is selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

284. The method of any one of claims 253-283, wherein the immune effector cell is a Natural Killer (NK) cell.

285. The method of any one of claims 281-284, wherein expression of the first and second CAR polypeptides by an immune effector cell increases the likelihood of NK activation at tumor sites.

286. The method of any one of claims 253-285, wherein the composition comprises a pharmaceutically acceptable carrier.

287. The method of claim 286, wherein the amount of cells ranges from about 10⁴ up to about 10⁸ cells per kg body weight of the mammal.

288. The method of any one of claims 253-287, wherein the immune effector cell promotes an immune response against a cancer when the antibody of the first CAR polypeptide binds MUC18 or a binding region thereof on a target cell and/or the extracellular domain of the NKG2D receptor of the second CAR polypeptide binds one or more NKG2D ligand binding targets on the target cell.

289. The composition of claim 288, wherein the first and second CAR are encoded by the same isolated nucleic acid molecule.

290. The method of any one of claims 253-289, wherein the cancer is a solid tumor cancer.

291. The method of claim 290, wherein the solid tumor cancer is sarcoma, and wherein the sarcoma comprises rhabdomyosarcoma, Ewing sarcoma, clear cell sarcoma, or leiomyosarcoma.

292. The method of claim 291, wherein the sarcoma is rhabdomyosarcoma.

293. The method of claim 291, wherein the sarcoma is Ewing sarcoma.

294. The method of claim 290, wherein the solid tumor cancer is carcinoma, and wherein the carcinoma comprises melanoma or prostatic adenocarcinoma.

295. A composition comprising a chimeric antigen receptor (CAR) polypeptide comprising one or more antibodies or fragments thereof that binds to one or more cancer-associated antigens comprising MUC18, CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, NKG2D, NY-ESO-1, PSCA, WT-1, or binding regions thereof.

296. The composition of claim 295, wherein the CAR polypeptide comprises:
the one or more antibodies or fragments thereof;
one or more extracellular spacers;
a transmembrane domain;
at least one costimulatory signaling region; and
optionally, a detection molecule.

297. The composition of claim 295 or 296, wherein the one or more cancer-associated antigens or binding regions thereof bound by the one or more antibodies or fragments thereof is MUC18.

298. The composition of any one of claims 295-297, wherein the one or more antibodies are scFv monoclonal antibodies.

299. The composition of any one of claims 295-298, wherein the one or more antibodies are anti-MUC18 scFv monoclonal antibodies.

300. The composition of claim 299, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

301. The composition according to claim 299 or 300, wherein the anti-MUC18 antibody binds to MUC18 or a binding region thereof expressed by sarcoma cells, carcinoma cells, or a combination thereof.

302. The composition according to any one of claims 299-301, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

303. The composition according to any one of claims 299-301, wherein said anti-MUC18 antibody binds to MUC18 or a binding region thereof expressed by carcinoma cells comprising melanoma cells or prostatic adenocarcinoma cells.

304. The composition according to any one of claims 296-303, wherein the CAR polypeptide does not comprise a CD3 zeta signaling domain.

305. The composition of any one of claims 296-304, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

306. The composition of any one of claims 296-305, wherein the at least one costimulatory signaling region comprises SEQ ID NO:8.

307. The composition of any one of claims 296-305, wherein the at least one costimulatory signaling region comprises SEQ ID NO:9.

308. The composition of any one of claims 296-305, wherein the at least one costimulatory signaling region comprises SEQ ID NO:10.

309. The composition of any one of claims 296-305, wherein the at least one costimulatory signaling region comprises SEQ ID NO:11.

310. The composition of any one of claims 296-309, wherein the one or more extracellular spacers comprise an IgG1 sequence, an IgG4, sequence, or a combination thereof.

311. The composition of any one of claims 296-310, wherein the transmembrane domain comprises SEQ ID NO:7.

312. The composition of any one of claims 295-311, wherein the CAR polypeptide comprises an amino acid sequence having at least about 80% sequence identity to SEQ ID NO:12 or a fragment thereof.

313. The composition of any one of claims 295-311, wherein the CAR polypeptide comprises an amino acid sequence having at least about 80% sequence identity to SEQ ID NO:13 or a fragment thereof.

314. The composition of any one of claims 295-311, wherein the CAR polypeptide comprises an amino acid sequence having at least about 80% sequence identity to SEQ ID NO:14 or a fragment thereof.

315. The composition of any one of claims 295-311, wherein the CAR polypeptide comprises an amino acid sequence having at least about 80% sequence identity to SEQ ID NO:15 or a fragment thereof.

316. The composition as in any one of claims 295-315, wherein the CAR polypeptide is encoded by an isolated nucleic acid sequence.

317. The composition of claim 316, wherein the isolated nucleic acid sequence is comprised in an expression vector.

318. The composition of claim 317, wherein the expression vector is a lentiviral vector, a gamma-retroviral vector, an adenoviral vector, or an adeno-associated viral vector.

319. The composition of claim 317 or 318, wherein the expression vector is comprised in a cell and wherein the cell expresses a polypeptide encoded by the one or more isolated nucleic acid sequences comprised in the one or more expression vectors.

320. The composition of claim 319, wherein said cell is an immune effector cell.

321. The composition of claim 320, wherein the immune effector cell is selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof.

322. The composition of claim 321, wherein the immune effector cell is a Natural Killer (NK) cell.

323. The composition of any one of claims 295-322, wherein the cancer is a solid tumor cancer.

324. The composition of claim 323, wherein the solid tumor cancer is sarcoma, and wherein the sarcoma comprises rhabdomyosarcoma, Ewing sarcoma, clear cell sarcoma, or leiomyosarcoma.

325. The composition of claim 324, wherein the sarcoma is rhabdomyosarcoma.

326. The composition of claim 324, wherein the sarcoma is Ewing sarcoma.

327. The composition of claim 323, wherein the solid tumor cancer is carcinoma, and wherein the carcinoma comprises melanoma or prostatic adenocarcinoma.

328. The composition of any one of claims 295-327, further comprising a second CAR polypeptide comprising an NKG2D receptor or fragment thereof.

329. The composition of claim 328, wherein expression of the first and second CAR polypeptides by an immune effector cell increases the likelihood of NK activation at tumor sites.

330. The composition according to claim 329, wherein the immune effector cell promotes an immune response against a cancer when the antibody of the CAR polypeptide binds MUC18 or a binding region thereof on a target cell and/or the extracellular domain of the NKG2D receptor of the second CAR polypeptide binds one or more NKG2D ligand binding targets on the target cell.

331. A composition comprising an immune effector cell expressing a chimeric antigen receptor (CAR) polypeptide, the CAR polypeptide comprising one or more antibodies or fragments thereof that binds to one or more cancer-associated antigens comprising MUC18, CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, NKG2D, NY-ESO-1, PSCA, WT-1, or binding regions thereof.

332. The composition of claim 331, wherein the CAR polypeptide comprises:
the one or more antibodies or fragments thereof;
one or more extracellular spacers;
a transmembrane domain;
at least one costimulatory signaling region; and
optionally, a detection molecule.

333. The composition of claim 331 or 332, wherein the one or more cancer-associated antigens or binding regions thereof bound by the one or more antibodies or fragments thereof is MUC18.

334. The composition of any one of claims 331-333, wherein the one or more antibodies are scFv monoclonal antibodies.

335. The composition of any one of claims 331-334, wherein the one or more antibodies are anti-MUC18 scFv monoclonal antibodies.

336. The composition of claim 335, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

337. The composition according to claim 335 or 336, wherein the anti-MUC18 antibody binds to MUC18 or a binding region thereof expressed by sarcoma cells, carcinoma cells, or a combination thereof.

338. The composition according to any one of claims 335-337, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

339. The composition according to any one of claims 335-337, wherein said anti-MUC18 antibody binds to MUC18 or a binding region thereof expressed by carcinoma cells comprising melanoma cells or prostatic adenocarcinoma cells.

340. The composition according to any one of claims 332-339, wherein the CAR polypeptide does not comprise a CD3 zeta signaling domain.

341. The composition of any one of claims 332-340, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

342. The composition of any one of claims 332-341, wherein the at least one costimulatory signaling region comprises SEQ ID NO:8.

343. The composition of any one of claims 332-341, wherein the at least one costimulatory signaling region comprises SEQ ID NO:9.

344. The composition of any one of claims 332-341, wherein the at least one costimulatory signaling region comprises SEQ ID NO:10.

345. The composition of any one of claims 332-341, wherein the at least one costimulatory signaling region comprises SEQ ID NO:11.

346. The composition of any one of claims 332-345, wherein the one or more extracellular spacers comprise an IgG1 sequence, an IgG4, sequence, or a combination thereof.

347. The composition of any one of claims 332-346, wherein the transmembrane domain comprises SEQ ID NO:7

348. The composition of any one of claims 331-347, wherein the CAR polypeptide comprises an amino acid sequence having at least about 80% sequence identity to SEQ ID NO:12 or a fragment thereof.

349. The composition of any one of claims 331-347, wherein the CAR polypeptide comprises an amino acid sequence having at least about 80% sequence identity to SEQ ID NO:13 or a fragment thereof.

350. The composition of any one of claims 331-347, wherein the CAR polypeptide comprises an amino acid sequence having at least about 80% sequence identity to SEQ ID NO:14 or a fragment thereof.

351. The composition of any one of claims 331-347, wherein the CAR polypeptide comprises an amino acid sequence having at least about 80% sequence identity to SEQ ID NO:15 or a fragment thereof.

352. The composition of any one of claims 331-351, wherein the immune effector cell is transfected with an expression vector comprising an isolated nucleic acid sequences encoding the CAR polypeptide.

353. The composition of claim 352, wherein the vector is a lentiviral vector, a gamma-retroviral vector, an adenoviral vector, or an adeno-associated viral vector

354. The composition of any one of claims 331-353, wherein the immune effector cell is selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof.

355. The composition of any one of claims 331-354, wherein the immune effector cell is a Natural Killer (NK) cell.

356. The composition of any one of claims 331-355, wherein the composition comprises a pharmaceutically acceptable carrier.

357. The composition of claims 331-356, wherein the amount of cells ranges from about 10⁴ up to about 10⁸ cells per kg body weight of the mammal.

358. The composition of any one of claims 331-357, wherein the cancer is a solid tumor cancer.

359. The composition of claim 358, wherein the solid tumor cancer is sarcoma, and wherein the sarcoma comprises rhabdomyosarcoma, Ewing sarcoma, clear cell sarcoma, or leiomyosarcoma.

360. The composition of claim 359, wherein the sarcoma is rhabdomyosarcoma.

361. The composition of claim 359, wherein the sarcoma is Ewing sarcoma.

362. The composition of claim 358, wherein the solid tumor cancer is carcinoma, and wherein the carcinoma comprises melanoma or prostatic adenocarcinoma.

363. The composition of any one of claims 331-362, further comprising a second CAR polypeptide comprising an NKG2D receptor or fragment thereof.

364. The composition of claim 363, wherein expression of the first and second CAR polypeptides by an immune effector cell increases the likelihood of NK activation at tumor sites.

365. The composition according to claim 364, wherein the immune effector cell promotes an immune response against a cancer when the antibody of the CAR polypeptide binds MUC18 or a binding region thereof on a target cell and/or the extracellular domain of the NKG2D receptor of the second CAR polypeptide binds one or more NKG2D ligand binding targets on the target cell.

366. A method for stimulating an immune cell-mediated immune response to a target cell population and/or tissue in a mammal, wherein the target cell population and/or tissue express MUC18, the method comprising administering to a mammal in need thereof a therapeutically effective amount of a composition comprising immune effector cells expressing a chimeric antigen receptor (CAR) polypeptide comprising one or more antibodies or fragments thereof that binds to one or more cancer-associated antigens comprising MUC18, CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, NKG2D, NY-ESO-1, PSCA, WT-1, or binding regions thereof.

367. The method of claim 366, wherein the CAR polypeptide comprises:
the one or more antibodies or fragments thereof;
one or more extracellular spacers;
a transmembrane domain;
at least one costimulatory signaling region; and
optionally, a detection molecule.

368. The method of claim 366 or 367, wherein the one or more cancer-associated antigens or binding regions thereof bound by the one or more antibodies or fragments thereof is MUC18.

369. The method of any one of claims 366-368, wherein the one or more antibodies are scFv monoclonal antibodies.

370. The method of claim 366-369, wherein the one or more antibodies are anti-MUC18 scFv monoclonal antibodies.

371. The method of claim 370, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

372. The method according to claim 370 or 371, wherein the anti-MUC18 antibody binds to MUC18 or a binding region thereof expressed by sarcoma cells, carcinoma cells, or a combination thereof.

373. The method according to any one of claims 370-372, wherein the anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

374. The method according to any one of claims 370-372, wherein said anti-MUC18 antibody binds to MUC18 or a binding region thereof expressed by carcinoma cells comprising melanoma cells or prostatic adenocarcinoma cells.

375. The method according to any one of claims 367-374, wherein the CAR polypeptide does not comprise a CD3 zeta signaling domain.

376. The method of any one of claims 367-375, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

377. The method of any one of claims 367-376, wherein the at least one costimulatory signaling region comprises SEQ ID NO:8.

378. The method of any one of claims 367-376, wherein the at least one costimulatory signaling region comprises SEQ ID NO:9.

379. The method of any one of claims 367-376, wherein the at least one costimulatory signaling region comprises SEQ ID NO:10.

380. The method of any one of claims 367-376, wherein the at least one costimulatory signaling region comprises SEQ ID NO:11.

381. The method of any one of claims 367-380, wherein the one or more extracellular spacers comprise an IgG1 sequence, an IgG4, sequence, or a combination thereof.

382. The method of any one of claims 367-381, wherein the transmembrane domain comprises SEQ ID NO:7.

383. The method of any one of claims 366-382, wherein the CAR polypeptide comprises an amino acid sequence having at least about 80% sequence identity to SEQ ID NO: 12 or a fragment thereof.

384. The method of any one of claims 366-382, wherein the CAR polypeptide comprises an amino acid sequence having at least about 80% sequence identity to SEQ ID NO: 13 or a fragment thereof.

385. The method of any one of claims 366-382, wherein the CAR polypeptide comprises an amino acid sequence having at least about 80% sequence identity to SEQ ID NO: 14 or a fragment thereof.

386. The method of any one of claims 366-382, wherein the CAR polypeptide comprises an amino acid sequence having at least about 80% sequence identity to SEQ ID NO:15 or a fragment thereof.

387. The method of any one of claims 366-386, wherein the immune effector cells are transfected with an expression vector comprising an isolated nucleic acid sequence encoding the CAR polypeptide.

388. The method of claim 387, wherein the vector is a lentiviral vector, a gamma-retroviral vector, an adenoviral vector, or an adeno-associated viral vector.

389. The method of any one of claims 366-388, wherein the immune effector cells are selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof.

390. The method of any one of claims 366-389, wherein the immune effector cells are Natural Killer (NK) cells.

391. The method of any one of claims 366-390, wherein the composition comprises a pharmaceutically acceptable carrier.

392. The method of claim 366-391, wherein the amount of cells ranges from about 10⁴ up to about 10⁸ cells per kg body weight of the mammal.

393. The method of any one of claim 366-392, wherein the target cell population and/or tissue comprises solid tumor cancer cells and/or tissue.

394. The method of claim 393, wherein the solid tumor cancer cells and/or tissue comprises sarcoma cells and/or tissue, carcinoma cells and/or tissue, or a combination thereof.

395. The method of claim 394, wherein the sarcoma cells and/or tissue comprises rhabdomyosarcoma, Ewing sarcoma, clear cell sarcoma, or leiomyosarcoma cells and/or tissue.

396. The method of claim 394, wherein the carcinoma cells and/or tissue comprises melanoma or prostatic adenocarcinoma cells and/or tissue.

397. The method of any one of claims 366-396, wherein the composition further comprises a second CAR polypeptide comprising an NKG2D receptor or fragment thereof.

398. The method of claim 397, wherein expression of the first and second CAR polypeptides by immune effector cells increases the likelihood of NK activation at tumor sites.

399. The method according to claim 398, wherein the immune effector cells promote an immune response against a cancer when the antibody of the CAR polypeptide binds MUC18 or a binding region thereof on a target cell and/or the extracellular domain of the NKG2D receptor of the second CAR polypeptide binds one or more NKG2D ligand binding targets on the target cell.

400. A method of generating a persisting population of genetically engineered immune cells in a mammal, said method comprising administering to the mammal a therapeutically effective amount of a composition comprising immune effector cells genetically modified to express a chimeric antigen receptor (CAR) polypeptide comprising one or more antibodies or fragments thereof that binds to one or more cancer-associated antigens comprising MUC18, CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, NKG2D, NY-ESO-1, PSCA, WT-1, or binding regions thereof, wherein the persisting population of genetically engineered immune cells persists in the mammal for at least about one month up to at least about one year after administration.

401. The method of claim 400, wherein the CAR polypeptide comprises:
the one or more antibodies or fragments thereof;
one or more extracellular spacers;
a transmembrane domain;
at least one costimulatory signaling region; and
optionally, a detection molecule.

402. The method of claim 400 or 401, wherein the one or more cancer-associated antigens or binding regions thereof bound by the one or more antibodies or fragments thereof is MUC18.

403. The method of any one of claims 400-402, wherein the one or more antibodies are scFv monoclonal antibodies.

404. The method of any one of claims 400-403, wherein the one or more antibodies are anti-MUC18 scFv monoclonal antibodies.

405. The method of claim 404, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

406. The method according to claim 404 or 405, wherein the anti-MUC18 antibody binds to MUC18 or a binding region thereof expressed by sarcoma cells, carcinoma cells, or a combination thereof.

407. The method according to any one of claims 404-406, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

408. The method according to any one of claims 404-406, wherein said anti-MUC18 antibody binds to MUC18 or a binding region thereof expressed carcinoma cells comprising melanoma cells or prostatic adenocarcinoma cells.

409. The method according to any one of claims 401-408, wherein the CAR polypeptide does not comprise a CD3 zeta signaling domain.

410. The method of any one of claims 401-409, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

411. The method of any one of claims 401-410, wherein the at least one costimulatory signaling region comprises SEQ ID NO:8.

412. The method of any one of claims 401-410, wherein the at least one costimulatory signaling region comprises SEQ ID NO:9.

413. The method of any one of claims 401-410, wherein the at least one costimulatory signaling region comprises SEQ ID NO:10.

414. The method of any one of claims 401-410, wherein the at least one costimulatory signaling region comprises SEQ ID NO:11.

415. The method of any one of claims 401-414, wherein the one or more extracellular spacers comprise an IgG1 sequence, an IgG4, sequence, or a combination thereof.

416. The method of any one of claims 401-415, wherein the transmembrane domain comprises SEQ ID NO:7.

417. The method of any one of claims 400-416, wherein the CAR polypeptide comprises an amino acid sequence having at least about 80% sequence identity to SEQ ID NO:12 or a fragment thereof.

418. The method of any one of claims 400-416, wherein the CAR polypeptide comprises an amino acid sequence having at least about 80% sequence identity to SEQ ID NO:13 or a fragment thereof.

419. The method of any one of claims 400-416, wherein the CAR polypeptide comprises an amino acid sequence having at least about 80% sequence identity to SEQ ID NO:14 or a fragment thereof.

420. The method of any one of claims 400-416, wherein the CAR polypeptide comprises an amino acid sequence having at least about 80% sequence identity to SEQ ID NO:15 or a fragment thereof.

421. The method of any one of claims 400-420, wherein the immune effector cells are transfected with an expression vector comprising an isolated nucleic acid sequence encoding the CAR polypeptide.

422. The method of claim 421, wherein the vector is a lentiviral vector, a gamma-retroviral vector, a lentiviral vector, a gamma-retroviral vector, an adenoviral vector, an adeno-associated viral vector, or a combination thereof.

423. The method of any one of claims 400-422, wherein the immune effector cells are selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof.

424. The method of any one of claims 400-423, wherein the immune effector cells are Natural Killer (NK) cells.

425. The method of any one of claims 400-424, wherein the composition comprises a pharmaceutically acceptable carrier.

426. The method of claim 400-425, wherein the amount of cells ranges from about 10⁴ up to about 10⁸ cells per kg body weight of the mammal.

427. The method of any one of claims 400-426, wherein the cancer is a solid tumor cancer.

428. The method of claim 427, wherein the solid tumor cancer is sarcoma, and wherein the sarcoma comprises rhabdomyosarcoma, Ewing sarcoma, clear cell sarcoma, or leiomyosarcoma.

429. The method of claim 428, wherein the sarcoma is rhabdomyosarcoma.

430. The method of claim 428, wherein the sarcoma is Ewing sarcoma.

431. The method of claim 427, wherein the solid tumor cancer is carcinoma, and wherein the carcinoma comprises melanoma or prostatic adenocarcinoma.

432. The method of any one of claims 400-431, wherein the composition further comprises a second CAR polypeptide comprising an NKG2D receptor or fragment thereof.

433. The method of claim 432, wherein expression of the first and second CAR polypeptides by immune effector cells increases the likelihood of NK activation at tumor sites.

434. The method according to claim 433, wherein the immune effector cells promote an immune response against a cancer when the antibody of the CAR polypeptide binds MUC18 or a binding region thereof on a target cell and/or the extracellular domain of the NKG2D receptor of the second CAR polypeptide binds one or more NKG2D ligand binding targets on the target cell.

435. A method of expanding a population of genetically engineered immune cells in a mammal, the method comprising administering to the mammal a therapeutically effective amount of a composition comprising immune effector cells genetically modified to express a chimeric antigen receptor (CAR) polypeptide comprising one or more antibodies or fragments thereof that binds to one or more cancer-associated antigens comprising MUC18, CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, NKG2D, NY-ESO-1, PSCA, WT-1, or binding regions thereof, wherein the administered genetically engineered immune cell produces a population of progeny immune cells in the mammal.

436. The method of claim 435, wherein the CAR polypeptide comprises:
the one or more antibodies or fragments thereof;
one or more extracellular spacers;
a transmembrane domain;
at least one costimulatory signaling region; and
optionally, a detection molecule.

437. The method of claim 435 or 436, wherein the one or more cancer-associated antigens or binding regions thereof bound by the one or more antibodies or fragments thereof is MUC18.

438. The method of any one of claims 435-437, wherein the one or more antibodies are scFv monoclonal antibodies.

439. The method of claim 438, wherein the one or more antibodies are anti-MUC18 scFv monoclonal antibodies.

440. The method of claim 439, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

441. The method according to claim 439 or 440, wherein the anti-MUC18 antibody binds to MUC18 or a binding region thereof expressed by sarcoma cells, carcinoma cells, or a combination thereof.

442. The method according to any one of claims 439-441, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

443. The method according to any one of claims 439-441, wherein said anti-MUC18 antibody binds to MUC18 or a binding region thereof expressed by carcinoma cells comprising melanoma cells or prostatic adenocarcinoma cells.

444. The method according to any one of claims 436-443, wherein the CAR polypeptide does not comprise a CD3 zeta signaling domain.

445. The method of any one of claims 436-444, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

446. The method of any one of claims 436-445, wherein the at least one costimulatory signaling region comprises SEQ ID NO:8.

447. The method of any one of claims 436-445, wherein the at least one costimulatory signaling region comprises SEQ ID NO:9.

448. The method of any one of claims 436-445, wherein the at least one costimulatory signaling region comprises SEQ ID NO:10.

449. The method of any one of claims 436-445, wherein the at least one costimulatory signaling region comprises SEQ ID NO:11.

450. The method of any one of claims 436-449, wherein the one or more extracellular spacers comprise an IgG1 sequence, an IgG4, sequence, or a combination thereof.

451. The method of any one of claims 436-450, wherein the transmembrane domain comprises SEQ ID NO:7.

452. The method of any one of claims 435-451, wherein the CAR polypeptide comprises an amino acid sequence having at least about 80% sequence identity to SEQ ID NO:12 or a fragment thereof.

453. The method of any one of claims 435-451, wherein the CAR polypeptide comprises an amino acid sequence having at least about 80% sequence identity to SEQ ID NO:13 or a fragment thereof.

454. The method of any one of claims 435-451, wherein the CAR polypeptide comprises an amino acid sequence having at least about 80% sequence identity to SEQ ID NO:14 or a fragment thereof.

455. The method of any one of claims 435-451, wherein the CAR polypeptide comprises an amino acid sequence having at least about 80% sequence identity to SEQ ID NO:15 or a fragment thereof.

456. The method of any one of claims 435-455, wherein the immune effector cells are transfected with an expression vector comprising an isolated nucleic acid sequence encoding the CAR polypeptide.

457. The method of claim 456, wherein the vector is a lentiviral vector, a gamma-retroviral vector, a lentiviral vector, a gamma-retroviral vector, an adenoviral vector, an adeno-associated viral vector, or a combination thereof.

458. The method of any one of claims 435-457, wherein the immune effector cells are selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

459. The method of any one of claims 435-458, wherein the immune effector cell are Natural Killer (NK) cells.

460. The method of any one of claims 435-459, wherein the composition comprises a pharmaceutically acceptable carrier.

461. The method of claim 435-460, wherein the amount of cells ranges from about 10⁴ up to about 10⁸ cells per kg body weight of the mammal.

462. The method of any one of claims 435-461, wherein the cancer is a solid tumor cancer.

463. The method of claim 462, wherein the solid tumor cancer is sarcoma, and wherein the sarcoma comprises rhabdomyosarcoma, Ewing sarcoma, clear cell sarcoma, or leiomyosarcoma.

464. The method of claim 463, wherein the sarcoma is rhabdomyosarcoma.

465. The method of claim 463, wherein the sarcoma is Ewing sarcoma.

466. The method of claim 462, wherein the solid tumor cancer is carcinoma, and wherein the carcinoma comprises melanoma or prostatic adenocarcinoma.

467. The method of any one of claims 435-466, wherein the composition further comprises a second CAR polypeptide comprising an NKG2D receptor or fragment thereof.

468. The method of claim 467, wherein expression of the first and second CAR polypeptides by immune effector cells increases the likelihood of NK activation at tumor sites.

469. The method according to claim 468, wherein the immune effector cells promote an immune response against a cancer when the antibody of the CAR polypeptide binds MUC18 or a binding region thereof on a target cell and/or the extracellular domain of the NKG2D receptor of the second CAR polypeptide binds one or more NKG2D ligand binding targets on the target cell.

470. A method of treating a mammal with a cancer comprising cells that express MUC18 and/or one or more NKG2D ligands, the method comprising administering to a mammal in need thereof a therapeutically effective amount of a composition comprising immune effector cells genetically modified to express a chimeric antigen receptor (CAR) polypeptide comprising one or more antibodies or fragments thereof that binds to one or more cancer-associated antigens comprising MUC18, CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, NKG2D, NY-ESO-1, PSCA, WT-1, or binding regions thereof.

471. The method of claim 470, wherein the CAR polypeptide comprises:
the one or more antibodies or fragments thereof;
one or more extracellular spacers;
a transmembrane domain;
at least one costimulatory signaling region; and
optionally, a detection molecule.

472. The method of claim 470 or 471, wherein the one or more cancer-associated antigens or binding regions thereof bound by the one or more antibodies or fragments thereof is MUC18.

473. The method of any one of claims 470-472, wherein the one or more antibodies are scFv monoclonal antibodies.

474. The method of claim 473, wherein the one or more antibodies are anti-MUC18 scFv monoclonal antibodies.

475. The method of claim 474, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

476. The method according to claim 474 or 475, wherein the anti-MUC18 antibody binds to MUC18 or a binding region thereof expressed by sarcoma cells, carcinoma cells, or a combination thereof.

477. The method according to any one of claims 474-476, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

478. The method according to any one of claims 474-476, wherein said anti-MUC18 antibody binds to MUC18 or a binding region thereof expressed by carcinoma cells comprising melanoma cells or prostatic adenocarcinoma cells.

479. The method according to any one of claims 471-478, wherein the CAR polypeptide does not comprise a CD3 zeta signaling domain.

480. The method of any one of claims 471-479, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

481. The method of any one of claims 471-480, wherein the at least one costimulatory signaling region comprises SEQ ID NO:8.

482. The method of any one of claims 471-480, wherein the at least one costimulatory signaling region comprises SEQ ID NO:9.

483. The method of any one of claims 471-480, wherein the at least one costimulatory signaling region comprises SEQ ID NO:10.

484. The method of any one of claims 471-480, wherein the at least one costimulatory signaling region comprises SEQ ID NO:11.

485. The method of any one of claims 471-484, wherein the one or more extracellular spacers comprise an IgG1 sequence, an IgG4, sequence, or a combination thereof.

486. The method of any one of claims 471-485, wherein the transmembrane domain comprises SEQ ID NO:7.

487. The method of any one of claims 470-486, wherein the CAR polypeptide comprises an amino acid sequence having at least about 80% sequence identity to SEQ ID NO: 12 or a fragment thereof.

488. The method of any one of claims 470-486, wherein the CAR polypeptide comprises an amino acid sequence having at least about 80% sequence identity to SEQ ID NO:13 or a fragment thereof.

489. The method of any one of claims 470-486, wherein the CAR polypeptide comprises an amino acid sequence having at least about 80% sequence identity to SEQ ID NO: 14 or a fragment thereof.

490. The method of any one of claims 470-486, wherein the CAR polypeptide comprises an amino acid sequence having at least about 80% sequence identity to SEQ ID NO:15 or a fragment thereof.

491. The method of any one of claims 470-490, wherein the immune effector cells are transfected with an expression vector comprising an isolated nucleic acid sequence encoding the CAR polypeptide.

492. The method of claim 491, wherein the vector is a lentiviral vector, a gamma-retroviral vector, a lentiviral vector, a gamma-retroviral vector, an adenoviral vector, an adeno-associated viral vector, or a combination thereof.

493. The method of any one of claims 470-492, wherein the immune effector cells are selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and any combination thereof.

494. The method of any one of claims 470-493, wherein the immune effector cells are Natural Killer (NK) cells.

495. The method of any one of claims 470-494, wherein the composition comprises a pharmaceutically acceptable carrier.

496. The method of claim 470-495, wherein the amount of cells ranges from about 10⁴ up to about 10⁸ cells per kg body weight of the mammal.

497. The method of any one of claims 470-496, wherein the cancer is a solid tumor cancer.

498. The method of claim 497, wherein the solid tumor cancer is sarcoma, and wherein the sarcoma comprises rhabdomyosarcoma, Ewing sarcoma, clear cell sarcoma, or leiomyosarcoma.

499. The method of claim 498, wherein the sarcoma is rhabdomyosarcoma.

500. The method of claim 498, wherein the sarcoma is Ewing sarcoma.

501. The method of claim 497, wherein the solid tumor cancer is carcinoma, and wherein the carcinoma comprises melanoma or prostatic adenocarcinoma.

502. The method of any one of claims 470-501, wherein the composition further comprises a second CAR polypeptide comprising an NKG2D receptor or fragment thereof.

503. The method of claim 502, wherein expression of the first and second CAR polypeptides by immune effector cells increases the likelihood of NK activation at tumor sites.

504. The method according to claim 503, wherein the immune effector cells promote an immune response against a cancer when the antibody of the CAR polypeptide binds MUC18 or a binding region thereof on a target cell and/or the extracellular domain of the NKG2D receptor of the second CAR polypeptide binds one or more NKG2D ligand binding targets on the target cell.

505. A method for treatment of cancer comprising the steps of contacting an immune effector cell with a cancer cell or MDSC, M2-TAM, or Treg within the cancer microenvironment of a mammal and inducing apoptosis of the cancer cell, wherein the immune effector cell is genetically modified to express a chimeric antigen receptor (CAR) polypeptide comprising one or more antibodies or fragments thereof that binds to one or more cancer-associated antigens comprising MUC18, CD44v6, CAIX, CEA, CD133, c-Met, EGFR, EGFRvIII, Epcam, EphA2, GD2, GPC3, GUCY2C, HER1, HER2, ICAM-1, IL-13Rα2, IL-11Rα, Kras, Kras G12D, L1CAM, MAGE, MET, Mesothelin, MUC1, MUC16, NKG2D, NY-ESO-1, PSCA, WT-1, or binding regions thereof.

506. The method of claim 505, wherein the CAR polypeptide comprises:
the one or more antibodies or fragments thereof;
one or more extracellular spacers;
a transmembrane domain;
at least one costimulatory signaling region; and
optionally, a detection molecule.

507. The method of claim 505 or 506, wherein the one or more cancer-associated antigens or binding regions thereof bound by the one or more antibodies or fragments thereof is MUC18.

508. The method of any one of claims 505-507, wherein the one or more antibodies are scFv monoclonal antibodies.

509. The method of any one of claims 505-508, wherein the one or more antibodies are anti-MUC18 scFv monoclonal antibodies.

510. The method of claim 509, wherein the anti-MUC18 antibody comprises a variable heavy chain amino acid sequence having at least about 80% sequence identity to SEQ ID NO:2 or a fragment thereof and a variable light chain amino acid sequence having at least about 80% sequence identity to SEQ ID NO:4 or a fragment thereof, wherein the variable heavy chain and the variable light chain are linked by an amino acid sequence having at least about 80% sequence identity to SEQ ID NO:3 or a fragment thereof.

511. The method according to claim 509 or 510, wherein the anti-MUC18 antibody binds to MUC18 or a binding region thereof expressed by sarcoma cells, carcinoma cells, or a combination thereof.

512. The method according to any one of claim 509 or 511, wherein said anti-MUC18 antibody binds to MUC18 or a binding region thereof expressed by sarcoma cells comprising rhabdomyosarcoma cells, Ewing sarcoma cells, clear cell sarcoma cells, or leiomyosarcoma cells.

513. The method according to any one of claims 509-511, wherein said anti-MUC18 antibody binds to MUC18 or a binding region thereof expressed by carcinoma cells comprising melanoma cells or prostatic adenocarcinoma cells.

514. The method according to any one of claims 506-513, wherein the CAR polypeptide does not comprise a CD3 zeta signaling domain.

515. The method of any one of claims 506-514, wherein the at least one costimulatory signaling region comprises the intracellular domain of a costimulatory molecule selected from the group consisting of CD28, 2B4, DNAM-1, 4-1BB, OX40, CD30, CD40, ICOS, lymphocyte function-associated antigen-1 (LFA-1), CD2, CD7, LIGHT, NKG2C, and any combination thereof.

516. The method of any one of claims 506-515, wherein the at least one costimulatory signaling region comprises SEQ ID NO:8.

517. The method of any one of claims 506-515, wherein the at least one costimulatory signaling region comprises SEQ ID NO:9.

518. The method of any one of claims 506-515, wherein the at least one costimulatory signaling region comprises SEQ ID NO:10.

519. The method of any one of claims 506-515, wherein the at least one costimulatory signaling region comprises SEQ ID NO:11.

520. The method of any one of claims 506-519, wherein the one or more extracellular spacers comprise an IgG1 sequence, an IgG4, sequence, or a combination thereof.

521. The method of any one of claims 506-520, wherein the transmembrane domain comprises SEQ ID NO:7.

522. The method of any one of claims 505-521, wherein the CAR polypeptide comprises an amino acid sequence having at least about 80% sequence identity to SEQ ID NO:12 or a fragment thereof.

523. The method of any one of claims 505-521, wherein the CAR polypeptide comprises an amino acid sequence having at least about 80% sequence identity to SEQ ID NO:13 or a fragment thereof.

524. The method of any one of claims 505-521, wherein the CAR polypeptide comprises an amino acid sequence having at least about 80% sequence identity to SEQ ID NO: 14 or a fragment thereof.

525. The method of any one of claims 505-521, wherein the CAR polypeptide comprises an amino acid sequence having at least about 80% sequence identity to SEQ ID NO:15 or a fragment thereof.

526. The method of any one of claims 505-525, wherein the immune effector cell is transfected with an expression vector comprising an isolated nucleic acid sequence encoding the CAR polypeptide.

527. The method of claim 526, wherein the vector is a lentiviral vector, a gamma-retroviral vector, a lentiviral vector, a gamma-retroviral vector, an adenoviral vector, an adeno-associated viral vector, or a combination thereof.

528. The method of any one of claims 505-527, wherein the immune effector cell is selected from the group consisting of an αβ-T cell, a γδ-T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

529. The method of any one of claims 505-528, wherein the immune effector cell is a Natural Killer (NK) cell.

530. The method of any one of claims 505-529, wherein the composition comprises a pharmaceutically acceptable carrier.

531. The method of claim 530, wherein the amount of cells ranges from about 10⁴ up to about 10⁸ cells per kg body weight of the mammal.

532. The method of any one of claims 505-531, wherein the cancer is a solid tumor cancer.

533. The method of claim 532, wherein the solid tumor cancer is sarcoma, and wherein the sarcoma comprises rhabdomyosarcoma, Ewing sarcoma, clear cell sarcoma, or leiomyosarcoma.

534. The method of claim 533, wherein the sarcoma is rhabdomyosarcoma.

535. The method of claim 533, wherein the sarcoma is Ewing sarcoma.

536. The method of claim 532, wherein the solid tumor cancer is carcinoma, and wherein the carcinoma comprises melanoma or prostatic adenocarcinoma.

537. The method of any one of claims 505-536, wherein the composition further comprises a second CAR polypeptide comprising an NKG2D receptor or fragment thereof.

538. The method of claim 537, wherein expression of the first and second CAR polypeptides by an immune effector cell increases the likelihood of NK activation at tumor sites.

539. The method according to claim 538, wherein the immune effector cell promotes an immune response against a cancer when the antibody of the CAR polypeptide binds MUC18 or a binding region thereof on a target cell and/or the extracellular domain of the NKG2D receptor of the second CAR polypeptide binds one or more NKG2D ligand binding targets on the target cell.


SEQUENCE LISTING (82 sequences)

SEQ ID NO: 1 - protein, 21 aa, Artificial Sequence
  Description of Artificial Sequence Synthetic peptide
MALPVTALLLPLALLLHAARP

SEQ ID NO: 2 - protein, 121 aa, Artificial Sequence
  Description of Artificial Sequence Synthetic polypeptide
QVQLQESGPGLVKPSETLSLTCTVSGGSISSYYWSWIRQPPGKGLEWIGYIYYTWTSNYNPSLKSRVTISVDTSKNQFSLRLSSVTAADTAVYYCARDQGQWLLPDAFDIWGQGTMVTVSS

SEQ ID NO: 3 - protein, 15 aa, Artificial Sequence
  Description of Artificial Sequence Synthetic peptide
GGGGSGGGGSGGGGS

SEQ ID NO: 4 - protein, 112 aa, Artificial Sequence
  Description of Artificial Sequence Synthetic polypeptide
DIVMTQSPLSLPVTPGEPASISCRSSQSLLRSNGYNYLDWYLQKPGQSPHLLIYLGSNRASGVPDRFSGSGSGTDFTLKISRVEAEDVGVYYCMQAQQSPITFGQGTRLEIK

SEQ ID NO: 5 - protein, 12 aa, Artificial Sequence
  Description of Artificial Sequence Synthetic peptide
ESKYGPPCPPCP

SEQ ID NO: 6 - protein, 107 aa, Artificial Sequence
  Description of Artificial Sequence Synthetic polypeptide
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 29 aa, Artificial Sequence
  Description of Artificial Sequence Synthetic peptide
FWVLVVVGGVLACYSLLVTVAFIIFWVRS

SEQ ID NO: 8 - protein, 120 aa, Artificial Sequence
  Description of Artificial Sequence Synthetic polypeptide
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 9 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 10 - protein, 61 aa, Artificial Sequence
  Description of Artificial Sequence Synthetic polypeptide
NRRRRRERRDLFTESWDTQKAPNNYRSPISTSQPTNQSMDDTREDIYVNYPTFSRRPKTRV

SEQ ID NO: 11 - protein, 36 aa, Artificial Sequence
  Description of Artificial Sequence Synthetic polypeptide
RDQRLPPDAHKPPGGGSFRTPIQEEQADAHSTLAKI

SEQ ID NO: 12 - protein, 537 aa, Artificial Sequence
  Description of Artificial Sequence Synthetic polypeptide
MALPVTALLLPLALLLHAARPQVQLQESGPGLVKPSETLSLTCTVSGGSISSYYWSWIRQPPGKGLEWIGYIYYTWTSNYNPSLKSRVTISVDTSKNQFSLRLSSVTAADTAVYYCARDQGQWLLPDAFDIWGQGTMVTVSSGGGGSGGGGSGGGGSDIVMTQSPLSLPVTPGEPASISCRSSQSLLRSNGYNYLDWYLQKPGQSPHLLIYLGSNRASGVPDRFSGSGSGTDFTLKISRVEAEDVGVYYCMQAQQSPITFGQGTRLEIKESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 13 - protein, 459 aa, Artificial Sequence
  Description of Artificial Sequence Synthetic polypeptide
MALPVTALLLPLALLLHAARPQVQLQESGPGLVKPSETLSLTCTVSGGSISSYYWSWIRQPPGKGLEWIGYIYYTWTSNYNPSLKSRVTISVDTSKNQFSLRLSSVTAADTAVYYCARDQGQWLLPDAFDIWGQGTMVTVSSGGGGSGGGGSGGGGSDIVMTQSPLSLPVTPGEPASISCRSSQSLLRSNGYNYLDWYLQKPGQSPHLLIYLGSNRASGVPDRFSGSGSGTDFTLKISRVEAEDVGVYYCMQAQQSPITFGQGTRLEIKESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRGRKKLLYIFKQPFMRPVQTTQEEDGCSCRFPEEEEGGCEL

SEQ ID NO: 14 - protein, 478 aa, Artificial Sequence
  Description of Artificial Sequence Synthetic polypeptide
MALPVTALLLPLALLLHAARPQVQLQESGPGLVKPSETLSLTCTVSGGSISSYYWSWIRQPPGKGLEWIGYIYYTWTSNYNPSLKSRVTISVDTSKNQFSLRLSSVTAADTAVYYCARDQGQWLLPDAFDIWGQGTMVTVSSGGGGSGGGGSGGGGSDIVMTQSPLSLPVTPGEPASISCRSSQSLLRSNGYNYLDWYLQKPGQSPHLLIYLGSNRASGVPDRFSGSGSGTDFTLKISRVEAEDVGVYYCMQAQQSPITFGQGTRLEIKESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSNRRRRRERRDLFTESWDTQKAPNNYRSPISTSQPTNQSMDDTREDIYVNYPTFSRRPKTRV

SEQ ID NO: 15 - protein, 453 aa, Artificial Sequence
  Description of Artificial Sequence Synthetic polypeptide
MALPVTALLLPLALLLHAARPQVQLQESGPGLVKPSETLSLTCTVSGGSISSYYWSWIRQPPGKGLEWIGYIYYTWTSNYNPSLKSRVTISVDTSKNQFSLRLSSVTAADTAVYYCARDQGQWLLPDAFDIWGQGTMVTVSSGGGGSGGGGSGGGGSDIVMTQSPLSLPVTPGEPASISCRSSQSLLRSNGYNYLDWYLQKPGQSPHLLIYLGSNRASGVPDRFSGSGSGTDFTLKISRVEAEDVGVYYCMQAQQSPITFGQGTRLEIKESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSRDQRLPPDAHKPPGGGSFRTPIQEEQADAHSTLAKI

SEQ ID NO: 16 - protein, 113 aa, Artificial Sequence
  Description of Artificial Sequence Synthetic polypeptide
MRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 215 aa, Artificial Sequence
  Description of Artificial Sequence Synthetic polypeptide
GWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 18 - protein, 186 aa, Artificial Sequence
  Description of Artificial Sequence Synthetic polypeptide
YVNSAPLPPPPLTLLAEAAWNKAGVRLSICYFPPYCRLLAMGPGNLALSSRAFLGVFPLSPKECKVCMSRKQFLWKLLEDKQRLRPFAGSGTPHLATGASAAKSHVYKIHLQRRHNPSATLVGLWKESNGSPQAYSTRGRMPRRYPIVWDLIWGLGAHALHVFSRGKTSRPPEPRGRGFPLKNTII

SEQ ID NO: 19 - protein, 333 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 20 - protein, 847 aa, Artificial Sequence
  Description of Artificial Sequence Synthetic polypeptide
MRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVYVNSAPLPPPPLTLLAEAAWNKAGVRLSICYFPPYCRLLAMGPGNLALSSRAFLGVFPLSPKECKVCMSRKQFLWKLLEDKQRLRPFAGSGTPHLATGASAAKSHVYKIHLQRRHNPSATLVGLWKESNGSPQAYSTRGRMPRRYPIVWDLIWGLGAHALHVFSRGKTSRPPEPRGRGFPLKNTIIMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 21 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 22 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 23 - protein, 15 aa, Artificial Sequence
  Description of Artificial Sequence Synthetic peptide
EPKSCDKTHTCPPCP

SEQ ID NO: 24 - protein, 12 aa, Artificial Sequence
  Description of Artificial Sequence Synthetic peptide
ERKCCVECPPCP

SEQ ID NO: 25 - protein, 17 aa, Artificial Sequence
  Description of Artificial Sequence Synthetic peptide
ELKTPLGDTTHTCPRCP

SEQ ID NO: 26 - protein, 12 aa, Artificial Sequence
  Description of Artificial Sequence Synthetic peptide
ESKYGPPCPSCP

SEQ ID NO: 27 - protein, 23 aa, Artificial Sequence
  Description of Artificial Sequence Synthetic peptide
LGLLVAGVLVLLVSLGVAIHLCC

SEQ ID NO: 28 - protein, 25 aa, Artificial Sequence
  Description of Artificial Sequence Synthetic peptide
ALIVLGGVAGLLLFIGLGIFFCVRC

SEQ ID NO: 29 - protein, 23 aa, Artificial Sequence
  Description of Artificial Sequence Synthetic peptide
LCYLLDGILFIYGVILTALFLRV

SEQ ID NO: 30 - protein, 26 aa, Artificial Sequence
  Description of Artificial Sequence Synthetic peptide
WVLVVVGGVLACYSLLVTVAFIIFWV

SEQ ID NO: 31 - protein, 26 aa, Artificial Sequence
  Description of Artificial Sequence Synthetic peptide
VAAILGLGLVLGLLGPLAILLALYLL

SEQ ID NO: 32 - protein, 24 aa, Artificial Sequence
  Description of Artificial Sequence Synthetic peptide
ALPAALAVISFLLGLGLGVACVLA

SEQ ID NO: 33 - protein, 113 aa, Artificial Sequence
  Description of Artificial Sequence Synthetic polypeptide
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 34 - protein, 112 aa, Artificial Sequence
  Description of Artificial Sequence Synthetic polypeptide
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 35 - protein, 102 aa, Artificial Sequence
  Description of Artificial Sequence Synthetic polypeptide
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 36 - protein, 101 aa, Artificial Sequence
  Description of Artificial Sequence Synthetic polypeptide
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 37 - protein, 21 aa, Artificial Sequence
  Description of Artificial Sequence Synthetic peptide
ESPYQELQGQRSDVYSDLNTQ

SEQ ID NO: 38 - protein, 86 aa, Artificial Sequence
  Description of Artificial Sequence Synthetic polypeptide
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 39 - protein, 21 aa, Artificial Sequence
  Description of Artificial Sequence Synthetic peptide
DGVYTGLSTRNQETYETLKHE

SEQ ID NO: 40 - protein, 171 aa, Artificial Sequence
  Description of Artificial Sequence Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 41 - protein, 127 aa, Artificial Sequence
  Description of Artificial Sequence Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 42 - protein, 21 aa, Artificial Sequence
  Description of Artificial Sequence Synthetic peptide
DQVYQPLRDRDDAQYSHLGGN

SEQ ID NO: 43 - protein, 207 aa, Artificial Sequence
  Description of Artificial Sequence Synthetic polypeptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 44 - protein, 21 aa, Artificial Sequence
  Description of Artificial Sequence Synthetic peptide
NPDYEPIRKGQRDLYSGLNQR

SEQ ID NO: 45 - protein, 182 aa, Artificial Sequence
  Description of Artificial Sequence Synthetic polypeptide
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 46 - protein, 21 aa, Artificial Sequence
  Description of Artificial Sequence Synthetic peptide
DQLYQPLKDREDDQYSHLQGN

SEQ ID NO: 47 - protein, 163 aa, Artificial Sequence
  Description of Artificial Sequence Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 164 aa, Artificial Sequence
  Description of Artificial Sequence Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 49 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 112 aa, Artificial Sequence; identical to SEQ ID NO: 49
  Description of Artificial Sequence Synthetic polypeptide

SEQ ID NO: 51 - protein, 21 aa, Artificial Sequence
  Description of Artificial Sequence Synthetic peptide
NQLYNELNLGRREEYDVLDKR

SEQ ID NO: 52 - protein, 22 aa, Artificial Sequence
  Description of Artificial Sequence Synthetic peptide
EGLYNELQKDKMAEAYSEIGMK

SEQ ID NO: 53 - protein, 21 aa, Artificial Sequence
  Description of Artificial Sequence Synthetic peptide
DGLYQGLSTATKDTYDALHMQ

SEQ ID NO: 54 - protein, 226 aa, Artificial Sequence
  Description of Artificial Sequence Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 55 - protein, 188 aa, Artificial Sequence
  Description of Artificial Sequence Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 56 - protein, 21 aa, Artificial Sequence
  Description of Artificial Sequence Synthetic peptide
ENLYEGLNLDDCSMYEDISRG

SEQ ID NO: 57 - protein, 68 aa, Artificial Sequence
  Description of Artificial Sequence Synthetic polypeptide
FWVLVVVGGVLACYSLLVTVAFIIFWVRSKRSRLLHSDYMNMTPRRPGPTRKHYQPYAPPRDFAAYRS

SEQ ID NO: 58 - protein, 68 aa, Artificial Sequence; identical to SEQ ID NO: 57
  Description of Artificial Sequence Synthetic polypeptide

SEQ ID NO: 59 - protein, 619 aa, Artificial Sequence
  Description of Artificial Sequence Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 60 - protein, 370 aa, Artificial Sequence
  Description of Artificial Sequence Synthetic polypeptide
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 61 - protein, 255 aa, Artificial Sequence
  Description of Artificial Sequence Synthetic polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 62 - protein, 336 aa, Artificial Sequence
  Description of Artificial Sequence Synthetic polypeptide
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 63 - protein, 277 aa, Artificial Sequence
  Description of Artificial Sequence Synthetic polypeptide
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 64 - protein, 93 aa, Artificial Sequence
  Description of Artificial Sequence Synthetic polypeptide
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 65 - protein, 92 aa, Artificial Sequence
  Description of Artificial Sequence Synthetic polypeptide
MIHLGHILFLLLLPVAAAQTTPGERSSLPAFYPGTSGSCSGCGSLSLPLLAGLVAADAVASLLIVGAVFLCARPRRSPAQDGKVYINMPGRG

SEQ ID NO: 66 - protein, 220 aa, Artificial Sequence
  Description of Artificial Sequence Synthetic polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 67 - protein, 199 aa, Artificial Sequence
  Description of Artificial Sequence Synthetic polypeptide
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 68 - protein, 289 aa, Artificial Sequence
  Description of Artificial Sequence Synthetic polypeptide
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 69 - protein, 260 aa, Artificial Sequence
  Description of Artificial Sequence Synthetic polypeptide
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 70 - protein, 595 aa, Artificial Sequence
  Description of Artificial Sequence Synthetic polypeptide
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 71 - protein, 241 aa, Artificial Sequence
  Description of Artificial Sequence Synthetic polypeptide
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 72 - protein, 283 aa, Artificial Sequence
  Description of Artificial Sequence Synthetic polypeptide
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 73 - protein, 277 aa, Artificial Sequence
  Description of Artificial Sequence Synthetic polypeptide
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 74 - protein, 1170 aa, Artificial Sequence
  Description of Artificial Sequence Synthetic polypeptide
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 75 - protein, 351 aa, Artificial Sequence
  Description of Artificial Sequence Synthetic polypeptide
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 76 - protein, 240 aa, Artificial Sequence
  Description of Artificial Sequence Synthetic polypeptide
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 77 - protein, 240 aa, Artificial Sequence
  Description of Artificial Sequence Synthetic polypeptide
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 78 - protein, 231 aa, Artificial Sequence
  Description of Artificial Sequence Synthetic polypeptide
MSKQRGTFSEVSLAQDPKRQQRKPKGNKSSISGTEQEIFQVELNLQNPSLNHQGIDKIYDCQGLLPPPEKLTAEVLGIICIVLMATVLKTIVLIPFLEQNNSSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 79 - protein, 9 aa, Artificial Sequence
  Description of Artificial Sequence Synthetic peptide
YPYDVPDYA

SEQ ID NO: 80 - protein, 8 aa, Artificial Sequence
  Description of Artificial Sequence Synthetic peptide
DYKDDDDK

SEQ ID NO: 81 - protein, 10 aa, Artificial Sequence
  Description of Artificial Sequence Synthetic peptide
EQKLISEEDL

SEQ ID NO: 82 - protein, 277 aa, Artificial Sequence [sequence omitted]